(19) United States
(12) Patent Application Publication
(10) Pub. No.: US 2022/0296712 A1
(43) Pub. Date: Sep. 22, 2022

(54) METHODS FOR TREATMENT USING PHTHALOCYANINE DYE-TARGETING MOLECULE CONJUGATES

(71) Applicant: Rakuten Medical, Inc., San Diego, CA (US)

(72) Inventors: Miguel GARCIA-GUZMAN, San Diego, CA (US); Jerry FONG, San Mateo, CA (US); C. Daniel DE MAGALHAES FILHO, La Jolla, CA (US)

(73) Assignee: Rakuten Medical, Inc., San Diego, CA (US)

(21) Appl. No.: 17/638,147

(22) PCT Filed: Sep. 2, 2020
(86) PCT No.: PCT/US2020/049024

§ 371 Date: Feb. 24, 2022

Related U.S. Application Data
(60) Provisional application No. 62/895,325, filed on Sep. 3, 2019.

(51) Int. Cl. A61K 41/00; A61K 47/68; A61P 35/00; C07K 16/28; A61K 45/06

(52) U.S. Cl. CPC A61K 41/0057; A61K 47/6849; A61P 35/00; C07K 16/2818; A61K 45/06; A61K 2039/505

(57) ABSTRACT

Provided are compositions, combinations, and methods and uses for treating a subject having a tumor or lesion, including those not responsive or resistant to prior therapeutic treatments, such as prior immune checkpoint inhibitor treatments. In some aspects, the methods include administering to the subject a targeting molecule that binds CTLA-4 conjugated with phthalocyanine dye, such as IR700. In some cases, the methods include administering an immune modulatory agent. The tumor or lesion, in some cases, a first tumor, is illuminated with a wavelength of light suitable for the activation of the phthalocyanine dye of the conjugate. The provided methods and uses provide for growth inhibition, volume reduction, and elimination of tumors and tumor cells including primary tumors, metastatic tumor cells, and/or invasive tumor cells. Also provided are compositions, combinations, methods and uses for provoking or enhancing systemic and local immune responses and for synergistic responses against tumor growth.

### RELATED APPLICATIONS

This application claims priority from U.S. Provisional Application No. 62/895,325, filed Sep. 3, 2019, entitled “METHODS FOR TREATMENT USING PHTHALOCYANINE DYE-TARGETING MOLECULE CONJUGATES,” the contents of which are incorporated by reference in their entirety.

### FIELD

The present disclosure relates to compositions, combinations, and methods and uses for treating a subject having a tumor or lesion, including those not responsive or resistant to prior therapeutic treatments, such as prior immune checkpoint inhibitor treatments. In some aspects, the methods include administering to the subject a targeting molecule that binds CTLA-4 conjugated with phthalocyanine dye, such as IR700. In some cases, the methods include administering an immune modulatory agent. The tumor or lesion, in some cases, a first tumor, is illuminated with a wavelength of light suitable for the activation of the phthalocyanine dye of the conjugate. The methods and uses described herein provide for growth inhibition, volume reduction, and elimination of tumors and tumor cells including primary tumors, metastatic tumor cells, and/or invasive tumor cells. The disclosure also relates to compositions, combinations, methods and uses for provoking or enhancing systemic and local immune responses and for synergistic responses against tumor growth in a subject having a cancer, such as a cancer comprising a first tumor, metastatic tumor cells, and/or invasive tumor cells.

### BACKGROUND

Every year many therapeutics for treating cancer are developed, including immune checkpoint inhibitors, small molecule targeted therapies, and other anticancer therapeutics. However, some patients are not responsive to those therapeutics, and a majority of cancer patients will eventually develop non-responsiveness or resistance to therapeutics they receive during their treatment courses, leading to disease progression and cancer-related deaths. Novel compositions and methods are urgently needed to address these clinical challenges.

### SUMMARY

Provided herein are methods of treating a tumor or lesion. In some of any embodiments, the methods involve identifying a subject having a tumor or lesion that is non-responsive to a prior therapeutic treatment. In some of any embodiments, the methods involve administering to the subject a conjugate that includes a phthalocyanine dye linked to a targeting molecule, wherein the targeting molecule binds to cytotoxic T-lymphocyte-associated protein 4 (CTLA-4). In some of any embodiments, after administering the conjugate, the methods involve illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the methods can additionally involve administering a first immune modulatory therapy to the subject. In some of any embodiments, the growth and/or increase in volume of the tumor or lesion in the subject is inhibited or reduced.

Provided herein are methods of treating a tumor or lesion that involve: identifying a subject having a tumor or lesion that is non-responsive to a prior therapeutic treatment; administering to the subject a conjugate that includes a phthalocyanine dye linked to a targeting molecule, wherein the targeting molecule binds to CTLA-4; and after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length; wherein the growth and/or increase in volume of the tumor or lesion in the subject is inhibited or reduced.

Provided herein are methods of treating a tumor or lesion that involve: identifying a subject having a tumor or lesion that is non-responsive to a prior therapeutic treatment; administering to the subject a conjugate that includes a phthalocyanine dye linked to a targeting molecule, wherein the targeting molecule binds to CTLA-4; after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and administering a first immune modulatory therapy to the subject; wherein the growth and/or increase in volume of the tumor or lesion in the subject is inhibited or reduced.

In some of any embodiments, the prior therapeutic treatment includes treatment with an immune modulatory agent, an immune checkpoint inhibitor, an anti-cancer agent, a therapeutic agent that acts against suppressor cells, and any combination thereof. In some of any embodiments, prior therapeutic treatment includes treatment with a PD-1 inhibitor, a PD-L1 inhibitor, a CTLA-4 inhibitor, or any combination thereof.

In some of any embodiments, the prior therapeutic treatment includes treatment with an antibody or antigen-binding fragment of the antibody. In some of any embodiments, the antibody or antigen-binding fragment binds to PD-1, CTLA-4 or PD-L1.

In some of any embodiments, the first immune modulatory therapy is administered prior to administering the conjugate. In some of any embodiments, the first immune modulatory therapy is administered between about 1-3 weeks prior to administering the conjugate. In some of any embodiments, the first immune modulatory therapy is administered 1, 2, 3, 4, 5, or more than 5 times prior to administering the conjugate.

In some of any embodiments, the first immune modulatory therapy is administered concurrently with administering the conjugate.

In some of any embodiments, the first immune modulatory therapy is administered subsequent to administering the conjugate. In some of any embodiments, the first immune modulatory therapy is administered 1, 2, 3, 4, 5, or more than 5 times subsequent to administering the conjugate. In some of any embodiments, the first immune modulatory therapy is administered between about 1 day and 4 weeks after administering the conjugate.

In some of any embodiments, the first immune modulatory therapy is administered prior to administering the conjugate and administered at least one additional time subsequent to administering the conjugate. In some of any embodiments, the first immune modulatory therapy is administered 1, 2 or 3 times prior to administering the conjugate. In some of any embodiments, the first immune modulatory therapy is administered between about 1-3 weeks prior to administering the conjugate.

In some of any embodiments, the first immune modulatory therapy is an adjuvant for enhancing innate activation or an adjuvant for enhancing adaptive activation. In some of any embodiments, the first immune modulatory therapy is a T cell agonist.

Also provided herein are methods of treating tumor or lesion resistant to treatment with a prior immune checkpoint inhibitor. In some of any embodiments, the methods involve: identifying a tumor or lesion in a subject that is non-responsive to or resistant to treatment with a prior immune checkpoint inhibitor. In some of any embodiments, the methods involve: administering to the subject a conjugate that includes a phthalocyanine dye linked to a targeting molecule, wherein the targeting molecule binds to CTLA-4 In some of any embodiments, the methods involve: after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the methods involve additionally administering a first immune checkpoint inhibitor. In some of any embodiments, the tumor or lesion exhibits sensitivity to the first immune checkpoint inhibitor.

Also provided herein are methods of treating tumor or lesion resistant to treatment with a prior immune checkpoint inhibitor that involve: identifying a tumor or lesion in a subject that is non-responsive to or resistant to treatment with a prior immune checkpoint inhibitor; administering to the subject a conjugate that includes a phthalocyanine dye linked to a targeting molecule, wherein the targeting molecule binds to CTLA-4; and after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the tumor or lesion exhibits sensitivity to the first immune checkpoint inhibitor.

Also provided herein are methods of treating tumor or lesion resistant to treatment with a prior immune checkpoint inhibitor that involve: identifying a tumor or lesion in a subject that is non-responsive to or resistant to treatment with a prior immune checkpoint inhibitor; administering to the subject a conjugate that includes a phthalocyanine dye linked to a targeting molecule, wherein the targeting molecule binds to CTLA-4; after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and administering a first immune checkpoint inhibitor, wherein the tumor or lesion exhibits sensitivity to the first immune checkpoint inhibitor.

In some of any embodiments, the prior immune checkpoint inhibitor is selected from the group consisting of a PD-1 inhibitor, a PD-L1 inhibitor or a CTLA-4 inhibitor.

In some of any embodiments, the subject has a second tumor or lesion that is not illuminated, and wherein the second tumor or lesion exhibits sensitivity to administering the first immune checkpoint inhibitor. In some of any embodiments, the subject has metastatic tumor cells and wherein the metastatic tumor cells exhibit sensitivity to administering the first immune checkpoint inhibitor.

In some of any embodiments, sensitivity includes a reduction or inhibition of tumor growth, a reduction in tumor cell metastasis, an increase in tumor cell killing, an increase in systemic immune response, an increase in new T cell priming, an increase in diversity of CD8 T cells or any combinations thereof.

In some of any embodiments, the first immune checkpoint inhibitor is a PD-1 inhibitor, a PD-L1 inhibitor or a CTLA-4 inhibitor. In some of any embodiments, the first immune checkpoint inhibitor includes an antibody or antigen-binding fragment of an antibody.

Also provided herein are methods of provoking a systemic immune response. In some of any embodiments, the methods involve administering to a subject a conjugate that includes a phthalocyanine dye linked to a targeting molecule, wherein the targeting molecule binds to CTLA-4. In some of any embodiments, the methods involve, after administering the conjugate, illuminating at the site of a first tumor or first lesion at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the methods involve administering a first immune modulatory therapy. In some of any embodiments, following the steps of the methods, the subject exhibits at least one systemic response in a second tumor or second lesion distal to the illuminated site.

Also provided herein are methods of provoking a systemic immune response that involve administering to a subject a conjugate that includes a phthalocyanine dye linked to a targeting molecule, wherein the targeting molecule binds to CTLA-4; and after administering the conjugate, illuminating at the site of a first tumor or first lesion at a wavelength of at or about 600 nm to at or about 850 nm and at a dose of from at or about 25 J/cm² to at or about 400 J/cm² or from at or about 2 J/cm fiber length to at or about 500 J/cm fiber length wherein, following the steps of the method, the subject exhibits at least one systemic response in a second tumor or second lesion distal to the illuminated site.

Also provided herein are methods of provoking a systemic immune response that involve administering to a subject a conjugate that includes a phthalocyanine dye linked to a targeting molecule, wherein the targeting molecule binds to CTLA-4; after administering the conjugate, illuminating at the site of a first tumor or first lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and administering a first immune modulatory therapy, wherein following the steps of the method, the subject exhibits at least one systemic response in a second tumor or second lesion distal to the illuminated site.

In some of any embodiments, the systemic response includes a systemic immune responsive feature. In some of any embodiments, the systemic immune responsive feature is selected from the group consisting of an increase in CD8 T cell infiltration, an increase in CD8 T cell activation, an increase in dendritic cell infiltration, an increase in dendritic cell activation, an increase in new T cell priming, an increase in T cell diversity or any combination thereof. In some of any embodiments, the systemic immune responsive feature includes an increase in one or more of a proinflammatory molecule, a proinflammatory cytokine, an immune cell activation marker, or T cell diversity. In some of any embodiments, the systemic immune responsive feature is assessed from a blood sample obtained from the subject.

Also provided herein are methods of provoking a local immune response. In some of any embodiments, the methods involve administering to a subject a conjugate that includes a phthalocyanine dye linked to a targeting molecule, wherein the targeting molecule binds to CTLA-4. In some of any embodiments, the methods involve, after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the methods involve additionally administering a first immune modulatory therapy. In some of any embodiments, following the steps of the methods, the subject exhibits at least one local response, and wherein the response is synergistic as compared to treatment with only the first immune modulatory therapy or as compared to treatment with the conjugate administration and illuminating alone.

Provided herein are methods of provoking a local immune response that involves: administering to a subject a conjugate that includes a phthalocyanine dye linked to a targeting molecule, wherein the targeting molecule binds to CTLA-4; and after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length, wherein, following the steps of the methods, the subject exhibits at least one local response, and wherein the response is synergistic as compared to treatment with only the first immune modulatory therapy or as compared to treatment with the conjugate administration and illuminating alone.

Provided herein are methods of provoking a local immune response that involves: administering to a subject a conjugate that includes a phthalocyanine dye linked to a targeting molecule, wherein the targeting molecule binds to CTLA-4; after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and administering a first immune modulatory, wherein, following the steps of the methods, the subject exhibits at least one local response, and wherein the response is synergistic as compared to treatment with only the first immune modulatory therapy or as compared to treatment with the conjugate administration and illuminating alone.

In some of any embodiments, the local response includes a local immune response. In some of any embodiments, the local immune response is selected from the group consisting of intratumoral Treg depletion, an increase in intratumoral CD8 T cell infiltration, an increase in intratumoral CD8 T cell activation, a decrease in myeloid suppressive cells, a Type I interferon response and any combination thereof. In some of any embodiments, the local immune response includes an increase in the tumor or tumor microenvironment of an anti-immune cell type or an immune activation marker.

In some of any embodiments, the first immune modulatory therapy includes treatment with a PD-1 inhibitor or a PD-L1 inhibitor. In some of any embodiments, the first immune modulatory therapy includes treatment with an antibody or antigen-binding fragment of an antibody. In some of any embodiments, the first immune modulatory therapy is selected from the group consisting of an adjuvant for enhanced innate activation, an adjuvant for enhanced adaptive activation and a T cell agonist.

In some of any embodiments, the methods also involve treatment with a second conjugate that includes a cancer targeting molecule conjugated to a phthalocyanine dye, and wherein at least one illuminating step is performed subsequent to administering the second conjugate.

Also provided herein are methods of treating a tumor or lesion. In some of any embodiments, the methods involve identifying a cold tumor or lesion in a subject. In some of any embodiments, the methods involve administering to the subject a conjugate that includes a phthalocyanine dye linked to a targeting molecule, wherein the targeting molecule binds to CTLA-4. In some of any embodiments, the methods involve after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the growth and/or increase in volume of the cold tumor or lesion in the subject is inhibited or reduced.

Also provided herein are methods of treating a tumor or lesion that involve: identifying a cold tumor or lesion in a subject; administering to the subject a conjugate that includes a phthalocyanine dye linked to a targeting molecule, wherein the targeting molecule binds to CTLA-4; and after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the growth and/or increase in volume of the cold tumor or lesion in the subject is inhibited or reduced.

In some of any embodiments, the inhibition of tumor growth is enhanced as compared to treatment with a naked or unconjugated CTLA-4 antibody.

In some of any embodiments, the cold tumor or lesion is identified by a high mutational burden or a tumor immune score. In some of any embodiments, the cold tumor or lesion is identified by status of expression of a PD-1 or a PD-L1 marker. In some of any embodiments, the cold tumor or lesion is identified based on failure of the tumor or lesion to respond to a PD-1 inhibitor or an PD-L1 inhibitor. In some of any embodiments, the cold tumor or lesion is identified by a liquid biopsy or a tissue biopsy.

In some of any embodiments, Treg cells are rapidly depleted in the tumor or tumor microenvironment following the illuminating step. In some of any embodiments, necrosis of the tumor cells occurs following the illuminating step.

In some of any embodiments, the targeting molecule includes an anti-CTLA-4 antibody or antigen-binding fragment thereof. In some of any embodiments, the anti-CTLA-4 antibody is selected from the group consisting of ipilimumab (YERVOY), tremelimumab, AGEN1181, AGEN1884, ADU-1064, BCD-145, and BCD-217.

Provided herein are methods of treating a tumor or a lesion that is non-responsive to or resistant to a prior immune checkpoint inhibitor therapy. In some of any embodiments, the methods involve (a) identifying a tumor or a lesion in a subject that is non-responsive to or resistant to treatment with a prior immune checkpoint inhibitor; (b) administering to the subject a conjugate comprising a phthalocyanine dye linked to a targeting molecule that binds to CTLA-4; (c) after administering the conjugate, illuminating the tumor or the lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and (d) administering a first immune checkpoint inhibitor, wherein the tumor or the lesion exhibits sensitivity to the first immune checkpoint inhibitor.

In some of any embodiments, sensitivity to the first immune checkpoint inhibitor comprises a reduction in volume, dimensions or mass of the tumor or the lesion, a less than 20% increase in volume or dimensions of the tumor or the lesion, or a reduction in the number of tumor cells.

In some of any embodiments, sensitivity to the first immune checkpoint inhibitor comprises a reduction in tumor cell metastasis, an increase in tumor cell killing, an increase in systemic immune response, an increase in new T cell priming, an increase in diversity of CD8⁺ T cells or any combinations thereof.

In some of any embodiments, sensitivity to the first immune checkpoint inhibitor comprises an increase in systemic immune response, and the systemic immune response is measured by one or more of a cytotoxic T lymphocyte (CTL) activity assay, an intratumoral T cell exhaustion assay, an intratumoral effector T cell expansion assay, a T cell receptor diversity assay, an activated CD8⁺ T cell assay, a circulating regulatory T cell (Treg) assay, an intratumoral Treg assay, or a CD8⁺ Tcell:Treg assay.

In some of any embodiments, the tumor or the lesion that is non-responsive or resistant is identified by a high mutational burden or a tumor immune score. In some of any embodiments, the tumor or the lesion that is non-responsive or resistant is identified by status of expression of a PD-1 or a PD-L1 biomarker. In some of any embodiments, the tumor or the lesion that is non-responsive or resistant is identified by a liquid biopsy or a tissue biopsy.

In some of any embodiments, the treatment with the prior immune checkpoint inhibitor comprises treatment with a PD-1 inhibitor, a PD-L1 inhibitor or a CTLA-4 inhibitor.

In some of any embodiments, the treatment with the prior immune checkpoint inhibitor comprises treatment with an anti-PD-1 antibody or antigen-binding fragment thereof. In some of any embodiments, the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

Provided herein are methods of provoking a systemic immune response. In some of any embodiments, the methods involve (a) administering to a subject a conjugate comprising a phthalocyanine dye linked to a targeting molecule that binds to CTLA-4; (b) after administering the conjugate, illuminating at the site of a first tumor or a first lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and (c) administering a first immune checkpoint inhibitor, wherein following steps (a), (b), and (c), the subject exhibits at least one systemic immune responsive feature in a location distal to the illuminated site.

In some of any embodiments, the at least one systemic immune responsive feature is selected from the group consisting of an increase in CD8⁺ T cell infiltration, an increase in CD8⁺ T cell activation, an increase in the CD8⁺:Treg ratio, an increase in natural killer cell infiltration, an increase in natural killer cell activation, an increase in dendritic cell infiltration, an increase in dendritic cell activation, an increase in new T cell priming, an increase in T cell diversity, and any combination thereof.

In some of any embodiments, the at least one systemic immune responsive feature comprises an increase in one or more of a proinflammatory molecule, a proinflammatory cytokine, or an immune cell activation marker.

In some of any embodiments, the at least one systemic immune responsive feature is assessed from a blood sample obtained from the subject.

In some of any embodiments, the location distal to the illuminated site is a second tumor or a second lesion that is not illuminated.

Provided herein are methods of provoking a local immune response comprising: (a) administering to a subject a conjugate comprising a phthalocyanine dye linked to a targeting molecule that binds to CTLA-4; (b) after administering the conjugate, illuminating the tumor or the lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and (c) administering a first immune checkpoint inhibitor, wherein following steps (a), (b), and (c), the subject exhibits at least one local immune responsive feature, and wherein the at least one local immune responsive feature is synergistic as compared to administering only the first immune checkpoint inhibitor or as compared to treatment only with the conjugate and the illuminating step.

In some of any embodiments, the at least one local immune responsive feature is selected from the group consisting of intratumoral Treg depletion, an increase in intratumoral CD8 T cell infiltration, an increase in intratumoral CD8 T cell activation, an increase in the intratumoral CD8⁺:Treg ratio, an increase in intratumoral natural killer cell infiltration, an increase in intratumoral natural killer cell activation, a decrease in myeloid suppressive cells, a Type I interferon response, and any combination thereof. In some of any embodiments, the at least one local immune responsive feature comprises an increase in an anti-immune cell type or an immune activation marker in the tumor or tumor microenvironment.

In some of any embodiments, the targeting molecule comprises an anti-CTLA-4 antibody or an antigen binding fragment thereof. In some of any embodiments, the anti-CTLA-4 antibody is selected from the group consisting of ipilimumab (YERVOY), tremelimumab, AGEN1181, AGEN1884, ADU-1064, BCD-145, CBT-509, and BCD-217.

In some of any embodiments, the first immune checkpoint inhibitor comprises an anti-PD-1 antibody or antigen-binding fragment thereof. In some of any embodiments, the first immune checkpoint inhibitor is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011), and antigen-binding fragments thereof.

In some of any embodiments, the first immune checkpoint inhibitor is administered concurrently with the administering the conjugate. In some of any embodiments, the first immune checkpoint inhibitor is administered within 24 hours of administering the conjugate.

In some of any embodiments, the first immune checkpoint inhibitor is administered prior to administering the conjugate. In some of any embodiments, the first immune checkpoint inhibitor is administered between about 1-3 weeks prior to administering the conjugate. In some of any embodiments, the first immune checkpoint inhibitor is administered 1, 2, 3, 4, 5 times, or more than 5 times prior to administering the conjugate.

In some of any embodiments, the method also involves administering the first immune checkpoint inhibitor subsequent to administering the conjugate. In some of any embodiments, the first immune checkpoint inhibitor is administered 1, 2, 3, 4, 5 times, or more than 5 times subsequent to administering the conjugate.

In some of any embodiments, the first immune checkpoint inhibitor is administered between about 1 day and about 4 weeks after administering the conjugate.

In some of any embodiments, the subject exhibits progressive disease or a stable disease following treatment with a prior immune checkpoint inhibitor.

In some of any embodiments, the tumor or the lesion that is non-responsive to or resistant to a prior immune checkpoint inhibitor therapy comprises a tumor or a lesion that exhibits a lack of reduction in volume, dimensions or mass of the tumor or the lesion, more than 20% increase in volume or dimensions of the tumor or the lesion, or an increase in the number of tumor cells, or a metastases.

In some of any embodiments, the subject comprises a second tumor or lesion that is not illuminated, and wherein the second tumor or lesion exhibits sensitivity to administering the first immune checkpoint inhibitor. In some of any embodiments, the subject comprises metastatic tumor cells and wherein the metastatic tumor cells exhibit sensitivity to administering the first immune checkpoint inhibitor.

In some of any embodiments, the subject does not experience a substantial reduction in systemic Treg cells.

In some of any embodiments, the subject exhibits a response at a site distal to the illuminated tumor or lesion, wherein the response is selected from the group consisting of an increase in CD8⁺ T cell infiltration, an increase in CD8⁺ T cell activation, an increase in the intratumoral CD8⁺:Treg ratio, an increase in intratumoral natural killer cell infiltration, an increase in intratumoral natural killer cell activation, an increase in dendritic cell infiltration, an increase in dendritic cell activation, an increase in new T cell priming, an increase in T cell diversity, increase in one or more of a proinflammatory molecule, a proinflammatory cytokine, an immune cell activation marker, and any combination thereof.

In some of any embodiments, the method results in a substantial decrease in the number, the frequency, the activity and/or the function of an intratumoral suppressor cell. In some of any embodiments, the intratumoral suppressor cell is selected from the group consisting of regulatory T cells, type II natural killer T cells, M2 macrophages, tumor associated fibroblast, myeloid-derived suppressor cell, and any combination thereof. In some of any embodiments, the method results in a substantial increase in the number or the frequency of intratumoral cytotoxic T effector cells, natural killer (NK) cells, other immune effector cells, or any combination thereof. In some of any embodiments, the method results in in a substantial increase in the activity or the function of intratumoral cytotoxic T effector cells, natural killer (NK) cells, other immune effector cells, or any combination thereof.

In some of any embodiments, necrosis of the tumor or the lesion occurs following the illuminating step.

In some of any embodiments, the phthalocyanine dye is a Si-phthalocyanine dye. In some of any embodiments, the Si-phthalocyanine dye is IR700.

In some of any embodiments, the first immune modulatory therapy or the first immune checkpoint inhibitor includes treatment with an anti-PD-1 antibody selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

In some of any embodiments, wherein the first immune modulatory therapy or the first immune checkpoint inhibitor includes treatment with an anti-PD-L1 antibody selected from the group consisting of atezolizumab (MPDL3280A, TECENTRIQ, RG7446), avelumab (BAVENCIO, MSB0010718C; M7824), durvalumab (MEDI4736, IMFINZI), LDP, NM-01, STI-3031 (IMC-001; STI-A1015), KN035, LY3300054, M7824 (MSB0011359C), BMS-936559, MSB2311, BCD-135, BGB-A333, CBT-502 (TQB-2450), cosibelimab (CK-301), CS1001 (WPB3155), FAZ053, MDX-1105, SHR-1316 (HTI-1088), TG-1501, ZKAB001 (STI-A1014), INBRX-105, MCLA-145, KN046, LY3415244, REGN3504, and HLX20.

In some of any embodiments, the illuminating step is carried out between 30 minutes and 96 hours after administering the conjugate. In some of any embodiments, the illuminating step is carried out 24 hours±4 hours after administering the conjugate. In some of any embodiments, the illuminating step is carried out at a wavelength of 690±40 nm. In some of any embodiments, the illuminating step is carried out at a dose of or about of 50 J/cm² or 100 J/cm of fiber length.

In some of any embodiments, the administration of the conjugate is repeated one or more times. In some of any such embodiments, after each repeated administration of the conjugate, the illuminating step is repeated.

In some of any embodiments, the methods also involve administering an additional therapeutic agent or anti-cancer treatment.

In some of any embodiments, the tumor or lesion is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

In some of any embodiments, the conjugate provides an effect independent of the number or activity of systemic regulatory T cells.

In some of any embodiments, the method results in a substantial increase in the number or frequency of intratumoral cytotoxic T effector cells, natural killer (NK) cells, other immune effector cells, or any combination thereof. In some of any embodiments, the method results in in a substantial increase in the activity or function of intratumoral cytotoxic T effector cells, natural killer (NK) cells, other immune effector cells, or any combination thereof. In some of any embodiments, the method results in a substantial decrease in the number or frequency and/or activity or function of an intratumoral suppressor cell. In some of any embodiments, the intratumoral suppressor cell is selected from the group consisting of regulatory T cells, type II natural killer T cells, M2 macrophages, tumor associated fibroblast, myeloid-derived suppressor cell, or any combination thereof.

### BRIEF DESCRIPTION OF THE DRAWINGS

FIG. 1 shows that anti-CTLA-4 antibody (Anti-CTLA4) or anti-CTLA-4 IR700 PIT (CTLA4-IR700 PIT) inhibited the growth of tumors with reduced immunoresponsiveness.

FIG. 2A shows that anti-CTLA-4-IR700 PIT (CTLA4-IR700 PIT) substantially inhibits growth of illuminated (left panel) and non-illuminated distal (right panel) CT26 murine colon carcinoma-derived tumors in vivo, and the inhibitory effect is greater than that observed for anti-CTLA-4-IR700 conjugate (CTLA-4-IR700) alone.

FIG. 2B shows that anti-CTLA-4-IR700 PIT (CTLA4-IR700 PIT) substantially inhibited growth of illuminated (left panel) and non-illuminated distal (right panel) MCA205 murine fibrosarcoma-derived tumors in vivo, and the inhibitory effect was greater than that observed for anti-CTLA-4-IR700 conjugate (CTLA-4-IR700) alone.

FIG. 3 shows the resistance of cold tumors to anti-CTLA-4 and anti-PD-1 therapies. Tumors derived from 4T1 murine mammary carcinoma cells showed diminished immunoresponsiveness, and were thus designated as “cold tumors.” The cold tumors were resistant to treatments of anti-CTLA-4 IR700 conjugate alone (CTLA4-IR700), or in combination with anti-PD-1 immune checkpoint inhibitor (CTLA4-IR700+anti-PD1).

FIG. 4A shows the resistance of “cold” tumors, exhibiting diminished immunoresponsiveness, to anti-CTLA-4 IR700 conjugate alone and anti-CTLA-4 PIT. In comparison to the control group (saline), neither anti-CTLA-4-IR700 conjugate (CTLA4-IR700) alone (without illumination) nor anti-CTLA-4 PIT (CTLA4-IR700 PIT) reduced the growth of tumors (FIG. 4A).

FIG. 4B shows that anti-CTLA-IR700 PIT (CTLA4-IR700 PIT; solid line) improved the survival of “cold” 4T1 tumor-bearing mice compared to saline (dotted line) or anti-CTLA-4-IR700 conjugate without illumination (CTLA4-IR700; dashed line).

FIG. 5 shows that anti-CTLA-4 PIT (CTLA-4-PIT) sensitized 4T1-derived “cold” tumors to anti-PD-1 antibody treatment.

FIG. 6 shows that anti-CTLA-4-IR700 PIT (CTLA-4 PIT) sensitized unilluminated, distal cold tumors to treatment with anti-PD-1 antibody. Anti-CTLA-4 PIT alone (CTLA-4 PIT) did not show a substantial inhibitory effect on the growth of cold tumors, but it sensitized the tumors to anti-PD-1 treatment (CTLA-4 PIT+anti-PD1)

FIG. 7 shows that the abscopal effect of anti-CTLA-4 PIT in combination with anti-PD-1 (Anti-CTLA-4) on unilluminated distal cold tumors did not require reduction of systemic regulatory T cells (*: p<0.001).

FIGS. 8A-8B show the depletion of intratumoral regulatory T cells (Tregs) in vivo in response to anti-CTLA-4-PIT (CTLA-4 PIT), compared to administration of saline or anti-CTLA4-IR700 conjugate alone (CTLA-4-IR700), at 2 hours (FIG. 8A) and at 7 days post-treatment (FIG. 8B). Administration of the anti-CTLA4-IR700 conjugate alone (CTLA-4-IR700) did not significantly decrease the percentage of intratumoral Tregs, compared to saline controls, at 2 hours (FIG. 8A), but a depletion of intratumoral Tregs was observed at 7 days post-treatment (FIG. 8B).

FIGS. 9A-9B show the increase in the intratumoral CD8⁺ T cell: regulatory T cell (CD8⁺:Treg) ratio in vivo in response to anti-CTLA-4-PIT (CTLA-4 PIT) at 2 hours (FIG. 9A) and 7 days post-treatment (FIG. 9B), compared to saline treatment. Administration of the anti-CTLA4-IR700 conjugate alone (CTLA-4-IR700) did not increase the CD8⁺:Treg ratio at 2 hours (FIG. 9A), but an increase in the CD8⁺:Treg ratio was observed at 7 days post-treatment (FIG. 9B).

FIG. 10 shows the increase in intratumoral activated CD8⁺ T cells (CD3⁺ CD8⁺ CD25⁺) in vivo 2 hours after anti-CTLA-4-PIT (CTLA-4-PIT) compared to administration of saline or anti-CTLA-4-IR700 conjugate alone (CTLA-4-IR700).

FIGS. 11A-11B show the sustained increase in intratumoral CD8⁺ T cell activation (percent CD3⁺ CD8⁺ Ki-67⁺ of CD45⁺ cells; FIG. 11A and percent CD3⁺ CD8⁺ CD69⁺ of CD45⁺ cells; FIG. 11B) in vivo 7 days after anti-CTLA-4-IR700 PIT (CTLA-4 PIT) compared to administration of saline or anti-CTLA-4-IR700 conjugate (CTLA-4-IR700) alone.

FIGS. 12A-12B show the increase in intratumoral activated natural killer (NK) cells (percent CD49b⁺ CD3⁻ Ki-67⁺ of CD45⁺ cells; FIG. 12A and percent CD49b⁺ CD3⁻CD69⁺ of CD45⁺ cells; FIG. 12B) in vivo 7 days after anti-CTLA-4-IR700 PIT (CTLA-4 PIT) compared to administration of saline or anti-CTLA-4-IR700 conjugate (CTLA-4-IR700) alone.

FIG. 13 shows the cytotoxicity against CT26 tumor cells or unrelated tumor cells after incubation with splenocytes obtained from complete response (CR) mice that were treated with an anti-CTLA-4-IR700 and illumination (CTLA-4 PIT) or anti-CTLA-4-IR700 conjugate (CTLA-4-IR700) alone, at effector: target ratios of 100:1, 33:1, 11:1, 3.7:1, 1.23:1 and 0.41:1 (or 100:1 for unrelated tumor cells), after being primed with tumor specific antigen.

FIGS. 14A-14B show the anti-tumor systemic immunity established in mice following anti-CTLA-4-IR700 PIT. The results showed tumor growth in naïve animals (FIG. 14A) and in complete responder (CR) mice re-challenged with tumor cells after previously established tumors were treated with anti-CTLA-4-IR700 PIT (FIG. 14B).

### DETAILED DESCRIPTION

Provided herein are compositions, combinations, and methods for treating a subject having a tumor or lesion, such as cold tumors and/or tumors or lesions that are not responsive to or resistant to prior therapeutic treatments, such as prior immune checkpoint inhibitor treatments and other prior anti-cancer therapeutic treatments. In some aspect, the provided embodiments involve administering to the subject a targeting molecule that binds cytotoxic T-lymphocyte-associated protein 4 (CTLA-4) conjugated with phthalocyanine dye, such as IR700. In some aspects, the provided embodiments involve illumination of the site of the tumor or the lesion. In some aspects, the illumination results in death of cells expressing CTLA-4 on the surface. In some aspects, the methods also involve the administration of an immune modulating agent, such as an immune checkpoint inhibitor, in combination with the phthalocyanine dye-targeting molecule conjugate.

In some aspects, the phthalocyanine dye-targeting molecule conjugate (e.g., anti-CTLA-4 antibody or antigen-binding fragment thereof conjugated to IR700), and, in some cases, an immune modulating agent, such as an immune checkpoint inhibitor, are employed in the provided methods and uses, such as in the provided methods and uses for treatment of a cancer. Uses include therapeutic uses of the compositions and combinations, for example, in methods, treatments, or treatment regimens. Uses include uses of the compositions and combinations in such methods and treatments, and uses of such compositions and combinations, in the preparation of a medicament in order to carry out such therapeutic methods. In some embodiments, the methods and uses thereby treat the cancer, such as cancers that include primary tumors, metastatic tumor cells and/or invasive tumor cells, such as an invasive cancer, an infiltrating cancer, or a metastatic cancer, in a subject. Also provided are compositions, combinations and methods for generating an enhanced response, for example, an enhanced response to a treatment or a therapy in a subject, e.g., a subject having a cancer or a tumor, such as an invasive cancer, an infiltrating cancer, or a metastatic cancer. In some aspects, also provided are methods and uses of such compositions and combinations in enhancing, provoking, augmenting, boosting or supporting the immune function, such as local and systemic immunity, in the subject. In some aspects, provided are methods of provoking a systemic immune response. In some aspects, provided are methods of provoking a local immune response.

The provided compositions, combinations, methods and uses can be used to treat cancers that include one or more first tumors or first lesions and/or one or more second tumors or second lesions, such as primary tumors, metastatic tumor cells and/or invasive tumor cells. In some embodiments, a phthalocyanine dye conjugated with a targeting molecule that binds CTLA-4 is used alone or in combination with an immune checkpoint inhibitor. The methods and uses described herein provide various advantages in treating cancers, e.g., metastatic cancers and/or invasive cancers, including without the need to locate and/or directly illuminate the metastatic tumor cells and/or invasive tumor cells. The disclosure also provides unexpected features in enhancing the systemic immunity in a subject, for example, against cancer recurrence.

The provided embodiments, in some contexts, are based on the observation that treatment of a cancer with a phthalocyanine dye-targeting molecule conjugate, such as an anti-CTLA-4 antibody-IR700 conjugate, followed by illumination of a first (e.g., a primary) tumor, results in not only treatment of the first tumor, but also results in effective treatment of a tumor that is distal to the illumination site (e.g., metastasized tumor), and effective treatment of a tumor that is introduced after the subject has a complete response following the treatment of the initial tumor, indicating a tumor-specific immune memory response. Surprisingly this response is not depended on the depletion of systemic regulatory T cells (Tregs) as observed with other therapies. The provided embodiments are based on a further observation that a combination treatment with an anti-CTLA4 antibody-IR700 conjugate and an immune checkpoint inhibitor, such as an anti-PD-1 antibody, results in striking synergistic effects in the treatment of both the illuminated first tumor and a distal tumor or a later-introduced tumor, such as a tumor comprising a secondary population of related tumor cells, a metastatic tumor and/or an invasive tumor. Accordingly, the provided compositions, combinations, methods and uses are demonstrated to provide a substantially improved and effective treatment of a cancer, including cancers that include a primary tumor or multiple primary tumors as well as metastatic tumor cells, for example metastatic cancers; and/or cancers that include a primary tumor or multiple primary tumors as well as invasive tumor cells, for example, invasive cancers without depleting systemic Tregs. The provided compositions, combinations, methods and uses can result in enhancement or improvement of the subject's immune response, e.g. systemic immune response against a cancer including immune memory response, that can be effective against tumors that may develop after the treatment.

One of the great challenges in treating cancer patients is the lack of responsiveness of cancers to therapeutics. Compositions and methods for treating such cancers are urgently needed. The provided embodiments, in some contexts, are based on the observation that, for tumors that are classified as “cold” tumors and for tumors and tumor cells that are not responsive to prior therapeutic treatments, for example, an immune checkpoint inhibitor, an anticancer agent, or a molecule against immune suppressor cells, treatment with a phthalocyanine dye-targeting molecule conjugate, such as an anti-CTLA-4 conjugate, followed by illumination at a tumor site (also referred to as “photoimmunotherapy” and “PIT”), results in a substantial inhibition of tumor growth. Furthermore, in combination with an immune modulatory therapy, such as an immune checkpoint inhibitor, a greater inhibitory effect on the growth of tumors can be observed than with either agent alone.

As used herein an “anti-CTLA-4 conjugate” can refer to a conjugate that has a CTLA-4 targeting molecule linked to a phthalocyanine dye. The CTLA-4 targeting molecule can include a CTLA-4 binding molecule, such as an anti-CTLA-4 antibody or antibody fragment (e.g., antigen binding fragment), or other protein, peptide or small molecule that binds to CTLA-4. An anti-CTLA-4 conjugate can include a Si-phthalocyanine dye, such as an IR700 dye.

As used herein, treatment with or administration of an anti-CTLA-4 conjugate is generally followed by illumination with a suitable wavelength of light, and it should be assumed that such illumination is part of the treatments and administrations of an anti-CTLA-4 conjugate unless specifically stated that an illumination step is not performed with the method.

The compositions, combinations, and methods herein can be used to treat cancers that have a low responsiveness or are substantially non-responsive to a prior therapeutic treatment, such as an immune modulatory agent, an immune checkpoint inhibitor, an anti-cancer agent, or a therapeutic agent that acts against immune suppressor cells. Also provided are compositions, combinations, and methods for treating a cancer that are resistant to a prior treatment, such as resistant to treatment with an immune checkpoint inhibitor. Further provided are compositions, combinations, and methods for provoking an immune response, including a local immune response and/or a systemic immune response in the treated subject. The compositions, combinations, and methods herein can be used to treat “cold” tumors and lesions.

The methods described herein provide various advantages in treating cancers, such as effective treatment of cancers that are not responsive to prior therapeutic treatments One of the advantages includes the treatment of metastatic cancers and/or invasive cancers without the need to locate and/or directly illuminate the metastatic tumor cells and/or invasive tumor cells. The provided methods and compositions also can provoke local and systemic immunity in a subject, for example, against tumor cells and cells in the tumor microenvironment.

Provided herein are compositions, combinations, and methods for treating a tumor or cancerous lesion, such as cancers that include a primary tumor or multiple primary tumors as well as metastatic tumor cells. In some cases, a treated subject may have one or more first tumors (e.g., primary tumors), metastatic tumor cells and invasive tumor cells.

In some instances, a tumor can be a “cold tumor” that has an immunosuppressive phenotype. Such cold tumors can have features including, but not limited to, a substantial reduction in numbers and/or activities or absence of intratumoral CD8⁺ T effector cells and/or substantial increase in numbers and/or activities of intratumoral immune suppressor cells. In some cases, a cold tumor or lesion has a high tumor mutational burden (TMB), an immune score indicative of low immunoresponsiveness, a Programmed cell death protein 1 (PD-1) or Programmed death-ligand 1 (PD-L1) marker status indicative of low immunoresponsiveness. In some instances, a cold tumor or lesion does not respond to a PD-1 or a PD-L1 inhibitor monotherapy.

In some embodiments, cold tumors or lesions can be treated with anti-CTLA-4 conjugate. In some embodiments, a combination treatment with an anti-CTLA-4 conjugate and an immune checkpoint inhibitor, such as an anti-PD-1 antibody, results in striking inhibitory effects on the growth of both the illuminated first (e.g., primary) tumor and a non-illuminated distal tumor.

Furthermore, for tumors that are resistant to or non-responsive to a treatment with an immune modulatory therapy, such as treatment with an immune checkpoint inhibitor (e.g., an anti-PD-1 antibody), a combination treatment with an anti-CTLA-4 conjugate and an immune checkpoint inhibitor, such as an anti-PD-1 antibody, results in unexpected inhibitory effects on the growth of both illuminated first tumor and a distal tumor, indicating a sensitization effect of the anti-CTLA-4 conjugate treatment on immune checkpoint inhibitors in treating cancers and tumor cells.

The provided compositions, combinations, and methods provide an effective treatment of a cancer, including first tumors or lesions and/or second tumors or lesions, such as primary tumors and metastatic cancer. The methods provided herein include treating a subject not responsive to or resistant to prior therapeutic treatments for a tumor or cancer, with an anti-CTLA-4 conjugate, and after administration of the conjugate, illuminating the first tumor with a light wavelength suitable for use with the phthalocyanine dye. Some embodiments of the methods include administering an immune modulatory agent, such as an immune checkpoint inhibitor prior to, concurrent with, or subsequent to the administration of the anti-CTLA-4 conjugate. The provided compositions, combinations, and methods can sensitize resistant or non-responsive tumors, and/or cold tumors, including primary cold tumors and metastatic cold tumors, to immune modulatory agents.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS OF TREATMENT WITH AND USES OF ANTI-CTLA-4 CONJUGATES

In some embodiments, the methods involve administering an anti-CTLA-4 conjugate and illumination of a tumor or the microenvironment of a tumor (also referred to as tumor microenvironment; TME), or cells that are present in the TME, with a wavelength of light suitable for use with the phthalocyanine dye, such that the light excites the dye and results in cell killing. Such methods result in reducing or eliminating a lesion (e.g., tumor), reducing or inhibiting tumor growth, reducing, inhibiting or eliminating tumor cell invasion, reducing, inhibiting, or eliminating tumor cell metastasis, reducing, inhibiting, or eliminating invasive tumor cells, reducing, inhibiting or eliminating metastatic tumor cells or any combination thereof. In some embodiments, provided are methods for using and uses of the compositions containing a phthalocyanine dye-targeting molecule conjugate, in which the targeting molecule binds CTLA-4 (e.g., an anti-CTLA-4 antibody-IR700 conjugate), for therapy or treatment of a cancer, such as a cancer that includes a first tumor or multiple first tumors (e.g., one or more primary tumors), and the first tumor(s) is/are treated with illumination to effect photoimmunotherapy in the first tumor(s), as well as a secondary population of cancer cells, such as metastatic tumor cells (e.g., metastatic cancers), invasive tumor cells, (e.g., invasive cancers), infiltrating tumor cells (e.g., infiltrating cancers), and/or one or more second tumors or lesions. In some embodiments, the secondary population of cancer cells is related to, such as directly or indirectly derived from, the first tumor. In some embodiments, the secondary population of cells is not directly derived from or related to the first tumor.

In some embodiments of the methods, the growth of the first tumor (e.g., a primary tumor) is inhibited, the volume of the first tumor is reduced or both tumor growth and volume are reduced. In some embodiments of the methods, the growth of the first tumor is inhibited, the volume of the first tumor or first tumors is reduced or both tumor growth and volume are reduced as compared to a monotherapy such as administration of only the conjugate, only the conjugate followed by illumination, or only the immune modulating agent, such as an immune checkpoint inhibitor (e.g., anti-PD-1 antibody).

In some aspects, the methods provoke an immune response in a subject. In some embodiments, the provoked immune response is a systemic immune response. In some embodiments, the provoked immune response is localized to the region of treatment (e.g., a local immune response). In some embodiments, the provided methods provoke a local and systemic immune response. In some aspects the immune response provoked by the provided methods is

In some aspects, the methods also involve administration of an immune modulating agent, such as an immune checkpoint inhibitor (e.g., anti-PD-1 antibody or antigen-binding fragment thereof), in combination with the phthalocyanine dye-targeting molecule conjugate. In some aspects, a combination of a phthalocyanine dye-targeting molecule conjugate and the immune checkpoint inhibitor (e.g., anti-PD-1 antibody or antigen-binding fragment thereof) are employed in the provided methods and uses, such as in the provided methods and uses for treatment of a cancer. In some embodiments, administration of the anti-CTLA-4 antibody-IR700 conjugate, followed by illumination (e.g., anti-CTLA-4-IR700 PIT) sensitizes the treated (illuminated) tumor to anti-PD-1 therapy. In some embodiments, administration of the anti-CTLA-4 antibody-IR700 conjugate, followed by illumination (e.g., anti-CTLA-4-IR700 PIT). In some embodiments, administration of the anti-CTLA-4 antibody-IR700 conjugate, followed by illumination (e.g., anti-CTLA-4-IR700 PIT) sensitizes the treated (illuminated) tumor and one or more distal (non-illuminated) tumors to anti-PD-1 therapy. In any of such embodiments, the anti-CTLA-4 PIT and anti-PD-1 therapy are synergistic.

Uses include uses of the compositions and combinations in such methods and treatments and uses of such compositions and combinations in the preparation of a medicament in order to carry out such therapeutic methods. In some embodiments, the methods and uses thereby treat the cancer, such as cancers that include tumors and cancers that include a first tumor, that is a primary or non-primary tumor, and one or more secondary population(s) of tumor cells (e.g., metastatic tumor cells and/or invasive tumor cells), such as metastatic and/or invasive cancers, in a subject. In some embodiments the secondary tumor cells are related to the first tumor. In some embodiments of the methods and uses more than one tumor is treated. In some aspects, also provided are methods and uses of such compositions and combinations in enhancing, boosting, augmenting, strengthening, increasing, boosting or supporting the immune function, such as systemic immunity, in the subject.

Anti-CTLA-4 antibodies have been used to treat cancers with limited success. The naked CTLA-4 antibodies are thought to generally induce anti-cancer activity through activation of CD8 T cells by checkpoint inhibition. These antibodies, on their own, do not deplete regulatory T cells (Tregs). Their effect is mainly through checkpoint inhibition (similar to PD-1 and PD-L1 inhibitors).

The compositions of anti-CTLA-4 conjugates herein, e.g., comprising an anti-CTLA-4 antibody or antigen-binding fragment thereof conjugated to a phthalocyanine dye, work through a different mechanism. Like the naked antibody molecules, these conjugates can activate CD8⁺ T cells. However, in conjunction with illumination, the anti-CTLA-4 conjugates can deplete intratumoral Treg cells, a functionality not present in the treatment with naked, unconjugated CTLA-4 antibodies.

Provided herein are methods and compositions that include an anti-CTLA-4 conjugate. Such compositions and methods can provide effective treatment in circumstances where Treg depletion increases the effectiveness of the treatment on the tumor or tumor microenvironment (TME). In some cases, the depletion of the Treg cells in the tumor or TME results in necrosis of the tumor cells. In some embodiments Treg depletion occurs in the tumor but not systemically.

In some embodiments, the methods and compositions herein are effective for treating cold tumors such as tumors or cells in the TME that have an immunosuppressive phenotype with a reduced numbers and/or activities or absent of CD8⁺ T effector cells, and/or increased numbers and/or activities of immune suppressor cells such as regulatory T cells (Tregs), myeloid derived suppressor cells, M2 macrophages, tumor associated fibroblasts, or combination thereof. The anti-CTLA4 conjugates following illumination can eliminate immunosuppressive Tregs and myeloid-derived suppressor cells (MDSCs), and as a result, growth of the tumor is reduced or inhibited.

In some embodiments, the methods and compositions herein are effective in treating tumors that exhibit less immunoresponsiveness, for example, tumors that are not responsive to immunotherapy (such as reduced responsiveness to immune checkpoint blockade), tumors that contain high levels of immunosuppressive cell types (e.g., regulatory T cells), a tumor that contains low levels of cytotoxic immune cells (e.g., CD8+ T cells and/or natural killer cells), or any combination thereof. In some embodiments, the methods and compositions herein are effective for treating tumors that are larger in size, and which exhibit greater immune suppression, e.g., contain increased levels of regulatory T cells, than smaller tumors. Such tumors may be less responsive or non-responsive to other treatments, such as to treatment with an immune checkpoint inhibitor (e.g., a naked CTLA-4 antibody, a naked PD-1 antibody, or a naked PD-L1 antibody), or treatment with other antibodies or antibody-conjugates. The effectiveness of the anti-CTLA-4-IR700 PIT to inhibit or substantially reduce growth of larger tumors differentiates it from treatment with naked anti-CTLA4 antibody or conjugate alone, which does not provide the inhibition or reduction in growth of these tumors.

A. Methods and Compositions for Treating a Tumor or Tumor Cells that are not Responsive to Prior Therapeutic Treatments

In some embodiments, provided are methods and compositions containing an anti-CTLA-4 conjugate, e.g., a phthalocyanine dye-targeting molecule conjugate, in which the targeting molecule binds to CTLA-4 (e.g., an anti-CTLA-4 antibody-IR700 conjugate), for therapy or treatment of a cancer that has failed to or is not responsive to one or more prior treatments with an immune checkpoint inhibitor, an anticancer agent, and/or therapeutic agent against immune suppressor cells. The cancers include a first tumor or multiple first tumors as well as metastatic tumor cells, for example metastatic cancers; or a cancer that includes a first tumor or multiple first tumors as well as invasive or metastatic tumor cells, for example, invasive cancers or metastatic cancers.

Such methods and uses include, for example, administration of an CTLA-4 conjugate (e.g., an anti-CTLA-4 antibody or antigen-binding fragment thereof conjugated to a phthalocyanine dye) to a subject having a tumor or tumor cells followed by illumination of at the site of a tumor (such as a first tumor or primary tumor) or tumorous cells or the microenvironment of a tumor, using a suitable light wavelength for the phthalocyanine dye and light dose. In some aspects, the illumination results in an illumination-dependent lysis and death of cells expressing the target molecule (e.g., CTLA-4), resulting in a therapeutic effect or treatment of the cancer. In some cases, cells within the TME expressing CTLA-4 are killed and thus rapidly depleted from the TME (such as Treg cells), and as a result, necrosis of the tumor cells can occur.

In some aspects, the methods also involve the administration of an immune modulating agent, such as an immune checkpoint inhibitor, in combination with an anti-CTLA-4 conjugate. In some aspects, such combination is employed for treatment of a cancer, a tumor or a cancerous lesion. In some embodiments, the methods include the administration of the immune modulating agent, such as an immune checkpoint inhibitor, prior to, concurrent with or subsequent to the administration of an anti-CTLA-4 conjugate (e.g., an anti-CTLA-4 antibody or antigen-binding fragment thereof conjugated to a phthalocyanine dye). In such methods, the primary tumors, invasive tumor cells, and metastatic tumor cells can be sensitized to the treatment with an immune modulatory agent. In such methods, the growth of primary tumors, invasive tumor cells, and metastatic tumor cells can be inhibited, reduced or eliminated, and/or the volume of one or more tumors is reduced.

The increase in sensitivity as a result of such combination treatments can include, but not limited to, a reduction of inhibition of tumor growth, a reduction in tumor cell invasion and/or metastasis, an increase in tumor cell killing, an increase in systemic immune response, an increase in new T cell priming, an increase in the diversity of intratumoral CD8⁺ T cells, an increase in the number and/or activity of intratumoral CD8⁺ T effector cells, a decrease in the number and/or activity of intratumoral regulatory T cells, a decrease in the number and/or activity of intratumoral myeloid derived suppressor cells, a decrease in the number and/or activity of intratumoral tumor associated fibroblasts, or any combination thereof.

In some aspects, the prior therapeutic treatment or treatments to which a cancer, tumor, or tumor cells are not responsive can be treatment with an immune checkpoint inhibitor. The prior immune checkpoint inhibitor can be a PD-1 inhibitor, a PD-L1 inhibitor, or combination thereof. The prior immune checkpoint inhibitor can be a small molecule inhibitor, an antibody inhibitor, or other molecule that binds to and inhibits an immune checkpoint protein. In some aspects, the prior immune checkpoint inhibitor can be an anti-PD-1 antibody or an antigen-binding fragment thereof. In some aspects, the prior immune checkpoint inhibitor can be an anti-PD-L1 antibody or an antigen-binding fragment thereof. For example, an antibody inhibitor for PD-1 can include, but are not limited to, any of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, TSR-042 (ANB011). An antibody inhibitor for PD-L1 can include, for example, but are not limited to, any of atezolizumab (MPDL3280A, TECENTRIQ, RG7446), avelumab (BAVENCIO, MSB0010718C; M7824), durvalumab (MEDI4736, IMFINZI), LDP, NM-01, STI-3031 (IMC-001; STI-A1015), KN035, LY3300054, M7824 (MSB0011359C), BMS-936559, MSB2311, BCD-135, BGB-A333, CBT-502 (TQB-2450), cosibelimab (CK-301), CS1001 (WPB3155), FAZ053, MDX-1105, SHR-1316 (HTI-1088), TG-1501, ZKAB001 (STI-A1014), INBRX-105, MCLA-145, KN046, LY3415244, REGN3504, HLX20.

In some aspects, the prior therapeutic treatment or treatments to which a cancer, tumor or tumor cells are not responsive can be treatment with an immunomodulating agent such as a cytokine, for example, Aldesleukin (PROLEUKIN), Interferon alfa-2a, Interferon alfa-2b (Intron A), Peginterferon Alfa-2b (SYLATRON/PEG-Intron), or a cytokine that targets the IFNAR1/2 pathway, the IL-2/IL-2R pathway, or such as an adjuvant, for example, Poly ICLC (HILTONOL/Imiquimod), 4-1BB (CD137; TNFRS9), OX40 (CD134) OX40-Ligand (OX40L), Toll-Like Receptor 2 Agonist SUP3, Toll-Like Receptor TLR3 and TLR4 agonists and adjuvants targeting the Toll-like receptor 7 (TLR7) pathway, other members of the TNFR and TNF superfamilies, other TLR2 agonists, TLR3 agonists and TLR4 agonists.

In some aspects, the prior therapeutic treatment or treatments to which the cancers are not responsive include using an anticancer agent. The prior anticancer agent can be one or more of a chemotherapeutic agent, an antibody treatment, and/or a radiotherapeutic agent.

In some aspects, the prior therapeutic treatment or treatments to which the cancers are not responsive include using a therapeutic agent targeted against immune suppressor cells. The agent can be an antibody, for example, anti-CD25 antibodies that target regulatory T cells; a small molecule inhibitor or combination thereof. Immune suppressor cells include regulatory T cells, M2 macrophages, tumor associated fibroblasts, or combination thereof.

B. Tumors and Tumor Cell Targets for Anti-CTLA-4 Conjugate Therapy and Anti-CTLA-4 Conjugate Combination Therapy

The methods described herein include administration of an anti-CTLA-4 conjugate (e.g., an anti-CTLA-4 antibody or antigen-binding fragment thereof conjugated to a phthalocyanine dye) and illuminating a first tumor or lesion or the site of the first tumor or lesion or the tumor microenvironment (TME) of a first tumor or lesion, in a subject with a wavelength of light to activate the phthalocyanine dye moiety of the conjugate to achieve cell killing, e.g., targeted killing of cells expressing CTLA-4. In some embodiments, the methods and uses provided herein include treating a subject that has one or more first tumors. The subject may have one, two, three, or more than three first (e.g., primary) tumors. Such tumors can be in one or more tissues or organs, such as in one tissue or organ, in two different tissues or organs, in three different tissues or organs, or in more than three different tissues or organs.

In some aspects, a first tumor can refer to the first, primary or original tumor in a subject; a first tumor can also refer to the one or more tumors selected for illumination with the methods and uses provided herein. In some embodiments first tumor is synonymous with primary tumor. In some embodiments, a first tumor or first tumors may be a solid tumor or solid tumors, may be lymphomas, or may be leukemias. The tumor can be tumor of the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, or unknown primary origin.

In some embodiments, the methods and uses provided herein include treating a subject that has a first tumor and also a secondary population of tumor cells, such as invasive and/or metastatic tumor cells, or one or more second tumors. The methods include administering to a subject having a first tumor and a secondary population of related tumor cells, such as invasive and/or metastatic tumor cells, a conjugate comprising a phthalocyanine dye linked to targeting molecule, wherein the targeting molecule binds to CTLA-4 and after administration of the conjugate, illuminating the first tumor with a wavelength suitable for the selected phthalocyanine dye. In some of such embodiments, the secondary population of tumor cells is not directly illuminated. In some embodiments of the methods herein with anti-CLTA-4 conjugate and illumination (anti-CTLA-4 PIT) treatments (alone or in combination(s)), the growth of a primary/first (illuminated) tumor or additional (non-illuminated) tumors is inhibited; the size (e.g., volume, dimension(s), or mass) of the first (illuminated) tumor or additional tumors is reduced; or both tumor growth and size (e.g., volume, dimension(s), or mass) are inhibited or reduced.

In some embodiments, the methods include the administration of an immune checkpoint inhibitor, such as an anti-PD-1 antibody, prior to, concurrent with or subsequent to the administration of the conjugate. In such methods, the growth (volume, dimension, or mass) of the first tumor and/or the secondary population of tumor cells or one or more second tumor(s), such as metastatic tumor cells, is inhibited, reduced or eliminated, the volume, dimension, or mass of one or more of the first tumor and/or secondary population of cells or one or more second tumor(s) is reduced, or any combination thereof. In some embodiments, the inhibition of the first tumor and/or secondary population or one or more second tumor(s) is effected to a greater degree than the inhibition achieved by administration of only the conjugate, only the conjugate followed by illumination, or only the anti-PD-1 antibody. In some embodiments, inhibition is achieved if the tumor exhibits less than 20% increase in tumor volume, tumor dimension(s), or mass; no change in tumor volume, dimension, or mass (i.e., halted tumor growth or progression); or the tumor is reduced in volume, dimensions, or mass; or there is a reduction in number of tumor cells.

In any of the methods and uses herein, the first tumor can be a primary tumor or a secondary tumor. In some embodiments, the first tumor and the secondary population are related. In some embodiments, the secondary population of cells is directly or indirectly derived from the first tumor. In some embodiments, the secondary population of cells comprises one or more second tumor(s) or second lesion(s). In some embodiments, the secondary population is not derived from the first tumor. In some embodiments, the first tumor is a primary tumor and the secondary population of cells is related to the primary tumor; for example, the secondary population of cells is derived directly or indirectly from the primary tumor. In some embodiments, the first tumor is a primary tumor and the secondary population of tumor cells is a second primary tumor. In some embodiments, the first tumor is a secondary tumor and the secondary population of cells is related to the secondary tumor. In some aspects, a secondary population of tumor includes cells that originated from a primary tumor and invade local or distal healthy tissue (i.e., invasive tumor cells) or spread to a distal tissue or organ, or distal tissues or organs within the body of a subject having the primary tumor (i.e., metastatic tumor cells), for example a tissue or an organ located distantly or far away from the primary tumor. In some aspects a secondary population of tumor cells is both invasive and metastatic. In some aspects a secondary population of tumor cells is infiltrating. In some aspects, a secondary population of tumor cells is metastatic and is directly or indirectly related to, such as derived from, the first tumor. In other aspects, a secondary population of tumor cells is metastatic and not directly related to the first tumor. Metastatic tumor cells can be located in one or more locations in the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, blood, bone marrow, cerebrospinal fluid, or any other tissues or organs. In some embodiments, metastatic tumor cells are contained in a solid tumor. In some embodiments, metastatic tumor cells are circulating tumor cells, are a liquid tumor, or are not associated with a tumor mass.

In some embodiments, the methods and uses provided herein include treating a subject that has one or more first tumor(s) and also invasive or metastatic tumor cells, such as when cells originating from a first tumor have invaded into surrounding tissues. In some embodiments, the methods and uses provided herein include treating a subject that has one or more first tumor(s) and also one or more second tumor(s) or lesion(s) In some embodiments, the first tumor is a primary tumor, and the invasive tumor cells are directly or indirectly derived from the first tumor. In some embodiments, the invasive tumor cells are not directly derived from the first tumor. The methods include administering to a subject having a first tumor(s) and invasive tumor cells, an anti-CTLA-4 conjugate and after administration of the conjugate, illuminating the first tumor with a wavelength suitable for the selected phthalocyanine dye. In some embodiments, the methods include the administration of an immune modulatory agents, such as an immune checkpoint inhibitor, prior to, concurrent with, or subsequent to the administration of the conjugate.

In some aspects, invasive tumor cells refer to cells originated from a first tumor and have invaded into surrounding tissues of the same organ or neighboring organs or body cavities of the first tumor within the body of a subject having the first tumor.

In some instances, the methods provided herein include illumination of the one or more first tumors, and some or all of the invasive or metastatic tumor cells, or second tumors or second lesions, are not illuminated, and in such methods, the growth of invasive or metastatic tumor cells is inhibited, reduced or eliminated; the growth of a second tumor is inhibited, reduced, or eliminated; the volume, dimension(s), or mass of one or more invasive or metastatic tumors is reduced; the volume, dimension(s), or mass of one or more second tumor(s) is reduced; or any combination thereof. In some embodiments, the growth of the first tumor also is inhibited, reduced or eliminated. For example, the growth or volume of one or more first tumors is reduced along with the effect(s) on the one or more invasive or metastatic tumor cells and/or the effects on one or more second tumor(s).

In some embodiments, invasive tumor cells are contained in a solid tumor. In some embodiments, invasive tumor cells are contained in body fluids, including but not limited to peritoneal fluid, pleural fluid, and cerebrospinal fluid. In some embodiments, invasive tumor cells are contained in the effusion of a body cavity or body cavities, including but not limited to peritoneal effusion (ascites), pleural effusion, and pericardial effusion.

In some embodiments, the methods and uses provided herein include treating a subject that has one or more first tumors and also metastatic tumor cells. The methods include administering to a subject having first tumor(s) and metastatic tumor cells, an anti-CTLA-4 conjugate and after administration of the conjugate, illuminating the first tumor with a wavelength suitable for the selected phthalocyanine dye. In some embodiments, the methods include the administration of an immune modulatory agent, such as an immune checkpoint inhibitor prior to, concurrent with or subsequent to the administration of the conjugate. In such methods, the growth of metastatic tumor cells is inhibited, reduced or eliminated, the volume of one or more metastatic tumors is reduced or any combination thereof.

In some embodiments of the methods and uses provided herein, metastatic tumor cells are distal to the first tumor and some or all of the metastatic tumor cells are not illuminated, e.g., not directly illuminated. In some embodiments of the methods and uses, only the one or more first tumor is illuminated after administration of the conjugate and metastatic tumor cells are not directly illuminated. In some embodiments, more than one first tumor is illuminated but at least one site of tumor cells, such as containing metastatic tumor cells, is not illuminated.

In some aspects, metastatic tumor cells include cells originated from a first tumor and spread to distal tissue or organ, or distal tissues or organs within the body of a subject having the first tumor. The metastatic tumor cells can be located in one or more locations in the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, blood, bone marrow, cerebrospinal fluid, or any other tissues or organs. In some embodiments, metastatic tumor cells are contained in a solid tumor. In some embodiments, metastatic tumor cells are circulating tumor cells or are not associated with a tumor mass.

### II. METHODS FOR PROVOKING OR ENHANCING LOCAL AND SYSTEMIC IMMUNE RESPONSES

In some embodiments herein, the methods herein with compositions including an anti-CTLA-4 conjugate can result in an enhancement of the systemic and/or local response in the subject, which in turn can result in an enhanced or synergistic response to the therapy or treatment for cancer or a tumor. In some aspects the cancer or tumor to be treated exhibits reduced immunoresponsiveness (e.g., contains one or more “cold” tumor(s)). In some embodiments, a tumor that exhibits less immunoresponsiveness is a tumor that characterized by not being responsive to an immunotherapy (such as reduced responsiveness to treatment with an immune checkpoint inhibitor), containing high levels of immunosuppressive cell types (e.g., regulatory T cells), containing low levels of cytotoxic effector immune cells (e.g., CD8⁺ T cells and/or natural killer cells), or any combination thereof. In some embodiments, the methods and compositions herein are effective for treating tumors that are larger in size, and which exhibit greater immune suppression, e.g., contain increased levels of regulatory T cells, or reduced immune cell infiltration, such as reduced cytotoxic effector immune cell infiltration, than smaller tumors. Such tumors may be less responsive or non-responsive to other treatments, such as to treatment with an immune checkpoint inhibitor (e.g., a naked (unconjugated) CTLA-4 antibody, a naked PD-1 antibody, or a naked PD-L1 antibody), or treatment with other antibodies or antibody-conjugates. The effectiveness of the anti-CTLA-4-IR700 PIT to inhibit or substantially reduce growth of larger tumors, or tumors that contain reduced cytotoxic immune cell infiltrate, differentiates it from treatment with naked anti-CTLA4 antibody or conjugate alone, which does not provide the inhibition or reduction in growth of these tumors.

In some aspects, the methods and uses herein include administering to the subject anti-CTLA-4 conjugate, administering an immune modulatory agent, such an immune checkpoint inhibitor, and after administration of the conjugate, illuminating the tumor or a cancerous lesion, or the tumor microenvironment. The immune modulatory agent can be administered prior to, concurrent with or subsequent to the administration of the conjugate. Combination therapies include those further described in Section IV.

In some embodiments, the methods and compositions herein provoke, stimulate, boost, augment, or support an immune response, such as a systemic response, such as a systemic immune response, in a subject having a cancer. In some embodiments, the method and uses herein includes enhancing a systemic immune response in a subject having a cancer, a tumor or a cancerous lesion. “Systemic immune response” refers to the ability of a subject's immune system to respond to an immunologic challenge or immunologic challenges, including those associated with a cancer, a tumor, or a cancerous lesion, in a systemic manner. Systemic immune response can include systemic response of the subject's adaptive immune system and/or innate immune system. In some aspects, systemic immune response includes an immune response across different tissues, including the blood stream, lymph node, bone marrow, spleen and/or the tumor microenvironment, and in some cases, includes a coordinated response among the tissues and organs and various cells and factors of the tissues and organs. Also provided herein are methods and uses of compositions and combinations in enhancing, boosting or augmenting the response to a treatment or a therapy in a subject, such as in a subject having a cancer or a tumor.

In some aspects, the methods and compositions provided herein can also exhibit an abscopal effect. In some aspects, “abscopal effect” refers to a treatment effect in which a tumor that is not directly illuminated or is away from the site of the localized illumination, e.g., a distal or a metastatic tumor, is also treated, for example, resulting in reduced in tumor volume, dimension(s), and/or mass.

In some aspects, the provide methods and uses include administering to the subject a conjugate comprising a phthalocyanine dye linked to targeting molecule, wherein the targeting molecule binds to CTLA-4, and after administration of the conjugate, illuminating the tumor or a cancerous lesion, or the tumor microenvironment, and administering an immune checkpoint inhibitor. The conditions for illumination regarding wavelength, dosage of illumination and timing of illumination are such as those described herein. The immune checkpoint inhibitor can be administered prior to, concurrent with or subsequent to the administration of the conjugate, such as described herein. In some aspects, the methods and uses provided herein result in an enhancement of the systemic and/or local immunity in the subject, which can in turn result in enhanced or synergistic response to the therapy or treatment for cancer. In some embodiments, the methods and uses provided herein results in an enhanced response, such as a synergistic response, to the treatment or therapy for the cancer or the tumor, as compared with the administration of only the conjugate, or only the immune checkpoint inhibitor. For example, in some embodiments the provided methods and uses result in a synergistic response with the combination of anti-CTLA-4, PIT and an immune checkpoint inhibitor, such as an anti-PD-1 antibody, that is more effective in treating a first, target tumor and/or a second tumor cell, distal to the illuminated first tumor, than only anti-CTLA-4 PIT or only the anti-PD-1 antibody. In some aspects, the enhanced response comprises an enhancement of systemic and/or local immunity of the subject as compared to the systemic and/or local immunity of the subject prior to the administration of the anti-CTLA-4 conjugate followed by illumination (anti-CTLA-4 PIT) and the immune checkpoint inhibitor (e.g., the anti-PD-1 antibody). In some aspects, the enhanced response comprises an enhanced response, such as an additional, additive, or synergistic response, to the treatment as compared with administration of only the anti-CLTLA-4 conjugate, only the anti-CTLA-4 conjugate followed by illumination, or only the immune checkpoint inhibitor (e.g., the anti-PD-1 antibody).

The methods and combinations herein can provoke, increase, or augment a systemic response, such as a systemic immune response, against a first tumor, invasive tumor, a metastatic tumor and/or invasive or metastatic tumor cells. In some embodiments, the first tumor is a “cold” tumor that exhibits reduced immunoresponsiveness. In some embodiments, the invasive tumor or metastatic tumor is a “cold” tumor that exhibits reduced immunoresponsiveness. In some embodiments, the first tumor and the invasive tumor and/or metastatic tumor are “cold” tumors, each exhibiting reduced immunoresponsiveness.

In some aspects, the provoked or increased systemic immune response includes an increase in the number and/or activity of systemic CD8⁺ T effector cells, an increase in systemic T cell cytotoxicity against tumor cells as measured using a CTL assay using cells from the spleen, the peripheral blood, the bone marrow, or the lymph nodes, an increase in the number and/or activity of intratumoral CD8⁺ T effector cells in the invasive tumors and/or metastatic tumors, an increase in systemic CD8⁺ T cell activation, an increase in the CD8⁺:Treg ratio in the invasive and/or metastatic tumors, an increase in natural killer cell infiltration in the invasive and/or metastatic tumors, and increase in natural killer cell activation in the invasive and/or metastatic tumors, an increase in systemic dendritic cell activation, an increase in dendritic cell activation in the invasive tumors and/or metastatic tumors, an increase in intratumoral dendritic cell infiltration in the invasive tumors and/or metastatic tumors, an increase in new T cell priming in the invasive tumors and/or metastatic tumors, an increase in T cell diversity in the invasive tumors and/or metastatic tumors, a decrease in systemic regulatory T cells, a decrease in regulatory T cells in the invasive tumors and/or metastatic tumors, a decrease in systemic myeloid derived suppressor cells, a decrease in intratumoral myeloid derived suppressor cells in the invasive tumors and/or metastatic tumors, a decrease in tumor associated fibroblasts in the invasive tumors and/or metastatic tumors, or any combination thereof in the subject. In some instances, a systemic response can be assessed by sampling blood, tissue, cells or other fluid from a subject and assessing an increase in proinflammatory cytokines, an increase or appearance of immune cell activation markers and/or T cell diversity.

In some aspects, the level, strength or extent of systemic immunity can be measured based on the number of intratumoral CD8⁺ T lymphocytes, the ratio of CD8⁺ T lymphocytes to regulatory T cells (Tregs), intratumoral T lymphocyte exhaustion (e.g., the percentage of CD3⁺CD8⁺ cells that express PD-1 and/or CTLA4 markers), the number or percentage of intratumoral activated CD8⁺ T lymphocytes (e.g., Ki67⁺ or CD69⁺ CD8 cells as a percentage of CD45⁺ cells), the expansion of cytotoxic intratumoral T lymphocytes (e.g., the percentage of CD3⁺ CD8⁺ cells that do not express PD-1 and/or CTLA4 markers), based on the splenocyte cytotoxicity against tumor cells, or any or all of combination thereof. In some aspects, intratumoral CD8⁺ T lymphocytes include CD3⁺ CD8⁺ cells, intratumoral exhausted T lymphocytes include PD-1⁺CTLA-4⁺CD3⁺ CD8⁺ cells, activated intratumoral CD8⁺ T lymphocytes include CD3⁺CD8⁺Ki67⁺ and/or CD3⁺ CD8⁺CD69⁺ cells, expansion of cytotoxic T lymphocytes include PD-1⁻CTLA-4⁻CD3⁺ CD8⁺ cells. In some aspects, intratumoral CD8⁺ T lymphocytes, exhausted intratumoral T lymphocytes, activated CD8⁺ T lymphocytes, or expanded cytotoxic intratumoral T lymphocytes are measured as a percentage of leukocytes (CD45⁺ cells) and/or total CD8⁺ T cells (e.g., CD3⁺ CD8⁺CD45⁺ cells).

In some embodiments, the level, strength or extent of systemic immunity can be measured based on the number or percentage of intratumoral natural killer cells, the number or percentage of intratumoral activated natural killer cells (e.g., CD49b⁺ CD3⁻Ki67⁺⁻ cells as a percentage of CD45⁺ cells, or CD49b⁺ CD3⁻CD69⁺ cells as a percentage of CD45⁺ cells). Determination of such numbers or percentages can be achieved using several well-known methods, including those described herein. For example, such numbers or percentages can be determined by generating single cell suspensions, such as by mechanical dissociation of tumor and/or tissue biopsies, or collection of blood samples containing circulating immune cells, followed by staining and flow cytometric analysis or mass cytometry. Other methods can include multiplexed immunofluorescence imaging of tissue and/or tumor biopsies.

In some embodiments, the strength or extent of systemic immunity is measured by the number of intratumoral CD8⁺ T lymphocytes, such as CD3⁺ CD8⁺ T lymphocytes, and the systemic immunity against recurring tumors is increased or augmented if the percentage of intratumoral CD8⁺ T lymphocytes (e.g., CD3⁺ CD8⁺ T lymphocytes), among the total number of CD45⁺ cells, is increased after treatment as compared to before treatment. In some of such examples, the systemic immunity against recurring tumors is increased or augmented if the number of intratumoral CD8⁺ T lymphocytes (e.g., CD3⁺ CD8⁺ T lymphocytes) is at least at or about 30% of the total number of CD45⁺ cells, such as at least at or about 30%, 35%, 36%, 37%, 38% 39%, 40%, 41%, 42% 43%, 44%, 45%, 46%, 47%, 48%, 49%, 50%, 51%, 52%, 53%, 54%, 55%, 56%, 57%, 58%, 59%, 60%, or more of the total number of CD45⁺ cells. In some embodiments, the percentage of intratumoral CD8⁺ T lymphocytes is at least 40% of the intratumoral CD45⁺ cell population. In some embodiments, the systemic immunity against recurring tumors is increased or augmented if the percentage of intratumoral CD3⁺ CD8⁺ T cells among the population of intratumoral CD45⁺ cells is increased after treatment as compared to before treatment. In some of such embodiments the percentage of intratumoral CD3⁺ CD8⁺ T cells among the population of intratumoral CD45⁺ cells is increased after treatment by at least at or about 5%, 6%, 7%, 8%, 9%, 10%, 11%, 12%, 13%, 14%, 15%, 16%, 17%, 18%, 19%, 20%, 25%, 30%, or more compared to before treatment. In some embodiments, the percentage of intratumoral CD3⁺ CD8⁺ T cells among the population of intratumoral CD45⁺ cells is increased after treatment by at least 10% as compared to before treatment.

In some embodiments, the strength or extent of systemic immunity is measured by the number of exhausted intratumoral CD8⁺ T lymphocytes, such as the number of PD-1⁺CTLA-4⁺CD3⁺ CD8⁺ cells as a percentage of intratumoral CD8⁺ T lymphocytes (e.g., CD3⁺ CD8⁺ T lymphocytes), and the systemic immunity against recurring tumors is increased or augmented if the percentage of PD-1⁺CTLA-4⁺CD3⁺ CD8⁺ cells among intratumoral CD3⁺ CD8⁺ T cells is decreased after treatment as comparted to before treatment. In some of such examples, the systemic immunity against recurring tumors is increased if the percentage of PD-1⁺CTLA-4⁺CD3⁺ CD8⁺ cells among intratumoral CD3⁺ CD8⁺ T cells after treatment is less than at or about 20%, such as less than 19%, 18%, 17%, 16%, 15%, 14%, 13%, 12%, 11%, 10%, 9%, 8%, 7%, 6%, 5%, 4%, 3%, 2%, 1%, or less. In some embodiments, the systemic immunity against recurring tumors is increased or augmented if the percentage of PD-1⁺CTLA-4⁺CD3⁺ CD8⁺ cells among intratumoral CD3⁺ CD8⁺ T cells is reduced after treatment compared to before treatment. In some of such embodiments, the percentage of PD-1⁺CTLA-4⁺CD3⁺ CD8⁺ cells among intratumoral CD3⁺ CD8⁺ T cells is decreased by at least at or about 5%, 6%, 7%, 8%, 9%, 10%, 11%, 12%, 13%, 14%, 15%, 16%, 17%, 18%, 19%, 20%, 25%, 30%, or more compared to before treatment. In some embodiments, the percentage of exhausted intratumoral CD8⁺ T cells (PD-1⁺CTLA-4⁺CD3⁺CD8⁺ cells) among the population of intratumoral CD3⁺ CD8⁺ T cells is decreased after treatment by at least 10% as compared to before treatment.

In some embodiments, the strength or extent of systemic immunity is measured by the number of activated intratumoral CD8⁺ T lymphocytes, such as CD3⁺ CD8⁺Ki67⁺ and/or CD3⁺ CD8⁺CD69⁺ T lymphocytes, and the systemic immunity against recurring tumors is increased or augmented if the number of activated intratumoral CD8⁺ T lymphocytes, such as CD3⁺ CD8⁺Ki67⁺ and/or CD3⁺ CD8⁺CD69⁺ T lymphocytes, as a percentage of intratumoral CD45⁺ leukocytes, is increased after treatment as compared to before treatment. In some of such embodiments, the systemic immunity against recurring tumors is increased or augmented if the number of intratumoral CD3⁺ CD8⁺Ki67⁺ cells, is at least at or about 0.15% of the total number of CD45⁺ cells, such as at least at or about 0.2%, 0.25%, 0.3%, 0.35%. 0.4%, 0.45%, 0.5%, or more of the total number of intratumoral CD45⁺ cells after treatment. In other of such embodiments, the systemic immunity against recurring tumors is increased or augmented if the number of intratumoral CD3⁺ CD8⁺CD69⁺ cells, is at least at or about 0.5% of the total number of CD45⁺ cells, such as at least at or about 0.6%, 0.7%, 0.8%, 0.9%. 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, or more of the total number of intratumoral CD45⁺ cells, such as at least about 1.0% of the total number of intratumoral CD45⁺ cells after treatment. In some embodiments, the systemic immunity against recurring tumors is increased or augmented if the percentage of intratumoral CD3⁺ CD8⁺Ki67⁺ and/or CD3⁺ CD8⁺CD69⁺ T lymphocytes among intratumoral CD45⁺ cells is increased after treatment compared to before treatment. In some of such embodiments, the percentage of CD3⁺ CD8⁺Ki67⁺ and/or CD3⁺ CD8⁺CD69⁺ T lymphocytes cells among intratumoral CD45⁺ cells is increased by at least at or about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold-18-fold, 19-fold, 20-fold, 21-fold, 22-fold, 23-fold, 24-fold, 25-fold, 30-fold, 35-fold, 40-fold, 45-fold, 50-fold, 55-fold, 60-fold, or more compared to the percentage of CD3⁺CD8⁺Ki67⁺ and/or CD3⁺ CD8⁺CD69⁺ T lymphocytes cells among intratumoral CD45⁺ cells before treatment. In some embodiments, the percentage of intratumoral CD3⁺ CD8⁺Ki67⁺ T lymphocytes cells among intratumoral CD45⁺ cells is increased by at least 15-fold or 20-fold compared to the percentage of CD3⁺ CD8⁺Ki67⁺ T lymphocytes cells among intratumoral CD45⁺ cells before treatment. In some embodiments, the percentage of intratumoral CD3⁺CD8⁺CD69⁺ T lymphocytes cells among intratumoral CD45⁺ cells is increased by at least 5-fold compared to the percentage of CD3⁺ CD8⁺CD69⁺ T lymphocytes cells among intratumoral CD45⁺ cells before treatment.

In some embodiments, the strength or extent of systemic immunity is measured by the expansion of intratumoral cytotoxic T lymphocytes, such as PD-1⁻CTLA-4⁻CD3⁺ CD8⁺ cells, and the systemic immunity against recurring tumors is increased or augmented if the percentage of intratumoral cytotoxic T lymphocytes (e.g., PD-1⁻CTLA-4⁻CD3⁺ CD8⁺ cells) among CD8⁺ T cells (e.g., CD3⁺CD8⁺ T cells) is increased after treatment as compared to before treatment. In some of such examples, the systemic immunity against recurring tumors is increased or augmented if the number of intratumoral cytotoxic T lymphocytes (e.g., PD-1⁻CTLA-4⁻CD3⁺CD8⁺ cells) is at least at or about 20% of the total number of CD3⁺CD8⁺ T cells, such as at least at or about 25%, 30%, 35%, 40%, 41%, 42% 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, or more of the total number of CD45⁺ cells. In some embodiments, the percentage of intratumoral PD-1⁻CTLA-4⁻CD3⁺CD8⁺ cells is at least at or about 40%, 45%, 50%, or 55% of the intratumoral CD3⁺CD8⁺ T cell population. In some embodiments, the systemic immunity against recurring tumors is increased or augmented if the percentage of intratumoral PD-1⁻CTLA-4⁻CD3⁺CD8⁺ cells among the population of intratumoral CD3⁺CD8⁺ T cells is increased after treatment as compared to before treatment. In some of such embodiments the percentage of intratumoral PD-1⁻CTLA-4⁻CD3⁺CD8⁺ cells among the population of intratumoral CD3⁺CD8⁺ T cells is increased after treatment by at least at or about 5%, 10%, 15%, 20%, 25%, 30%, 35%, 40%, 50%, 60%, 70%, 75%, 80%, or more compared to the percentage of intratumoral PD-1⁻CTLA-4⁻CD3⁺CD8⁺ cells among the population of intratumoral CD3⁺CD8⁺ T cells before treatment. In some embodiments, the percentage of intratumoral PD-1⁻CTLA-4⁻CD3⁺CD8⁺ cells among the population of intratumoral CD3⁺CD8⁺ T cells is increased after treatment by at least 30% as compared to the percentage of intratumoral PD-1⁻CTLA-4⁻CD3⁺CD8⁺ cells among the population of intratumoral CD3⁺CD8⁺ T cells before treatment.

In some embodiments, treatment in accordance with the methods and uses provided herein, leads to cell death of or a reduction in number of regulatory T cells (Tregs), such as intratumoral CD4⁺FoxP3⁺ Tregs. Hence, in some embodiments, the level, strength, or extent of systemic immunity can be measured based on the number or percentage of intratumoral Tregs. In some aspects, binding of the anti-CTLA-4 conjugate to the surface of CTLA-4-expressing cells, such as certain Tregs, and illumination to effect illumination-dependent lysis and death of intratumoral cells expressing CTLA-4, results in a reduction of the number of cells expressing CTLA-4. In some aspects, such results lead to a reduction in the number of immunosuppressive cells, such as Tregs, within the tumor, and thus can alleviate or reverse immunosuppression in the tumor. In some aspects, such reduction in immunosuppressive cells can result in the activation and proliferation of intratumoral T cells, such as intratumoral CD8⁺ cytotoxic T cells or CD4⁺ helper T cells, that can eliminate tumor cells, and lead to reduction of tumor volume and/or elimination of the tumor. In some aspects, treatment in accordance with the provided embodiments can result in a reduction of intratumoral Tregs and/or an increase of intratumoral CD8⁺ to Treg ratio or intratumoral CD4⁺ to Treg ratio. In some embodiments of the provided methods and uses, systemic Tregs are not reduced as a result of treatment.

In some aspects, treatment in accordance with the methods and uses provided herein can result in a lasting or durable decrease in intratumoral Tregs. In some aspects, treatment in accordance with the methods and uses provided herein can result in a lasting or durable increase of intratumoral CD8⁺ to Treg ratio or intratumoral CD4⁺ to Treg ratio. In some embodiments, the level, strength or extent of systemic immunity can be measured by determining the intratumoral CD8⁺ to Treg ratio, and the systemic immunity against recurring tumors is increased or augmented if the intratumoral CD8⁺ to Treg ratio is increased after treatment as compared to before treatment. In some of such examples, the systemic immunity against recurring tumors is increased or augmented if the intratumoral CD8⁺ to Treg ratio is increased by at least at or about 1-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold. 4.0-fold, or more compared to the intratumoral CD8⁺ to Treg ratio before treatment. In some embodiments, the level, strength or extent of systemic immunity can be measured by determining the intratumoral CD4⁺ to Treg ratio, and the systemic immunity against recurring tumors is increased or augmented if the intratumoral CD4⁺ to Treg ratio is increased after treatment as compared to before treatment. In some embodiments, the level, strength or extent of systemic immunity can be measured by determining the intratumoral Treg to CD45⁺ ratio, and the systemic immunity against recurring tumors is increased or augmented if the intratumoral Treg to CD45⁺ ratio is decreased after treatment as compared to before treatment. In some aspects, such increases or decreases can last for at or about 3, 4, 5, 6, 7, 8, 9, 10, 11, 12, 13, 14 days or 3, 4, 5, 6, 7 or 8 weeks or longer.

In some aspects, the level, strength or extent of systemic immunity can be measured by a CTL activity assay using splenocytes or peripheral blood cells or bone marrow cells or lymph node cells. In some embodiments, the cells are collected from the subject between day 4 and day 28 after illumination of the first tumor in the subject.

In some aspects, the level, strength or extent of systemic immunity can be measured by an intratumoral T cell exhaustion assay using T cells collected from the first tumor or a metastatic tumor cells mass or an invasive tumor cell mass. In some embodiments, the cells are collected from the subject between day 4 and day 28 after illumination of the first tumor in the subject.

In some aspects, the level, strength or extent of systemic immunity can be measured by an intratumoral effector T cell expansion assay using T cells collected from the first tumor or a metastatic tumor cells mass or an invasive tumor cell mass. In some embodiments, the cells are collected from the subject between day 4 and day 28 after illumination of the first tumor in the subject.

In some aspects, the level, strength or extent of systemic immunity can be measured by a T cell receptor diversity assay using T cells collected from the first tumor or a metastatic tumor cells mass or an invasive tumor cell mass or the peripheral circulation. In some embodiments, the cells are collected from the subject between day 4 and day 28 after illumination of the first tumor in the subject.

In some aspects, the level, strength or extent of systemic immunity can be measured by determining the presence, number or frequency of regulatory T cells (Tregs) in the tumor and/or the ratio of intratumoral Treg cells to intratumoral CD8⁺ T cells or intratumoral CD4⁺ T cells from the first tumor or a metastatic tumor cell mass or an invasive tumor cell mass. In some embodiments, the cells are collected from the subject between day 4 and day 28 after illumination of the first tumor in the subject.

In some embodiments, the level, strength or extent of systemic immunity can be measured based on the number or percentage of intratumoral activated natural killer (NK) cells (e.g., CD49b⁺ CD3⁻Ki67⁺⁻ cells as a percentage of CD45⁺ cells, or CD49b⁺ CD3⁻CD69⁺ cells as a percentage of CD45⁺ cells). In some embodiments, the strength or extent of systemic immunity is measured by the number of intratumoral Ki-67⁺ NK cells and/or CD69⁺ NK cells or, such as CD49b⁺CD3⁻Ki-67⁺ NK cells and/or CD49b⁺CD3⁻CD69⁺ NK cells, and the systemic immunity against recurring tumors is increased or augmented if the percentage of intratumoral Ki-67⁺ NK cells (e.g., CD49b⁺CD3⁻Ki-67⁺ NK cells) and/or CD69⁺ NK cells (e.g., CD49b⁺CD3⁻CD69⁺ NK cells), among the total number of CD45⁺ cells, is increased after treatment as compared to before treatment. In some of such examples, the systemic immunity against recurring tumors is increased or augmented if the number of intratumoral Ki-67⁺ NK cells (e.g., CD49b⁺CD3⁻Ki-67⁺ NK cells) is at least at or about 0.03% of the total number of CD45⁺ cells, such as at least at or about 0.03%, 0.04%, 0.05%, 0.06%, 0.07%, 0.08% 0.09%, 0.10%, 0.11%, 0.12% 0.13%, 0.14%, 0.15%, 0.16%, 0.17%, 0.18%, 0.19%, 0.20%, or more of the total number of CD45⁺ cells. In some embodiments, the percentage of intratumoral Ki-67⁺ NK cells is at least 0.05% of the intratumoral CD45⁺ cell population. In some embodiments, the systemic immunity against recurring tumors is increased or augmented if the percentage of intratumoral Ki-67⁺ NK cells among the population of intratumoral CD45⁺ cells is increased after treatment as compared to before treatment. In some of such embodiments the percentage of intratumoral Ki-67⁺ NK cells among the population of intratumoral CD45⁺ cells is increased after treatment by at least at or about 0.02%, 0.03%, 0.04%, 0.05%, 0.06%, 0.07%, 0.08%, 0.09%, 0.10%, 0.11%, 0.12%, 0.13%, 0.14%, 0.15%, or more compared to before treatment. In some embodiments, the percentage of intratumoral Ki-67⁺ NK cells among the population of intratumoral CD45⁺ cells is increased after treatment by at least 5% as compared to before treatment.

In some of such examples, the systemic immunity against recurring tumors is increased or augmented if the number of intratumoral CD69⁺ NK cells (e.g., CD49b⁺CD3⁻CD69⁺ NK cells) is at least at or about 0.2% of the total number of CD45⁺ cells, such as at least at or about 0.2%, 0.25%, 0.3%, 0.35%, 0.4%, 0.45%, 0.5%, 0.55%, 0.6%, 0.65%, 0.7%, 0.75%, 0.8% 0.85%, 0.9%, 0.95%, 10%, or more of the total number of CD45⁺ cells. In some embodiments, the percentage of intratumoral CD69⁺ NK cells is at least 0.25% or at least 0.4% of the intratumoral CD45⁺ cell population. In some embodiments, the systemic immunity against recurring tumors is increased or augmented if the percentage of intratumoral CD69⁺ NK cells among the population of intratumoral CD45⁺ cells is increased after treatment as compared to before treatment. In some of such embodiments the percentage of intratumoral CD69⁺ NK cells among the population of intratumoral CD45⁺ cells is increased after treatment by at least at or about 0.1%, 0.15%, 0.2%, 0.25%, 0.3%, 0.35%, 0.4%, 0.45%, 0.5%, 0.55%, 0.6%, 0.65%, 0.7%, 0.75%, 0.8% 0.85%, 0.9%, 0.95%, 10%, or more compared to before treatment. In some embodiments, the percentage of intratumoral CD69⁺ NK cells among the population of intratumoral CD45⁺ cells is increased after treatment by at least 0.25% as compared to before treatment.

In some embodiments, any of the above assays can be used in combination. Typically, systemic immunity is assessed by assaying cells or components (e.g., cytokines) in circulation or located distal to the site or region of illumination. However, in some embodiments, systemic immunity is assessed by assaying cells or components (e.g., cytokines) within the illuminated tumor and/or the TME of the illuminated tumor. In some embodiments, the systemic immunity is assessed prior to treatment with any of the methods provided herein. In some embodiments, the systemic immunity is assessed after treatment with any of the provided methods. In some embodiments, the systemic immunity is assessed before and after treatment with any of the methods provided herein.

In some aspects, a systemic response may be assessed by assaying cells affected directly or indirectly by the methods. For example, samples can be collected from the subject between day 4 and day 28 after treatment or any time after the step of illumination of the first tumor in the subject. Samples can also be collected prior to conjugate administration to establish a baseline prior to treatment for comparison. In some embodiments, the strength or extent of systemic immunity is compared to the strength or extent of systemic immunity in the same subject prior to treatment. In some of such embodiments, the strength or extent of systemic immunity is compared to a population of subjects. In some of such embodiments, the strength or extent of systemic immunity is compared to a threshold value. In some embodiments, the strength or extent of systemic immunity following a combination therapy, such as anti-CTLA-4 PIT in combination with administration of a checkpoint inhibitor (e.g., anti-PD-1 antibody), is compared to the strength or extent of systemic immunity following treatment with a monotherapy, such as administration of a single agent, such as an immune checkpoint inhibitor (e.g., anti-PD-1 antibody) or anti-CTLA-4 conjugate or anti-CTLA-4 PIT alone.

In some embodiments, the methods and compositions herein provoke, stimulate, boost, augment, or support an immune response, such as a local response, such as a local immune response, in a subject having a cancer. In some embodiments, the method and uses herein includes enhancing a local response in a subject having a cancer, a tumor or a cancerous lesion. In some embodiments, the first tumor is a “cold” tumor that exhibits reduced immunoresponsiveness. “Local immune response” refers to the immune response in a tissue or an organ to an immunologic challenge or immunologic challenges including those associated with a cancer, a tumor, or a cancerous lesion. A local immune response can include the adaptive immune system and/or innate immune system. In some aspects, local immunity includes immune response concurrently occurring at different tissues, such as the blood stream, lymph node, bone marrow, spleen and/or the tumor microenvironment.

In some aspects, the provoked or increased local immune response includes an increase in the number and/or activity of intratumoral CD8⁺ T effector cells, an increase in CD8⁺ T effector cell activation, an increase in the intratumoral CD8⁺:Treg ratio, an increase in intratumoral natural killer cell infiltration, an increase in intratumoral natural killer cell activation, an increase in intratumoral dendritic cell activation, an increase in intratumoral dendritic cell infiltration, an increase in intratumoral new T cell priming, an increase in intratumoral T cell diversity, a decrease in intratumoral regulatory T cells, a decrease in intratumoral myeloid derived suppressor cells, a decrease in intratumoral tumor associated fibroblasts, a decrease in the number and/or activity of intratumoral exhausted PD-1⁺CTLA-4⁺CD3⁺CD8⁺ T cells, or any combination thereof in the subject.

In some aspects, the level, strength or extent of local immunity can be measured based on the number of intratumoral CD8⁺ T lymphocytes, the ratio of CD8⁺ T lymphocytes to regulatory T cells (Tregs), intratumoral T lymphocyte exhaustion (e.g., the percentage of CD3⁺CD8⁺ cells that express PD-1 and/or CTLA4 markers), the number or percentage of intratumoral activated CD8⁺ T lymphocytes (e.g., Ki67⁺ or CD69⁺ CD8 cells as a percentage of CD45⁺ cells), the expansion of cytotoxic intratumoral T lymphocytes (e.g., the percentage of CD3⁺CD8⁺ cells that do not express PD-1 and/or CTLA4 markers), based on the splenocyte cytotoxicity against tumor cells, or any or all of combination thereof. In some aspects, intratumoral CD8⁺ T lymphocytes include CD3⁺CD8⁺ cells, intratumoral exhausted T lymphocytes include PD-1⁺CTLA-4⁺CD3⁺CD8⁺ cells, activated intratumoral CD8⁺ T lymphocytes include CD3⁺CD8⁺Ki67⁺ and/or CD3⁺CD8⁺CD69⁺ cells, expansion of cytotoxic T lymphocytes include PD-1⁻CTLA-4⁻CD3⁺CD8⁺ cells. In some aspects, intratumoral CD8⁺ T lymphocytes, exhausted intratumoral T lymphocytes, activated CD8⁺ T lymphocytes, or expanded cytotoxic intratumoral T lymphocytes are measured as a percentage of leukocytes (CD45⁺ cells) and/or total CD8⁺ T cells (e.g., CD3⁺CD8⁺CD45⁺ cells). Determination of such numbers or percentages can be achieved using several well-known methods, including those described herein. For example, such numbers or percentages can be determined by generating single cell suspensions, such as by mechanical dissociation of tumor and/or tissue biopsies, or collection of blood samples containing circulating immune cells, followed by staining and flow cytometric analysis or mass cytometry. Other methods can include multiplexed immunofluorescence imaging of tissue and/or tumor biopsies.

In some embodiments, the strength or extent of local immunity is measured by the number of intratumoral CD8⁺ T lymphocytes, such as CD3⁺CD8⁺ T lymphocytes, and the local immunity against recurring tumors is increased or augmented if the percentage of intratumoral CD8⁺ T lymphocytes (e.g., CD3⁺CD8⁺ T lymphocytes), among the total number of CD45⁺ cells, is increased after treatment as compared to before treatment. In some of such examples, the local immunity against recurring tumors is increased or augmented if the number of intratumoral CD8⁺ T lymphocytes (e.g., CD3⁺CD8⁺ T lymphocytes) is at least at or about 30% of the total number of CD45⁺ cells, such as at least at or about 30%, 35%, 36%, 37%, 38% 39%, 40%, 41%, 42% 43%, 44%, 45%, 46%, 47%, 48%, 49%, 50%, 51%, 52%, 53%, 54%, 55%, 56%, 57%, 58%, 59%, 60%, or more of the total number of CD45⁺ cells. In some embodiments, the percentage of intratumoral CD8⁺ T lymphocytes is at least 40% of the intratumoral CD45⁺ cell population. In some embodiments, the local immunity against recurring tumors is increased or augmented if the percentage of intratumoral CD3⁺CD8⁺ T cells among the population of intratumoral CD45⁺ cells is increased after treatment as compared to before treatment. In some of such embodiments the percentage of intratumoral CD3⁺CD8⁺ T cells among the population of intratumoral CD45⁺ cells is increased after treatment by at least at or about 5%, 6%, 7%, 8%, 9%, 10%, 11%, 12%, 13%, 14%, 15%, 16%, 17%, 18%, 19%, 20%, 25%, 30%, or more compared to before treatment. In some embodiments, the percentage of intratumoral CD3⁺CD8⁺ T cells among the population of intratumoral CD45⁺ cells is increased after treatment by at least 10% as compared to before treatment.

In some embodiments, the strength or extent of local immunity is measured by the number of exhausted intratumoral CD8⁺ T lymphocytes, such as the number of PD-1⁺CTLA-4⁺CD3⁺CD8⁺ cells as a percentage of intratumoral CD8⁺ T lymphocytes (e.g., CD3⁺CD8⁺ T lymphocytes), and the local immunity against recurring tumors is increased or augmented if the percentage of PD-1⁺CTLA-4⁺CD3⁺CD8⁺ cells among intratumoral CD3⁺CD8⁺ T cells is decreased after treatment as comparted to before treatment. In some of such examples, the local immunity against recurring tumors is increased if the percentage of PD-1⁺CTLA-4⁺CD3⁺CD8⁺ cells among intratumoral CD3⁺CD8⁺ T cells after treatment is less than at or about 20%, such as less than 19%, 18%, 17%, 16%, 15%, 14%, 13%, 12%, 11%, 10%, 9%, 8%, 7%, 6%, 5%, 4%, 3%, 2%, 1%, or less. In some embodiments, the local immunity against recurring tumors is increased or augmented if the percentage of PD-1⁺CTLA-4⁺CD3⁺CD8⁺ cells among intratumoral CD3⁺CD8⁺ T cells is reduced after treatment compared to before treatment. In some of such embodiments, the percentage of PD-1⁺CTLA-4⁺CD3⁺CD8⁺ cells among intratumoral CD3⁺CD8⁺ T cells is decreased by at least at or about 5%, 6%, 7%, 8%, 9%, 10%, 11%, 12%, 13%, 14%, 15%, 16%, 17%, 18%, 19%, 20%, 25%, 30%, or more compared to before treatment. In some embodiments, the percentage of exhausted intratumoral CD8⁺ T cells (PD-1⁺CTLA-4⁺CD3⁺CD8⁺ cells) among the population of intratumoral CD3⁺CD8⁺ T cells is decreased after treatment by at least 10% as compared to before treatment.

In some embodiments, the strength or extent of local immunity is measured by the number of activated intratumoral CD8⁺ T lymphocytes, such as CD3⁺CD8⁺Ki67⁺ and/or CD3⁺CD8⁺CD69⁺ T lymphocytes, and the local immunity against recurring tumors is increased or augmented if the number of activated intratumoral CD8⁺ T lymphocytes, such as CD3⁺CD8⁺Ki67⁺ and/or CD3⁺CD8⁺CD69⁺ T lymphocytes, as a percentage of intratumoral CD45⁺ leukocytes, is increased after treatment as compared to before treatment. In some of such embodiments, the local immunity against recurring tumors is increased or augmented if the number of intratumoral CD3⁺CD8⁺Ki67⁺ cells, is at least at or about 0.15% of the total number of CD45⁺ cells, such as at least at or about 0.2%, 0.25%, 0.3%, 0.35%. 0.4%, 0.45%, 0.5%, or more of the total number of intratumoral CD45⁺ cells after treatment. In other of such embodiments, the local immunity against recurring tumors is increased or augmented if the number of intratumoral CD3⁺CD8⁺CD69⁺ cells, is at least at or about 0.5% of the total number of CD45⁺ cells, such as at least at or about 0.6%, 0.7%, 0.8%, 0.9%. 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, or more of the total number of intratumoral CD45⁺ cells, such as at least about 1.0% of the total number of intratumoral CD45⁺ cells after treatment. In some embodiments, the local immunity against recurring tumors is increased or augmented if the percentage of intratumoral CD3⁺CD8⁺Ki67⁺ and/or CD3⁺CD8⁺CD69⁺ T lymphocytes among intratumoral CD45⁺ cells is increased after treatment compared to before treatment. In some of such embodiments, the percentage of CD3⁺CD8⁺Ki67⁺ and/or CD3⁺CD8⁺CD69⁺ T lymphocytes cells among intratumoral CD45⁺ cells is increased by at least at or about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold-18-fold, 19-fold, 20-fold, 21-fold, 22-fold, 23-fold, 24-fold, 25-fold, 30-fold, 35-fold, 40-fold, 45-fold, 50-fold, 55-fold, 60-fold, or more compared to the percentage of CD3⁺CD8⁺Ki67⁺ and/or CD3⁺CD8⁺CD69⁺ T lymphocytes cells among intratumoral CD45⁺ cells before treatment. In some embodiments, the percentage of intratumoral CD3⁺CD8⁺Ki67⁺ T lymphocytes cells among intratumoral CD45⁺ cells is increased by at least 15-fold or 20-fold compared to the percentage of CD3⁺CD8⁺Ki67⁺ T lymphocytes cells among intratumoral CD45⁺ cells before treatment. In some embodiments, the percentage of intratumoral CD3⁺CD8⁺CD69⁺ T lymphocytes cells among intratumoral CD45⁺ cells is increased by at least 5-fold compared to the percentage of CD3⁺CD8⁺CD69⁺ T lymphocytes cells among intratumoral CD45⁺ cells before treatment.

In some embodiments, the strength or extent of local immunity is measured by the expansion of intratumoral cytotoxic T lymphocytes, such as PD-1⁻CTLA-4⁻CD3⁺CD8⁺ cells, and the local immunity against recurring tumors is increased or augmented if the percentage of intratumoral cytotoxic T lymphocytes (e.g., PD-1⁻CTLA-4⁻CD3⁺CD8⁺ cells) among CD8⁺ T cells (e.g., CD3⁺CD8⁺ T cells) is increased after treatment as compared to before treatment. In some of such examples, the local immunity against recurring tumors is increased or augmented if the number of intratumoral cytotoxic T lymphocytes (e.g., PD-1⁻CTLA-4⁻CD3⁺CD8⁺ cells) is at least at or about 20% of the total number of CD3⁺CD8⁺ T cells, such as at least at or about 25%, 30%, 35%, 40%, 41%, 42% 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, or more of the total number of CD45⁺ cells. In some embodiments, the percentage of intratumoral PD-1⁻CTLA-4⁻CD3⁺CD8⁺ cells is at least at or about 40%, 45%, 50%, or 55% of the intratumoral CD3⁺CD8⁺ T cell population. In some embodiments, the local immunity against recurring tumors is increased or augmented if the percentage of intratumoral PD-1⁻CTLA-4⁻CD3⁺CD8⁺ cells among the population of intratumoral CD3⁺CD8⁺ T cells is increased after treatment as compared to before treatment. In some of such embodiments the percentage of intratumoral PD-1⁻CTLA-4⁻CD3⁺CD8⁺ cells among the population of intratumoral CD3⁺CD8⁺ T cells is increased after treatment by at least at or about 5%, 10%, 15%, 20%, 25%, 30%, 35%, 40%, 50%, 60%, 70%, 75%, 80%, or more compared to the percentage of intratumoral PD-1⁻CTLA-4⁻CD3⁺CD8⁺ cells among the population of intratumoral CD3⁺CD8⁺ T cells before treatment. In some embodiments, the percentage of intratumoral PD-1⁻CTLA-4⁻CD3⁺CD8⁺ cells among the population of intratumoral CD3⁺CD8⁺ T cells is increased after treatment by at least 30% as compared to the percentage of intratumoral PD-1⁻CTLA-4⁻CD3⁺CD8⁺ cells among the population of intratumoral CD3⁺CD8⁺ T cells before treatment.

In some embodiments, treatment in accordance with the methods and uses provided herein, leads to cell death of or a reduction in number of regulatory T cells (Tregs), such as intratumoral CD4⁺FoxP3⁺ Tregs, which can contribute to local immunity. Hence, in some embodiments, the level, strength, or extent of local immunity can be measured based on the number or percentage of intratumoral Tregs. In some aspects, binding of the anti-CTLA-4 conjugate to the surface of CTLA-4-expressing cells, such as certain Tregs, and illumination to effect illumination-dependent lysis and death of intratumoral cells expressing CTLA-4, results in a reduction of the number of cells expressing CTLA-4. In some aspects, such results lead to a reduction in the number of immunosuppressive cells, such as Tregs, within the tumor, and thus can alleviate or reverse immunosuppression in the tumor. In some aspects, such reduction in immunosuppressive cells can result in the activation and proliferation of intratumoral T cells, such as intratumoral CD8⁺ cytotoxic T cells or CD4⁺ helper T cells, that can eliminate tumor cells, and lead to reduction of tumor volume and/or elimination of the tumor. In some aspects, treatment in accordance with the provided embodiments can result in a reduction of intratumoral Tregs and/or an increase of intratumoral CD8⁺ to Treg ratio or intratumoral CD4⁺ to Treg ratio. In some embodiments of the provided methods and uses, systemic Tregs are not reduced as a result of treatment.

In some aspects, treatment in accordance with the methods and uses provided herein can result in a lasting or durable decrease in intratumoral Tregs. In some aspects, treatment in accordance with the methods and uses provided herein can result in a lasting or durable increase of intratumoral CD8⁺ to Treg ratio or intratumoral CD4⁺ to Treg ratio. In some embodiments, the level, strength or extent of local immunity can be measured by determining the intratumoral CD8⁺ to Treg ratio, and the local immunity against recurring tumors is increased or augmented if the intratumoral CD8⁺ to Treg ratio is increased after treatment as compared to before treatment. In some of such examples, the local immunity against recurring tumors is increased or augmented if the intratumoral CD8⁺ to Treg ratio is increased by at least at or about 1-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold. 4.0-fold, or more compared to the intratumoral CD8⁺ to Treg ratio before treatment. In some embodiments, the level, strength or extent of local immunity can be measured by determining the intratumoral CD4⁺ to Treg ratio, and the local immunity against recurring tumors is increased or augmented if the intratumoral CD4⁺ to Treg ratio is increased after treatment as compared to before treatment. In some embodiments, the level, strength or extent of local immunity can be measured by determining the intratumoral Treg to CD45⁺ ratio, and the local immunity against recurring tumors is increased or augmented if the intratumoral Treg to CD45⁺ ratio is decreased after treatment as compared to before treatment. In some aspects, such increases or decreases can last for at or about 3, 4, 5, 6, 7, 8, 9, 10, 11, 12, 13, 14 days or 3, 4, 5, 6, 7 or 8 weeks or longer.

In some aspects, the level, strength or extent of local immunity can be measured by a CTL activity assay using splenocytes or peripheral blood cells or bone marrow cells or lymph node cells. In some embodiments, the cells are collected from the subject between day 4 and day 28 after illumination of the first tumor in the subject.

In some aspects, the level, strength or extent of local immunity can be measured by an intratumoral T cell exhaustion assay using T cells collected from the first tumor or a metastatic tumor cells mass or an invasive tumor cell mass. In some embodiments, the cells are collected from the subject between day 4 and day 28 after illumination of the first tumor in the subject.

In some aspects, the level, strength or extent of local immunity can be measured by an intratumoral effector T cell expansion assay using T cells collected from the first tumor or a metastatic tumor cells mass or an invasive tumor cell mass. In some embodiments, the cells are collected from the subject between day 4 and day 28 after illumination of the first tumor in the subject.

In some aspects, the level, strength or extent of local immunity can be measured by a T cell receptor diversity assay using T cells collected from the first tumor or a metastatic tumor cells mass or an invasive tumor cell mass or the peripheral circulation. In some embodiments, the cells are collected from the subject between day 4 and day 28 after illumination of the first tumor in the subject.

In some aspects, the level, strength or extent of local immunity can be measured by determining the presence, number or frequency of regulatory T cells (Tregs) in the tumor and/or the ratio of intratumoral Treg cells to intratumoral CD8⁺ T cells or intratumoral CD4⁺ T cells from the first tumor or a metastatic tumor cell mass or an invasive tumor cell mass. In some embodiments, the cells are collected from the subject between day 4 and day 28 after illumination of the first tumor in the subject.

In some embodiments, any of the above assays can be used in combination. Typically, local immunity is assessed by assaying cells or components (e.g., cytokines) within the illuminated tumor and/or the TME of the illuminated tumor. However, in some embodiments, local immunity is assessed by assaying cells or components (e.g., cytokines) in circulation or located distal to the site or region of illumination.

In some embodiments, the level, strength or extent of local immunity can be measured based on the number or percentage of intratumoral activated natural killer (NK) cells (e.g., CD49b⁺CD3⁻Ki67⁺⁻ cells as a percentage of CD45⁺ cells, or CD49b⁺CD3⁻CD69⁺ cells as a percentage of CD45⁺ cells). In some embodiments, the strength or extent of local immunity is measured by the number of intratumoral Ki-67⁺ NK cells and/or CD69⁺ NK cells or, such as CD49b⁺CD3⁻Ki-67⁺ NK cells and/or CD49b⁺CD3⁻CD69⁺ NK cells, and the local immunity against recurring tumors is increased or augmented if the percentage of intratumoral Ki-67⁺ NK cells (e.g., CD49b⁺CD3⁻Ki-67⁺ NK cells) and/or CD69⁺ NK cells (e.g., CD49b⁺CD3⁻CD69⁺ NK cells), among the total number of CD45⁺ cells, is increased after treatment as compared to before treatment. In some of such examples, the local immunity against recurring tumors is increased or augmented if the number of intratumoral Ki-67⁺ NK cells (e.g., CD49b⁺CD3⁻Ki-67⁺ NK cells) is at least at or about 0.03% of the total number of CD45⁺ cells, such as at least at or about 0.03%, 0.04%, 0.05%, 0.06%, 0.07%, 0.08% 0.09%, 0.10%, 0.11%, 0.12% 0.13%, 0.14%, 0.15%, 0.16%, 0.17%, 0.18%, 0.19%, 0.20%, or more of the total number of CD45⁺ cells. In some embodiments, the percentage of intratumoral Ki-67⁺ NK cells is at least 0.05% of the intratumoral CD45⁺ cell population. In some embodiments, the local immunity against recurring tumors is increased or augmented if the percentage of intratumoral Ki-67⁺ NK cells among the population of intratumoral CD45⁺ cells is increased after treatment as compared to before treatment. In some of such embodiments the percentage of intratumoral Ki-67⁺ NK cells among the population of intratumoral CD45⁺ cells is increased after treatment by at least at or about 0.02%, 0.03%, 0.04%, 0.05%, 0.06%, 0.07%, 0.08%, 0.09%, 0.10%, 0.11%, 0.12%, 0.13%, 0.14%, 0.15%, or more compared to before treatment. In some embodiments, the percentage of intratumoral Ki-67⁺ NK cells among the population of intratumoral CD45⁺ cells is increased after treatment by at least 5% as compared to before treatment.

In some of such examples, the local immunity against recurring tumors is increased or augmented if the number of intratumoral CD69⁺ NK cells (e.g., CD49b⁺CD3⁻CD69⁺ NK cells) is at least at or about 0.2% of the total number of CD45⁺ cells, such as at least at or about 0.2%, 0.25%, 0.3%, 0.35%, 0.4%, 0.45%, 0.5%, 0.55%, 0.6%, 0.65%, 0.7%, 0.75%, 0.8% 0.85%, 0.9%, 0.95%, 10%, or more of the total number of CD45⁺ cells. In some embodiments, the percentage of intratumoral CD69⁺ NK cells is at least 0.25% or at least 0.4% of the intratumoral CD45⁺ cell population. In some embodiments, the local immunity against recurring tumors is increased or augmented if the percentage of intratumoral CD69⁺ NK cells among the population of intratumoral CD45⁺ cells is increased after treatment as compared to before treatment. In some of such embodiments the percentage of intratumoral CD69⁺ NK cells among the population of intratumoral CD45⁺ cells is increased after treatment by at least at or about 0.1%, 0.15%, 0.2%, 0.25%, 0.3%, 0.35%, 0.4%, 0.45%, 0.5%, 0.55%, 0.6%, 0.65%, 0.7%, 0.75%, 0.8% 0.85%, 0.9%, 0.95%, 10%, or more compared to before treatment. In some embodiments, the percentage of intratumoral CD69⁺ NK cells among the population of intratumoral CD45⁺ cells is increased after treatment by at least 0.25% as compared to before treatment. In some cases, a local response, such as a local immune response, can be assessed by taking a blood, tissue or other sample from a subject and assessing for an increase in an anti-immune cell type in the tumor or TME and/or assessing for an increase or appearance of local immune activation markers.

In some embodiments, the systemic immunity is assessed prior to treatment with any of the methods provided herein. In some embodiments, the systemic immunity is assessed after treatment with any of the provided methods. In some embodiments, the systemic immunity is assessed before and after treatment with any of the methods provided herein.

In some aspects, a local response, such as a local immune response, may be assessed by assaying cells affected directly or indirectly by the methods. For example, cell can be collected from the subject between day 4 and day 28 after treatment or any time after the step of illumination of the first tumor in the subject.

### III. COMPOSITIONS FOR USE WITH THE METHODS, INCLUDING MONOTHERAPY AND COMBINATION THERAPY METHODS

The methods and uses provided herein employ an anti-CTLA-4 conjugate that includes a targeting molecule that binds to CTLA-4 linked to a phthalocyanine dye. Cytotoxic T-lymphocyte-associated protein 4 (CTLA-4 or CTLA4), also known as cluster of differentiation 152 (CD152)), is a protein receptor that functions as an immune checkpoint and downregulates immune responses. CTLA-4 is constitutively expressed in CD4⁺FoxP3⁺ regulatory T cells (Tregs), in activated T cells, and in some tumor cells. It acts as an “off” switch when bound to CD80 or CD86 on the surface of antigen-presenting cells.

In some embodiments, the targeting molecule can be an anti-CTLA-4 antibody or antigen-binding fragment thereof. Exemplary anti-CTLA-4 antibodies include, but are not limited to, are ipilimumab (YERVOY), tremelimumab (ticilimumab, CP-675,206), AGEN1181, AGEN1884, ADU-1064, BCD-145, BCD-217, ADG116, AK104, ATOR-1015, BMS-986218, KN046, MGD019, MK-1308, REGN4659, XmAb20717, and XmAb22841.

In some embodiments, the targeting molecule can be an antibody or antibody fragment that includes the “complementarity-determining regions” or “CDRs” of an anti-CTLA-4 antibody, such as any of the described antibodies or antigen-binding fragment thereof. The CDRs are typically responsible for binding to an epitope of an antigen. The CDRs of each chain are typically referred to as CDR1, CDR2, and CDR3, numbered sequentially starting from the N-terminus, and are also generally identified by the chain in which the particular CDR is located. Thus, a heavy chain variable region (V_{H}) CDR3 is located in the variable domain of the heavy chain of the antibody in which it is found, whereas a light chain variable region (V_{L}) CDR1 is the CDR1 from the variable domain of the light chain of the antibody in which it is found. Antibodies with different specificities, such as different combining sites for different antigens, have different CDRs. Although it is the CDRs that vary from antibody to antibody, only a limited number of amino acid positions within the CDRs are directly involved in antigen binding. These positions within the CDRs are called specificity determining residues (SDRs). In some embodiments, the targeting molecule includes CDRs from ipilimumab (YERVOY), tremelimumab (ticilimumab), AGEN1181, AGEN1884, ADU-1064, BCD-145, or BCD-217.

In some embodiments, the targeting molecule of an anti-CTLA-4 conjugate is ipilimumab (YERVOY) or tremelimumab (ticilimumab). In some embodiments, the targeting molecule of an anti-CTLA-4 conjugate is a biosimilar, interchangeable or biobetter of any of the anti-CTLA-4 antibody described herein, e.g., ipilimumab (YERVOY) or tremelimumab (ticilimumab), or an antigen-binding fragment thereof. Such antibodies also include copy biologicals and biogenerics of any of the anti-CTLA-4 antibodies described herein, e.g., ipilimumab (YERVOY), tremelimumab (ticilimumab), or an antigen-binding fragment thereof.

In some embodiments, the targeting molecule of an anti-CTLA-4 antibody comprises a functional Fc region. In some embodiments, the targeting molecule of an anti-CTLA-4 antibody is a humanized antibody.

The anti-CTLA-4 conjugates used in the methods and compositions herein include a phthalocyanine dye. In some embodiments, the phthalocyanine dye is a phthalocyanine dye with a silicon coordinating metal (Si-phthalocyanine dye). In some embodiments, the phthalocyanine dye comprises the formula:

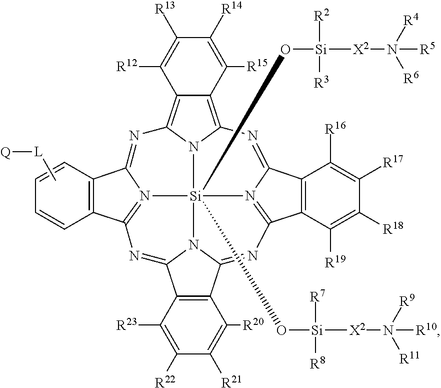

wherein:

L is a linker;

Q is a reactive group for attachment of the dye to the targeting molecule;

R², R³, R⁷, and R⁸ are each independently selected from among optionally substituted alkyl and optionally substituted aryl;

R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group;

R¹², R¹³, R¹⁴, R¹⁵, R¹⁶, R¹⁷, R¹⁸, R¹⁹, R²⁰, R²¹, R²² and R²³ are each independently selected from among hydrogen, halogen, optionally substituted alkylthio, optionally substituted alkylamino and optionally substituted alkoxy; and

X² and X³ are each independently C₁-C₁₀ alkylene, optionally interrupted by a heteroatom.

In some embodiments, the phthalocyanine dye comprises the formula:

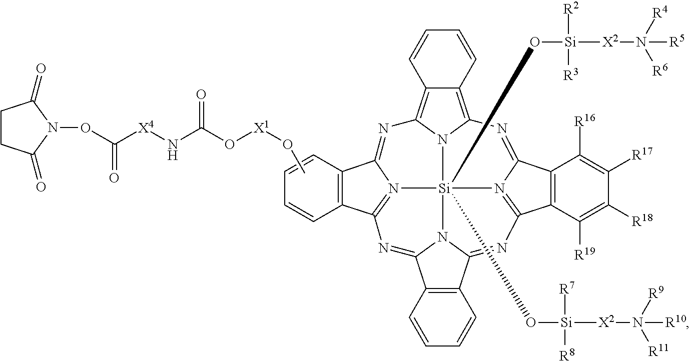

wherein:

X¹ and X⁴ are each independently a C₁-C₁₀ alkylene optionally interrupted by a heteroatom;

R², R³, R⁷, and R⁸ are each independently selected from optionally substituted alkyl and optionally substituted aryl;

R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group; and

R¹⁶, R¹⁷, R¹⁸ and R¹⁹ are each independently selected from among hydrogen, halogen, optionally substituted alkylthio, optionally substituted alkylamino and optionally substituted alkoxy.

In some embodiments of the methods and uses provided herein, a Si-phthalocyanine dye is IRDye 700DX (IR700). In some embodiments, the phthalocyanine dye containing the reactive group is IR700 NHS ester, such as IRDye 700DX NHS ester (LiCor 929-70010, 929-70011). In some embodiments, the dye is a compound having the following formula:

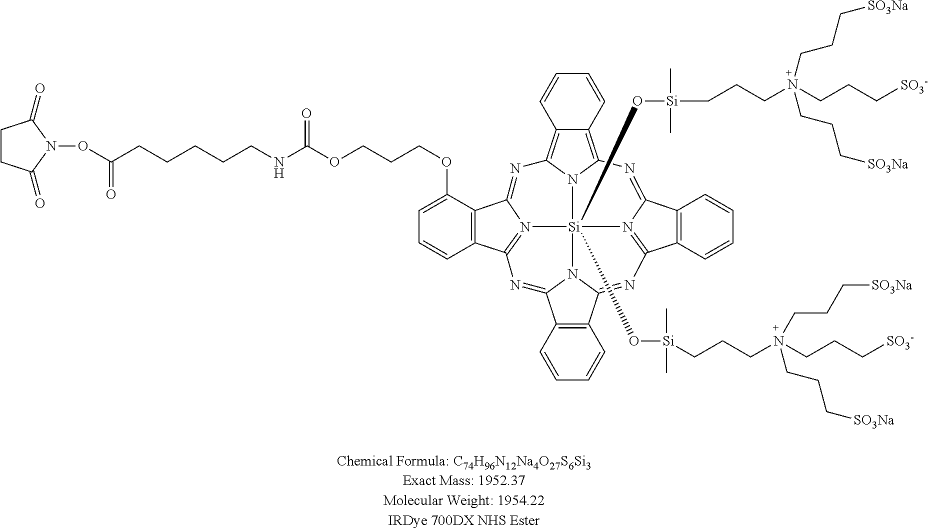

For purposes herein, the term “IR700,” “IRDye 700” or “IRDye 700DX” includes the above formula when the dye is conjugated such as to an antibody, e.g. via a reactive group.

In some embodiments the compositions for use with the methods herein include an anti-CTLA-4 conjugate comprising a Si-phthalocyanine dye linked to targeting molecule that binds to CTLA-4. In some embodiments, the composition is an anti-CTLA-4-Si-phthalocyanine dye conjugate. In some embodiments, the composition is an anti-CTLA-4-IR700 conjugate. In some embodiments, the composition is an anti-CTLA-4-IR700 conjugate, where the targeting molecule is ipilimumab or tremelimumab. In some embodiments, the composition is an anti-CTLA-4-IR700 conjugate, where the targeting molecule is ipilimumab containing a functional Fc region, or is tremelimumab containing a functional Fc region.

### IV. COMBINATION THERAPY

In some embodiments, the methods herein include combination treatments that include an anti-CTLA-4 conjugate in combination with an immune modulatory agent. In some embodiments, the targeting molecule used in such combination treatments is an anti-CTLA-4 antibody, or an antibody fragment that binds to CTLA-4. In some embodiments, the conjugate is an anti-CTLA-4 antibody, or an antibody fragment that binds to CTLA-4 linked to a Si-phthalocyanine dye, such as an IR700 dye.

The immune modulatory agent used in such combination treatments herein can include an adjuvant, immune checkpoint inhibitor, cytokine or any combination thereof. A cytokine for use in the combinations can be, for example, Aldesleukin (PROLEUKIN), Interferon alfa-2a, Interferon alfa-2b (Intron A), Peginterferon Alfa-2b (SYLATRON/PEG-Intron), or a cytokine that targets the IFNAR1/2 pathway, the IL-2/IL-2R pathway. An adjuvant for use in the combinations can be, for example, Poly ICLC (HILTONOL/Imiquimod), 4-1BB (CD137; TNFRS9), OX40 (CD134) OX40-Ligand (OX40L), Toll-Like Receptor 2 Agonist SUP3, Toll-Like Receptor TLR3 and TLR4 agonists and adjuvants targeting the Toll-like receptor 7 (TLR7) pathway, other members of the TNFR and TNF superfamilies, other TLR2 agonists, TLR3 agonists and TLR4 agonists.

For the combination therapies herein, the immune checkpoint inhibitor can be a PD-1 inhibitor, such as a small molecule, antibody or antigen binding fragment. In some aspects, the immune checkpoint inhibitor is an anti-PD-1 antibody or an antigen-binding fragment thereof. Exemplary anti-PD-1 antibodies include, but are not limited to pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, TSR-042 (ANB011), and any combination thereof. In some embodiments, the immune checkpoint inhibitor is pembrolizumab (MK-3475, KEYTRUDA; lambrolizumab), nivolumab (OPDIVO) or cemiplimab (LIBTAYO), or an antigen-binding fragment thereof. In some embodiments, the immune checkpoint inhibitor is a biosimilar, interchangeable, biobetter, copy biologicals or biogenerics of any of the anti-PD-1 antibody described herein, e.g., pembrolizumab (MK-3475, KEYTRUDA; lambrolizumab), nivolumab (OPDIVO) or cemiplimab (LIBTAYO), or an antigen-binding fragment thereof.

For the combination therapies herein, the immune checkpoint inhibitor can be a PD-L1 inhibitor, such as a small molecule, antibody or antigen binding fragment. In some aspects, the immune checkpoint inhibitor is an anti-PD-L1 antibody or an antigen-binding fragment thereof. Exemplary anti-PD-L1 antibodies include, but are not limited to, atezolizumab (MPDL3280A, TECENTRIQ, RG7446), avelumab (BAVENCIO, MSB0010718C; M7824), durvalumab (MEDI4736, IMFINZI), LDP, NM-01, STI-3031 (IMC-001; STI-A1015), KN035, LY3300054, M7824 (MSB0011359C), BMS-936559, MSB2311, BCD-135, BGB-A333, CBT-502 (TQB-2450), cosibelimab (CK-301), CS1001 (WPB3155), FAZ053, MDX-1105, SHR-1316 (HTI-1088), TG-1501, ZKAB001 (STI-A1014), INBRX-105, MCLA-145, KN046, LY3415244, REGN3504, HLX20, and any combination thereof. In some embodiments, the immune checkpoint inhibitor is atezolizumab (MPDL3280A, TECENTRIQ, RG7446), avelumab (BAVENCIO, MSB0010718C; M7824), durvalumab (MEDI4736, IMFINZI), or an antigen-binding fragment thereof. In some embodiments, the immune checkpoint inhibitor is a biosimilar, interchangeable, biobetter, copy biologicals or biogenerics of any of the anti-PD-L1 antibody described herein, e.g., atezolizumab (MPDL3280A, TECENTRIQ, RG7446), avelumab (BAVENCIO, MSB0010718C; M7824), durvalumab (MEDI4736, IMFINZI), or an antigen-binding fragment thereof.

The administration of an immune modulatory agent, such as a checkpoint inhibitor, adjuvant or cytokine, can be administered prior to, concurrent with, or subsequent to the administration of the anti-CTLA-4 conjugate. For example, the methods can include administering one or more doses of an immune checkpoint inhibitor, administering an anti-CTLA-4 conjugate, and after administration of the conjugate, illuminating with a suitable wavelength of light one or more first tumors. The methods can include first administering the conjugate, and after administration of the conjugate, illuminating one or more first tumors, and then administering an immune modulatory agent, such as an immune checkpoint inhibitor, subsequently either to administration of the conjugate or subsequently to the illumination step. The methods can also include the administration of an immune modulatory agent, such as an immune checkpoint inhibitor, concurrently with administration of the conjugate, followed by illuminating one or more first tumors. In some embodiments, an immune modulatory agent, such as an immune checkpoint inhibitor, adjuvant or cytokine, is administered one or more times, prior to when an anti-CTLA-4 conjugate is administered, followed by illuminating one or more first tumors, and then one or more additional administrations of an immune modulatory agent (the same or a different an immune modulatory agent).

In some embodiments, the immune modulatory agent is an immune checkpoint inhibitor such as a PD-1 inhibitor, a PD-L1 inhibitor, or any other or a combination thereof. In some embodiments, the immune checkpoint inhibitor is selected from an antibody or antigen-binding fragment that binds to and inhibits PD-1 or PD-L1. In some embodiments, the immune checkpoint inhibitor is a small molecule that inhibits PD-1 or PD-L1, or a peptide that blocks the binding of PD-L1 to PD-1.

In some embodiments, the combination therapies herein include administration of the immune checkpoint inhibitor prior to the administration of an anti-CTLA-4 conjugate and illumination. In some aspects, the immune checkpoint inhibitor can be administered to a subject one week, two weeks, three weeks, four weeks, or more than four weeks prior to the administration of the conjugate. In some aspects, the immune checkpoint inhibitor can be administered to the subject one time, twice, three times, four times, five times, or more than five times prior to the administration of the conjugate.

In some embodiments, the methods include the administration of an immune checkpoint inhibitor concurrent with the administration of an anti-CTLA-4 conjugate, and subsequently a first tumor is illuminated. In some aspects, following the illumination of the tumor, the immune checkpoint inhibitor can be further administered to the subject one time, twice, three times, four times, five times, or more than five times.

In some embodiments, the methods include the administration of the immune checkpoint inhibitor subsequent to the administration of an anti-CTLA-4 conjugate. In some aspects, the immune checkpoint inhibitor is administered to a subject having cancer one day after administering the conjugate, within one week after administering the conjugate, within two weeks after administering the conjugate, within three weeks after administering the conjugate, or within four week after administering the conjugate. In some aspects, the immune checkpoint inhibitor can be administered to a subject one time, twice, three times, or more than three times.

In some embodiments the methods include further administering an additional therapeutic agent or anti-cancer treatment.

### V. METHODS OF ADMINISTRATION AND FORMULATIONS

In some embodiments, the anti-CTLA-4 conjugate may be administered, for example, to a subject that has a disease or condition such as a cancer or a tumor, or a lesion, either systemically or locally to the organ or tissue to be treated. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes. In some embodiments, the anti-CTLA-4 conjugate is administered intravenously. In some embodiments, the anti-CTLA-4 conjugate is administered parenterally. In some embodiments, the anti-CTLA-4 conjugate is administered enterally. In some embodiments, the conjugate is administered by local injection. In some embodiments, the conjugate is administered as a topical application.

The compositions comprising the anti-CTLA-4 conjugate can be administered locally or systemically using any method known in the art, for example to subjects having a tumor, such as a cancer, or who has had a tumor previously removed, for example via surgery. Although specific examples are provided, one skilled in the art will appreciate that alternative methods of administration of the disclosed agents can be used. Such methods may include for example, the use of catheters or implantable pumps to provide continuous infusion over a period of several hours to several days into the subject in need of treatment.

In some embodiments, the anti-CTLA-4 conjugate is administered by parenteral means, including direct injection or infusion into a tumor, such as intratumorally. In some embodiments, the anti-CTLA-4 conjugate is administered to the tumor by applying the agent to the tumor, for example by bathing the tumor in a solution containing the anti-CTLA-4 conjugate, or by pouring the agent onto the tumor.

In addition, or alternatively, the anti-CTLA-4 conjugate can be administered systemically, for example intravenously, intramuscularly, subcutaneously, intradermally, intraperitoneally, subcutaneously, or orally, to a subject having a tumor, such as cancer.

The dosages of the anti-CTLA-4 conjugate to be administered to a subject are not subject to absolute limits, but will depend on the nature of the composition and its active ingredients and its unwanted side effects, such as immune response against the agent, the subject being treated, and the type of condition being treated and the manner of administration. Generally, the dose will be a therapeutically effective amount, such as an amount sufficient to achieve a desired biological effect, for example an amount that is effective to decrease the size, such as volume and/or weight, of the tumor, or attenuate further growth of the tumor, or decrease undesired symptoms of the tumor.

In some embodiments, the compositions used for administration of the anti-CTLA-4 conjugate contain an effective amount of the agent along with conventional pharmaceutical carriers and excipients appropriate for the type of administration contemplated. For example, in some embodiments, parenteral formulations may contain a sterile aqueous solution or suspension of the conjugate. In some embodiments, compositions for enteral administration may contain an effective amount of the anti-CTLA-4 conjugate in aqueous solution or suspension that may optionally include buffers, surfactants, thixotropic agents, and flavoring agents.

In some embodiments, the anti-CTLA-4 conjugate or conjugate in combination with another therapeutic agent, can be formulated in a pharmaceutically acceptable buffer, such as that containing a pharmaceutically acceptable carrier or vehicle. Generally, the pharmaceutically acceptable carriers or vehicles, such as those present in the pharmaceutically acceptable buffer, are can be any known in the art. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds. Pharmaceutically acceptable compositions generally are prepared in view of approvals for a regulatory agency or other agency prepared in accordance with generally recognized pharmacopeia for use in animals and in humans.

Pharmaceutical compositions can include carriers such as a diluent, adjuvant, excipient, or vehicle with which the compound is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidone, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

In some embodiments, pharmaceutical preparation can be in liquid form, for example, solutions, syrups or suspensions. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In some cases, pharmaceutical preparations can be presented in lyophilized form for reconstitution with water or other suitable vehicle before use.

In some embodiments, the nature of the pharmaceutically acceptable buffer, or carrier, depends on the particular mode of administration being employed. For instance, in some embodiments, parenteral formulations may comprise injectable fluids that include pharmaceutically and physiologically acceptable fluids such as water, physiological saline, balanced salt solutions, aqueous dextrose, or glycerol as a vehicle. In some embodiments, for solid compositions, for example powder, pill, tablet, or capsule forms, non-toxic solid carriers can include, for example, pharmaceutical grades of mannitol, lactose, starch, or magnesium stearate. In addition to biologically-neutral carriers, pharmaceutical compositions to be administered can in some embodiments contain minor amounts of non-toxic auxiliary substances, such as wetting or emulsifying agents, preservatives, and pH buffering agents, for example sodium acetate or sorbitan monolaurate.

The compounds can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compounds are formulated into pharmaceutical compositions using techniques and procedures well known in the art (see e.g., Ansel Introduction to Pharmaceutical Dosage Forms, Fourth Edition, 1985, 126). Generally, the mode of formulation is a function of the route of administration.

Compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. Other modes of administration also are contemplated. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant.

Parenteral administration, generally characterized by injection, either subcutaneously, intramuscularly, intratumorally, intravenously or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain an activator in the form of a solvent such as pH buffering agents, metal ion salts, or other such buffers. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) also is contemplated herein. The percentage of active compound contained in such parenteral compositions is highly dependent on the specific nature thereof, as well as the activity of the compound and the needs of the subject.

Injectables are designed for local and systemic administration. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or non-aqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, non-aqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Non-aqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate.

If administered intravenously, suitable carriers include physiological saline or phosphate buffered saline (PBS), and solutions containing thickening and solubilizing agents, such as glucose, polyethylene glycol, and polypropylene glycol and mixtures thereof.

The composition can be formulated for single dosage administration or for multiple dosage administration. The agents can be formulated for direct administration. The composition can be provided as a liquid or lyophilized formulation. Where the composition is provided in lyophilized form it can be reconstituted just prior to use by an appropriate buffer, for example, a sterile saline solution.

Compositions also can be administered with other biologically active agents, either sequentially, intermittently or in the same composition. Administration also can include controlled release systems including controlled release formulations and device-controlled release, such as by means of a pump.

The most suitable route in any given case depends on a variety of factors, such as the nature of the disease, the progress of the disease, the severity of the disease and the particular composition which is used. For example, compositions are administered systemically, for example, via intravenous administration. Subcutaneous methods also can be employed, although increased absorption times can be necessary to ensure equivalent bioavailability compared to intravenous methods.

Pharmaceutical compositions can be formulated in dosage forms appropriate for each route of administration. Pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Unit dosage forms, include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared. Pharmaceutical compositions can be formulated in dosage forms appropriate for each route of administration.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. All preparations for parenteral administration must be sterile, as is known and practiced in the art. In some embodiments, the compositions can be provided as a lyophilized powder, which can be reconstituted for administration as solutions, emulsions and other mixtures. They may also be reconstituted and formulated as solids or gels. The lyophilized powders can be prepared from any of the solutions described above.

The sterile, lyophilized powder can be prepared by dissolving a phthalocyanine dye-targeting molecule conjugate in a buffer solution. The buffer solution may contain an excipient which improves the stability of other pharmacological components of the powder or reconstituted solution, prepared from the powder.

In some embodiments, subsequent sterile filtration of the solution followed by lyophilization under standard conditions known to those of skill in the art provides the desired formulation. Briefly, the lyophilized powder is prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to ensure sterility, and apportioned into vials for lyophilization. Each vial can contain a single dosage (1 mg-1 g, generally 1-100 mg, such as 1-5 mg) or multiple dosages of the compound. The lyophilized powder can be stored under appropriate conditions, such as at about 4 .degree. C. to room temperature. Reconstitution of this lyophilized powder with a buffer solution provides a formulation for use in parenteral administration. The precise amount depends upon the indication treated and selected compound. Such amount can be empirically determined.

In some embodiments, the pH of the composition is between or between about 6 and 10, such as between or between about 6 and 8, between or between about 6.9 and 7.3, such as about pH 7.1. In some embodiments, the pH of the pharmaceutically acceptable buffer is at least or about 5, at least or about 6, at least or about 7, at least or about 8, at least or about 9 or at least or about 10, or is 7.1.

The compositions can be formulated for single dosage administration or for multiple dosage administration. The agents can be formulated for direct administration.

In some embodiments, the compositions provided herein are formulated in an amount for direct administration of the anti-CTLA-4 conjugate, in a range from or from about 0.01 mg to about 3000 mg, from about 0.01 mg to about 1000 mg, from about 0.01 mg to about 500 mg, from about 0.01 mg to about 100 mg, from about 0.01 mg to about 50 mg, from about 0.01 mg to about 10 mg, from about 0.01 mg to about 1 mg, from about 0.01 mg to about 0.1 mg, from about 0.1 mg to about 2000 mg, from about 0.1 mg to about 1000 mg, from about 0.1 mg to about 500 mg, from about 0.1 mg to about 100 mg, from about 0.1 mg to about 50 mg, from about 0.1 mg to about 10 mg, from about 0.1 mg to about 1 mg, from about 1 mg to about 2000 mg, from about 1 mg to about 1000 mg, from about 1 mg to about 500 mg, from about 1 mg to about 100 mg, from about 1 mg to about 10 mg, from about 10 mg to about 2000 mg, from about 10 mg to about 1000 mg, from about 10 mg to about 500 mg, from about 10 mg to about 100 mg, from about 100 mg to about 2000 mg, from about 100 mg to about 1000 mg, from about 100 mg to about 500 mg, from about 500 mg to about 2000 mg, from about 500 mg to about 1000 mg, and from about 1000 mg to about 3000 mg. In some embodiments, the volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL or more. For example, the composition is formulated for single dosage administration of an amount between or between about 100 mg and 500 mg, or between or between about 200 mg and 400 mg. In some embodiments, the composition is formulated for single dosage administration of an amount between or between about 500 mg and 1500 mg, 800 mg and 1200 mg or 1000 mg and 1500 mg. In some embodiments, the volume of the composition is between or between about 10 mL and 1000 mL or 50 mL and 500 mL; or the volume of the composition is at least 10 mL, 20 mL, 30 mL, 40 mL, 50 mL, 75 mL, 100 mL, 150 mL, 200 mL, 250 mL, 300 mL, 400 mL, 500 mL or 1000 mL.

In some embodiments, the entire vial contents of the formulations can be withdrawn for administration, or can be divided up into a plurality of dosages for multiple administrations. Upon withdrawal of an amount of drug for administration, the formulation can be further diluted if desired, such as diluted in water, saline (e.g., 0.9%) or other physiological solution.

In some embodiments, also provided are compositions containing an immune modulating agent or anti-cancer agent, which can be prepared in accord with known or standard formulation guidelines, such as described above. In some embodiments, the immune modulating agent, anti-cancer agent and/or anti-CTLA-4 conjugate are formulated as separate compositions. In some embodiments, the immune modulating agent is provided as a separate composition from the anti-CTLA-4 conjugate, and the two compositions are administered separately. In some embodiments, the anti-cancer agent is provided as a separate composition from the anti-CTLA-4 conjugate, and the two compositions are administered separately. The compositions can be formulated for parenteral delivery (i.e. for systemic delivery). For example, the compositions or combination of compositions are formulated for subcutaneous delivery or for intravenous delivery. The agents, such as an anti-CTLA-4 conjugate, and an immune modulating agent, and/or an anti-cancer agent can be administered by different routes of administration.

The following are exemplary administrations of immune modulatory agents and such agents can be administered as such or on other administration schedules and doses. For example, PD-1 inhibitor pembrolizumab (KEYTRUDA), the recommended dose for melanoma patients is 2 mg/kg administered as an intravenous infusion over 30 minutes every 3 weeks until disease progression or unacceptable toxicity. For non-small cell lung cancer, the recommended dose of KEYTRUDA is 200 mg administered as an intravenous infusion over 30 minutes every 3 weeks until disease progression, unacceptable toxicity, or up to 24 months in patients without disease progression. For patients with head and neck squamous cell carcinoma, the recommended dose of KEYTRUDA is 200 mg administered as an intravenous infusion over 30 minutes every 3 weeks until disease progression, unacceptable toxicity, or up to 24 months in patients without disease progression. For another example, PD-1 inhibitor nivolumab (OPTIVO), the recommended dosage for unresectable or metastatic melanoma as a single agent is either 240 mg every 2 weeks or 480 mg every 4 weeks administered as an intravenous infusion over 30 minutes until disease progression or unacceptable toxicity. For non-small cell lung cancer patients, the recommended dose of OPDIVO is either 240 mg every 2 weeks or 480 mg every 4 weeks administered as an intravenous infusion over 30 minutes until disease progression or unacceptable toxicity. For renal cell carcinoma patients, the recommended dose of OPDIVO as a single agent is either 240 mg every 2 weeks or 480 mg every 4 weeks administered as an intravenous infusion over 30 minutes until disease progression or unacceptable toxicity. For patients with classic Hodgkin's lymphoma, the recommended dose of OPDIVO is either 240 mg every 2 weeks or 480 mg every 4 weeks administered as an intravenous infusion over 30 minutes until disease progression or unacceptable toxicity. For patients with recurrent or metastatic squamous cell carcinoma of the head and neck, the recommended dose of OPDIVO is either 240 mg every 2 weeks or 480 mg every 4 weeks administered as an intravenous infusion over 30 minutes until disease progression or unacceptable toxicity. For patients with urothelial carcinoma, the recommended dose of OPDIVO is either 240 mg every 2 weeks or 480 mg every 4 weeks administered as an intravenous infusion over 30 minutes until disease progression or unacceptable toxicity. For patients with colorectal carcinoma, the recommended dose of OPDIVO is 240 mg every 2 weeks administered as an intravenous infusion over 30 minutes until disease progression or unacceptable toxicity. For patients with hepatocellular carcinoma, the recommended dose of OPDIVO is either 240 mg every 2 weeks or 480 mg every 4 weeks administered as an intravenous infusion over 30 minutes until disease progression or unacceptable toxicity.

For example, PD-1 inhibitor cemiplimab-rwlc (LIBTAYO), for patients with metastatic cutaneous squamous cell carcinoma (CSCC) or locally advanced CSCC who are not candidates for curative surgery or curative radiation the recommended dosage is 350 mg as an intravenous infusion over 30 minutes every 3 weeks.

For PD-L1 inhibitor avelumab (BAVENCIO), the recommended dosage for patients with metastatic Merkel cell carcinoma or locally advanced or metastatic urothelial carcinoma is 800 mg administered as an intravenous infusion over 60 minutes every 2 weeks until disease progression or unacceptable toxicity.

For PD-L1 inhibitor atezolizumab (TECENTRIQ), the recommended dosage for patients with advanced or metastatic urothelial carcinoma or metastatic non-small cell lung cancer is 1200 mg as an intravenous infusion over 60 minutes every 3 weeks. If the first infusion is tolerated, all subsequent infusions may be delivered over 30 minutes.

For PD-L1 inhibitor durvalumab (IMFINZI), the recommended dosage for patients with locally advanced or metastatic urothelial carcinoma is 10 mg/kg as an intravenous infusion over 60 minutes every 2 weeks.

In some embodiments of the methods with an anti-CTLA-4 conjugate and an immune modulatory agent, the immune modulatory agent is administered at the recommended dose and/or schedule of administration. In some embodiments, an immune modulatory agent can be administered in the methods herein at a dose lower than the recommended amount or on an alternate schedule, such as when anti-CTLA-4 conjugate sensitizes a tumor or lesion or the TME to the immune modulatory agent and/or when the combination of an anti-CTLA-4 conjugate and an immune modulatory agent results in a synergistic response.

### VI. DEVICES AND ILLUMINATION METHODS FOR USE WITH THE ANTI-CTLA-4 CONJUGATE METHODS AND COMPOSITIONS

In some aspects, devices that can be used with the methods and compositions herein include light diffusing devices that provide illumination at a wavelength (or wavelengths) of light wavelength suitable for use with the dye conjugate composition, such as a phthalocyanine dye conjugate (e.g., an anti-CTLA-4 conjugate such as those described herein). Illumination devices can include a light source (for example, a laser), and a means of conveying the light to the area or the site of interest (for example, one or more fibers to illuminate an isolated area of a subject or an isolated lesion or tumor).

In some embodiments, the cells, such as a tumor, are irradiated with a therapeutic dose of radiation at a wavelength within a range from or from about 400 nm to about 900 nm, such as from or from about 500 nm to about 900 nm, such as from or from about 600 nm to about 850 nm, such as from or from about 600 nm to about 740 nm, such as from about 660 nm to about 740 nm, from about 660 nm to about 710 nm, from about 660 nm to about 700 nm, from about 670 nm to about 690 nm, from about 680 nm to about 740 nm, or from about 690 nm to about 710 nm. In some embodiments, the cells, such as a tumor, are irradiated with a therapeutic dose of radiation at a wavelength of 600 nm to 850 nm, such as 660 nm to 740 nm. In some embodiments, the cells, such as a tumor, is irradiated at a wavelength of at least or about at least 600 nm, 620 nm, 640 nm, 660 nm, 680, nm, 700 nm, 720 nm or 740 nm, such as 690±50 nm, for example about 680 nm.

In some embodiments of the methods and uses provided herein, illumination is carried out using cylindrical diffusing fibers that includes a diffuser length of 0.5 cm to 10 cm and spaced 1.8±0.2 cm apart. In some embodiments, the light illumination dose is from or from about 20 J/cm fiber length to about 500 J/cm fiber length. In some embodiments, the tumor is greater than 10 mm deep or is a subcutaneous tumor.

In some embodiments, the provided methods include illuminating an interstitial tumor in a subject with cylindrical diffusing fibers that includes a diffuser length of 0.5 cm to 10 cm and spaced 1.8±0.2 cm apart with a light dose of or about 100 J/cm fiber length or with a fluence rate of or about 400 mW/cm. In some embodiments, the tumor is greater than 10 mm deep or is a subcutaneous tumor. In some embodiments, the cylindrical diffusing fibers are placed in a catheter positioned in the tumor 1.8±0.2 cm apart. In some embodiments, the catheter is optically transparent.

In some embodiments, the cells, such as a tumor, are illuminated at a dose of at least 1 J/cm², such as at least 10 J/cm², at least 30 J/cm², at least 50 J/cm², at least 100 J/cm², or at least 500 J/cm². In some embodiments, the dose of illumination is from or from about 1 to about J/cm², from about 1 to about 500 J/cm², from about 5 to about 200 J/cm², from about 10 to about 100 J/cm², or from about 10 to about 50 J/cm². In some embodiments, the cells, such as a tumor, are irradiated at a dose of at least or at least about 2 J/cm², 5 J/cm², 10 J/cm², 25 J/cm², 50 J/cm², 75 J/cm², 100 J/cm², 150 J/cm², 200 J/cm², 300 J/cm², 400 J/cm², or 500 J/cm².

In some embodiments, the lesion is a tumor that is a superficial tumor. In some embodiments, the tumor is less than 10 mm thick. In some embodiments, illumination is carried out using a microlens-tipped fiber for surface illumination. In some embodiments, the light illumination dose is from or from about 5 J/cm² to about 200 J/cm².

In some embodiments, the cells, such as a tumor, are illuminated at a dose of at least 1 J/cm fiber length, such as at least 10 J/cm fiber length, at least 50 J/cm fiber length, at least 100 J/cm fiber length, at least 250 J/cm fiber length, or at least 500 J/cm fiber length. In some embodiments, the dose of irradiation is from or from about 1 to about 1000 J/cm fiber length, from about 1 to about 500 J/cm fiber length, from about 2 to about 500 J/cm fiber length, from about 50 to about 300 J/cm fiber length, from about 10 to about 100 J/cm fiber length, or from about 10 to about 50 J/cm fiber length. In some embodiments, the cells, such as a tumor, are irradiated at a dose of at least or at least about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

In some embodiments, the provided methods include illuminating an superficial tumor in a subject with a microlens-tipped fiber for surface illumination with a light dose of from or from about 5 J/cm² to about 200 J/cm². In some embodiments, the light illumination dose is or is about 50 J/cm².

In some embodiments, the dose of irradiation or illumination in a human subject is from or from about 1 to about 400 J/cm², from about 2 to about 400 J/cm², from about 1 to about 300 J/cm², from about 10 to about 100 J/cm² or from about 10 to about 50 J/cm², from about such as is at least or at least about or is or within or within about or is or is about 10 J/cm², at least 30 J/cm², at least 50 J/cm², at least 100 J/cm². In some embodiments, the dose of illumination in a human subject is from or from about 1 to 300 J/cm fiber length, 10 to 100 J/cm fiber length or 10 to 50 J/cm fiber length, such as is at least or at least about or is or within or within about or is or is about 10 J/cm fiber length, at least 30 J/cm fiber length, at least 50 J/cm fiber length, at least 100 J/cm fiber length. In some cases, it is found that a dose of illumination in a human subject to achieve PIT can be less than is necessary for PIT in a mouse. For example, in some cases, 50 J/cm² (50 J/cm²) light dosimetry in an in vivo tumor mouse model is not effective for PIT, which is in contrast to what we can be observed in the clinic with human patients.

In some embodiments, the dose of illumination following administration of the composition comprising the phthalocyanine dye-targeting molecule conjugate is at least 1 J/cm² or 1 J/cm of fiber length at a wavelength of 660-740 nm, for example, at least 10 J/cm² or 10 J/cm of fiber length at a wavelength of 660-740 nm, at least 50 J/cm² or 50 J/cm of fiber length at a wavelength of 660-740 nm, or at least 100 J/cm² or 100 J/cm of fiber length at a wavelength of 660-740 nm, for example 1.0 to 500 J/cm² or 1.0 to 500 J/cm of fiber length at a wavelength of 660-740 nm. In some embodiments, the wavelength is 660-710 nm. In some embodiments, the dose of illumination following administration of the composition comprising the phthalocyanine dye-targeting molecule conjugate is at least 1.0 J/cm² or 1 J/cm of fiber length at a wavelength of 680 nm for example, at least 10 J/cm² or 10 J/cm of fiber length at a wavelength of 680 nm, at least 50 J/cm² or 50 J/cm of fiber length at a wavelength of 680 nm, or at least 100 J/cm² or 100 J/cm of fiber length at a wavelength of 680 nm, for example 1.0 to 500 J/cm² or 1.0 to 500 J/cm of fiber length at a wavelength of 680 nm. In some embodiments, multiple irradiations are performed, such as at least 2, at least 3, or at least 4 illuminations, such as 2, 3, 4, 5, 6, 7, 8, 9 or 10 separate administrations. Exemplary illumination after administration of the conjugates or compositions provided herein include illuminating the tumor at a wavelength of 660 nm to 740 nm at a dose of at least 1 J/cm² or 1 J/cm of fiber length.

In some embodiments, a light or laser may be applied to the dye molecules, such as cells containing the conjugate, for from about 5 seconds to about 5 minutes. For example, in some embodiments, the light or laser is applied for or for about 5, 10, 15, 20, 25, 30, 35, 40, 45 50 or 55 seconds, or for within a range between any of two such values, to activate the dye molecules. In some embodiments, the light or laser is applied for or for about 1, 1.5, 2, 2.5, 3, 3.5, 4, 4.5 or 5 minutes, or more, or within a range between any two of such values. In some embodiments, the length of time a light or laser is applied can vary depending, for example, on the energy, such as wattage, of the light or laser. For example, lights or lasers with a lower wattage may be applied for a longer period of time in order to activate the dye molecule.

In some embodiments, a light or laser may be applied about 30 minutes to about 48 hours after administering the conjugate. For example, in some embodiments, the light or laser is applied at or at about 30, 35, 40, 45, 50 or 55 minutes after administering the conjugate, or within a range between any two of such values. In some embodiments, the light or laser is applied at or at about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours after administering the conjugate, or is administered within a range between or between about any two of such values, such as, for example between about 20 hours to about 28 hours, or about 24 hours±4 hours. In some embodiments, the light or laser is applied between or between about 1 and 24 hours, such as between or between about 1 and 12 hours, 12 and 24 hours, 6 and 12 hours, or may be administered more than 24 hours following administration of the conjugate. In some embodiments, the light or laser is applied 36 or 48 hours after administering the conjugate. In some embodiments, the light or laser is applied at or at about 24 hours±4 hours after administering the conjugate.

In some embodiments, cells, or subjects, can be illuminated one or more times. Thus, illumination can be completed in a single day, or can be done repeatedly on multiple days with the same or a different dosage, such as illumination at least 2 different times, 3 different times, 4 different times 5 different times or 10 different times. In some embodiments, repeated illuminations may be done on the same day, on successive days, or every 1-3 days, every 3-7 days, every 1-2 weeks, every 2-4 weeks, every 1-2 months, or at even longer intervals.

In some embodiments, the dose or method of illumination differs depending on the type or morphology of the tumor.

In some embodiments, the lesion is a tumor that is a superficial tumor. In some embodiments, the tumor is less than 10 mm thick. In some embodiments, illumination is carried out using a microlens-tipped fiber for surface illumination. In some embodiments, the light illumination dose is from or from about 5 J/cm² to about 200 J/cm².

In some embodiments, the provided methods include illuminating an superficial tumor in a subject with a microlens-tipped fiber for surface illumination with a light dose of from or from about 5 J/cm² to about 200 J/cm², wherein the tumor is associated with a phototoxic agent that includes a targeting molecule bound to a cell surface molecule of the tumor. In some embodiments, the light irradiation dose is or is about 50 J/cm².

In some embodiments, the lesion is a tumor that is an interstitial tumor. In some embodiments, the tumor is greater than 10 mm deep or is a subcutaneous tumor. In some embodiments, illumination is carried out using cylindrical diffusing fibers that includes a diffuser length of 0.5 cm to 10 cm and spaced 1.8+-0.2 cm apart. In some embodiments, the light illumination dose is from or from about 20 J/cm fiber length to about 500 J/cm fiber length.

In some embodiments, the provided methods include illuminating an interstitial tumor in a subject with cylindrical diffusing fibers that includes a diffuser length of 0.5 cm to 10 cm and spaced 1.8±0.2 cm apart with a light dose of or about 100 J/cm fiber length or with a fluence rate of or about 400 mW/cm, wherein the tumor is associated with a phototoxic agent that includes a targeting molecule bound to a cell surface molecule of the tumor. In some embodiments, the tumor is greater than 10 mm deep or is a subcutaneous tumor. In some embodiments, the cylindrical diffusing fibers are placed in a catheter positioned in the tumor 1.8±0.2 cm apart. In some embodiments, the catheter is optically transparent.

In some embodiments, the illumination employs a device with “top hat” irradiance distribution profile, such as those described in WO2018/080952 and US20180239074.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “conjugate” refers to a targeting molecule linked directly or indirectly to a photoactivatable dye, such as those produced by chemical conjugates and those produced by any other methods. For example, a conjugate can refer to a phthalocyanine dye, such as a silicon-phthalocyanine dye (Si-phthalocyanine dye), such as an IR700 molecule, linked directly or indirectly to one or more targeting molecules, such as to a polypeptide binds to or targets to a cell surface protein. A targeting molecule can be a polypeptide, more than one polypeptide, an antibody or a chemical moiety.

As used herein an “anti-CTLA-4 conjugate” refers to a conjugate having a targeting molecule that binds to CTLA-4. An anti-CTLA-4 conjugate can have a targeting molecule that is an antibody, antigen-binding fragment, small molecule or other moiety that binds to CTLA-4.

As used herein, an “antibody” refers to a polypeptide comprising at least a light chain or heavy chain immunoglobulin variable region which specifically recognizes and binds an epitope of an antigen, such as a tumor-specific protein. Antibodies are composed of a heavy and a light chain, each of which has a variable region, termed the variable heavy (VH) region and the variable light (VL) region. Together, the VH region and the VL region are responsible for binding the antigen recognized by the antibody.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

“Specifically binds” refers to the ability of individual antibodies to specifically immunologically react with an antigen, such as a tumor-specific antigen, relative to binding to unrelated proteins, such as non-tumor proteins, for example β-actin. For example, a CTLA-4-specific binding agent binds substantially only the CTLA-4 protein in vitro or in vivo. As used herein, the term “tumor-specific binding agent” includes tumor-specific antibodies and other agents that bind substantially only to a tumor-specific protein in that preparation.

“Antibody-IR700 molecule” or “antibody-IR700 conjugate” refers to a molecule that includes both an antibody, such as a tumor-specific antibody, conjugated to IR700. In some examples the antibody is a humanized antibody (such as a humanized monoclonal antibody) that specifically binds to a surface protein on a cancer cell.

“Antigen” refers to a compound, composition, or substance that can stimulate the production of antibodies or a T cell response in an animal, including compositions (such as one that includes a tumor-specific protein) that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, an antigen includes a tumor-specific peptide (such as one found on the surface of a cancer cell) or immunogenic fragment thereof.

“Immune modulatory agent” and “immune modulatory therapy” refer to a therapeutic agent and treatment with such agent, respectively that modulates the immune system, such as a cytokine, an adjuvant and an immune checkpoint inhibitor.

“Immune checkpoint inhibitor” refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include PD-1/PD-L1 and CTLA-4/B7-1/B7-2. Some immune checkpoint inhibitors are used to treat cancer.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, “combination therapy” refers to a treatment in which a subject is given two or more therapeutic agents, such as at least two or at least three therapeutic agents, for treating a single disease. In some embodiments, each therapy can result in an independent pharmaceutical effect, and together can result in an additive or synergistic pharmaceutical effect.

As used herein, “treating” a subject with a disease or condition means that the subject's symptoms are partially or totally alleviated or remain static following treatment. Hence treating encompasses prophylaxis, therapy and/or cure. Prophylaxis refers to prevention of a potential disease and/or a prevention of worsening of symptoms or progression of a disease.

As used herein, “treatment” means any manner in which the symptoms of a condition, disorder or disease or other indication, are ameliorated or otherwise beneficially altered.

As used herein, “therapeutic effect” means an effect resulting from treatment of a subject that alters, typically improves or ameliorates the symptoms of a disease or condition or that cures a disease or condition.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, the term “subject” refers to an animal, including a mammal, such as a human being.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, a “tumor” refers to an abnormal mass of tissue that results when cells divide more than they should or do not die when they should. Tumors may be benign (not cancer), or malignant (cancer).

As used herein, a “lesion” refers to an area of abnormal tissue. A lesion may be benign (not cancer) or malignant (cancer).

As used herein, an “anti-cancer agent” refers to any molecules that are used for treatment to stop or prevent cancer. Examples may include, but are not limited to, small chemical molecules, antibodies, antibody conjugates, immunomodulators, or any combination thereof.

As used herein, a “suppressor cell” or an “immunosuppressor cell” refers to cells that are able to decrease or inhibit the function of immune effector cells such as CD8⁺ T effector cells. Example for suppressor cells may include, but are not limited to, regulatory T cells, M2 macrophages, myeloid derived suppressor cells, tumor associated fibroblasts, or cancer associated fibroblasts.

As used herein, an “immunosuppressive agent” refers to an agent that decreases the body's immune responses. It reduces the body's ability to fight infections and other diseases, such as cancer.

As used herein, “resistant to treatment” refers to that a disease or a pathological condition that is not responsive to a treatment, so that this treatment is not effective or does not show efficacy in treating this disease or pathological condition.

As used herein, “systemic immune response” refers to the ability of a subject's immune system to respond to an immunologic challenge or immunologic challenges, including those associated with a cancer, a tumor, or a cancerous lesion, in a systemic manner. Systemic immune response can include systemic response of the subject's adaptive immune system and/or innate immune system. Systemic immune response includes an immune response across different tissues, including the blood stream, lymph node, bone marrow, spleen and/or the tumor microenvironment, and in some cases, includes a coordinated response among the tissues and organs and various cells and factors of the tissues and organs.

As used herein, “local immune response” refers to the immune response in a tissue or an organ to an immunologic challenge or immunologic challenges including those associated with a cancer, a tumor, or a cancerous lesion. Local immune response can include the adaptive immune system and/or innate immune system. Local immunity includes immune response concurrently occurring at different tissues, including the blood stream, lymph node, bone marrow, spleen and/or the tumor microenvironment.

### VIII. EXEMPLARY EMBODIMENTS

Among the provided embodiments are:

1. A method of treating a tumor or lesion, comprising:

(a) identifying a subject having a tumor or lesion that is non-responsive to a prior therapeutic treatment;

(b) administering to the subject a conjugate comprising a phthalocyanine dye linked to a targeting molecule, wherein the targeting molecule binds to CTLA-4;

(c) after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and

(d) administering a first immune modulatory therapy to the subject;

wherein the growth and/or increase in volume of the tumor or lesion in the subject is inhibited or reduced.

2. The method of embodiment 1, wherein the prior therapeutic treatment comprises treatment with an immune modulatory agent, an immune checkpoint inhibitor, an anti-cancer agent, a therapeutic agent that acts against suppressor cells, and any combination thereof.

3. The method of embodiment 1 or embodiment 2, wherein the prior therapeutic treatment comprises treatment with a PD-1 inhibitor, a PD-L1 inhibitor, a CTLA-4 inhibitor, or any combination thereof.

4. The method of any of embodiments 1-3, wherein the prior therapeutic treatment comprises treatment with an antibody or antigen-binding fragment of the antibody.

5. The method of embodiment 4, wherein the antibody or antigen-binding fragment binds to PD-1, CTLA-4 or PD-L1.

6. The method of any of embodiments 1-5, wherein the first immune modulatory therapy is administered prior to administering the conjugate.

7. The method of embodiment 6, wherein the first immune modulatory therapy is administered between about 1-3 weeks prior to administering the conjugate.

8. The method of embodiment 6 or embodiment 7, wherein the first immune modulatory therapy is administered 1, 2, 3, 4, 5, or more than 5 times prior to administering the conjugate.

9. The method of any of embodiments 1-5, wherein the first immune modulatory therapy is administered concurrently with administering the conjugate.

10. The method of any of embodiments 1-5, wherein the first immune modulatory therapy is administered subsequent to administering the conjugate.

11. The method of embodiment 10, wherein the first immune modulatory therapy is administered 1, 2, 3, 4, 5, or more than 5 times subsequent to administering the conjugate.

12. The method of embodiment 10 or embodiment 11, wherein the first immune modulatory therapy is administered between about 1 day and 4 weeks after administering the conjugate.

13. The method of any of embodiments 1-5, wherein the first immune modulatory therapy is administered prior to administering the conjugate and administered at least one additional time subsequent to administering the conjugate.

14. The method of embodiment 13, wherein the first immune modulatory therapy is administered 1, 2 or 3 times prior to administering the conjugate.

15. The method of embodiment 13 or embodiment 14, wherein the first immune modulatory therapy is administered between about 1-3 weeks prior to administering the conjugate.

16. The method of any of embodiments 1-15, wherein the first immune modulatory therapy is an adjuvant for enhancing innate activation or an adjuvant for enhancing adaptive activation.

17. The method of any of embodiments 1-15, wherein the first immune modulatory therapy is a T cell agonist.

18. A method of treating tumor or lesion resistant to treatment with a prior immune checkpoint inhibitor, comprising:

(e) identifying a tumor or lesion in a subject that is non-responsive to or resistant to treatment with a prior immune checkpoint inhibitor;

(f) administering to the subject a conjugate comprising a phthalocyanine dye linked to a targeting molecule, wherein the targeting molecule binds to CTLA-4;

(g) after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and

(h) administering a first immune checkpoint inhibitor,

wherein the tumor or lesion exhibits sensitivity to the first immune checkpoint inhibitor.

19. The method of embodiment 18, wherein the prior immune checkpoint inhibitor is selected from the group consisting of a PD-1 inhibitor, a PD-L1 inhibitor or a CTLA-4 inhibitor.

20. The method of embodiment 18 or embodiment 19, wherein the subject comprises a second tumor or lesion that is not illuminated, and wherein the second tumor or lesion exhibits sensitivity to administering the first immune checkpoint inhibitor.

21. The method of embodiment 18 or embodiment 19, wherein the subject comprises metastatic tumor cells and wherein the metastatic tumor cells exhibit sensitivity to administering the first immune checkpoint inhibitor.

22. The method of any of embodiments 18-21, wherein sensitivity comprises a reduction or inhibition of tumor growth, a reduction in tumor cell metastasis, an increase in tumor cell killing, an increase in systemic immune response, an increase in new T cell priming, an increase in diversity of CD8 T cells or any combinations thereof.

23. The method of any of embodiments 18-22, wherein the first immune checkpoint inhibitor is a PD-1 inhibitor, a PD-L1 inhibitor or a CTLA-4 inhibitor.

24. The method of any of embodiments 18-23, wherein the first immune checkpoint inhibitor comprises an antibody or antigen-binding fragment of an antibody.

25. A method of provoking a systemic immune response comprising:

(i) administering to a subject a conjugate comprising a phthalocyanine dye linked to a targeting molecule, wherein the targeting molecule binds to CTLA-4;

(j) after administering the conjugate, illuminating at the site of a first tumor or first lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and

(k) administering a first immune modulatory therapy,

wherein following steps (i), (j), and (k), the subject exhibits at least one systemic response in a second tumor or second lesion distal to the illuminated site.

26. The method of embodiment 25, wherein the systemic response comprises a systemic immune responsive feature.

27. The method of embodiment 26, wherein the systemic immune responsive feature is selected from the group consisting of an increase in CD8 T cell infiltration, an increase in CD8 T cell activation, an increase in dendritic cell infiltration, an increase in dendritic cell activation, an increase in new T cell priming, an increase in T cell diversity or any combination thereof.

28. The method of embodiment 26, wherein the systemic immune responsive feature comprises an increase in one or more of a proinflammatory molecule, a proinflammatory cytokine, an immune cell activation marker, or T cell diversity.

29. The method of any of embodiments 26-28, wherein the systemic immune responsive feature is assessed from a blood sample obtained from the subject.

30. A method of provoking a local immune response comprising:

(l) administering to a subject a conjugate comprising a phthalocyanine dye linked to a targeting molecule, wherein the targeting molecule binds to CTLA-4;

(m) after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and

(n) administering a first immune modulatory therapy,

wherein following steps (l), (m), and (n), the subject exhibits at least one local response, and wherein the response is synergistic as compared to treatment with only the first immune modulatory therapy or as compared to treatment with the conjugate administration and illuminating alone.

31. The method of embodiment 30, wherein the local response comprises a local immune response.

32. The method of embodiment 31, wherein the local immune response is selected from the group consisting of intratumoral Treg depletion, an increase in intratumoral CD8 T cell infiltration, an increase in intratumoral CD8 T cell activation, a decrease in myeloid suppressive cells, a Type I interferon response and any combination thereof.

33. The method of embodiment 31, wherein the local immune response comprises an increase in the tumor or tumor microenvironment of an anti-immune cell type or an immune activation marker.

34. The method of any of embodiments 25-33, wherein the first immune modulatory therapy comprises treatment with a PD-1 inhibitor or a PD-L1 inhibitor.

35. The method of any of embodiments 25-34, wherein the first immune modulatory therapy comprises treatment with an antibody or antigen-binding fragment of an antibody.

36. The method of any of embodiments 25-33, wherein the first immune modulatory therapy is selected from the group consisting of an adjuvant for enhanced innate activation, an adjuvant for enhanced adaptive activation and a T cell agonist.

37. The method of any of embodiments 1-35, further comprising treatment with a second conjugate comprising a cancer targeting molecule conjugated to a phthalocyanine dye, and wherein at least one illuminating step is performed subsequent to administering the second conjugate.

38. A method of treating a tumor or lesion, comprising:

(o) identifying a cold tumor or lesion in a subject;

(p) administering to the subject a conjugate comprising a phthalocyanine dye linked to a targeting molecule, wherein the targeting molecule binds to CTLA-4; and

(q) after administering the conjugate, illuminating the tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length,

wherein the growth and/or increase in volume of the cold tumor or lesion in the subject is inhibited or reduced.

39. The method of embodiment 38, wherein the inhibition of tumor growth is enhanced as compared to treatment with a naked or unconjugated CTLA-4 antibody.

40. The method of embodiment 38 or embodiment 39, wherein the cold tumor or lesion is identified by a high mutational burden or a tumor immune score.

41. The method of embodiment 38 or embodiment 39, wherein the cold tumor or lesion is identified by status of expression of a PD-1 or a PD-L1 marker.

42. The method of embodiment 38 or embodiment 39, wherein the cold tumor or lesion is identified based on failure of the tumor or lesion to respond to a PD-1 inhibitor or an PD-L1 inhibitor.

43. The method of any of embodiments 38-42, wherein the cold tumor or lesion is identified by a liquid biopsy or a tissue biopsy.

44. The method of any of embodiments 38-43, wherein Treg cells are rapidly depleted in the tumor or tumor microenvironment following the illuminating step.

45. The method of any of embodiments 38-44, wherein necrosis of the tumor cells occurs following the illuminating step.

46. The method of any of embodiments 1-45, wherein the targeting molecule comprises an anti-CTLA-4 antibody or antigen-binding fragment thereof.

47. The method of embodiment 46, wherein the anti-CTLA-4 antibody is selected from the group consisting of ipilimumab (YERVOY), tremelimumab, AGEN1181, AGEN1884, ADU-1064, BCD-145, and BCD-217.

48. The method of any of embodiments 1-47, wherein the phthalocyanine dye is a Si-phthalocyanine dye.

49. The method of embodiment 48, wherein the Si-phthalocyanine dye is IR700.

50. The method of any of embodiments 1-49, wherein the first immune modulatory therapy or the first immune checkpoint inhibitor comprises treatment with an anti-PD-1 antibody selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

51. The method of any of embodiments 1-49, wherein the first immune modulatory therapy or the first immune checkpoint inhibitor comprises treatment with an anti-PD-L1 antibody selected from the group consisting of atezolizumab (MPDL3280A, TECENTRIQ, RG7446), avelumab (BAVENCIO, MSB0010718C; M7824), durvalumab (MEDI4736, IMFINZI), LDP, NM-01, STI-3031 (IMC-001; STI-A1015), KN035, LY3300054, M7824 (MSB0011359C), BMS-936559, MSB2311, BCD-135, BGB-A333, CBT-502 (TQB-2450), cosibelimab (CK-301), CS1001 (WPB3155), FAZ053, MDX-1105, SHR-1316 (HTI-1088), TG-1501, ZKAB001 (STI-A1014), INBRX-105, MCLA-145, KN046, LY3415244, REGN3504, and HLX20.

52. The method of any of embodiments 1-51, wherein the illuminating step is carried out between 30 minutes and 96 hours after administering the conjugate.

53. The method of any of embodiments 1-52, wherein the illuminating step is carried out 24 hours±4 hours after administering the conjugate.

54. The method of any of embodiments 1-53, wherein the illuminating step is carried out at a wavelength of 690±40 nm.

55. The method of any of embodiments 1-54, wherein the illuminating step is carried out at a dose of or about of 50 J/cm² or 100 J/cm of fiber length.

56. The method of any of embodiments 1-55, wherein the administration of the conjugate is repeated one or more times, optionally wherein after each repeated administration of the conjugate, the illuminating step is repeated.

57. The method of any of embodiments 1-56, further comprising administering an additional therapeutic agent or anti-cancer treatment.

58. The method of any of embodiments 1-57, wherein the tumor or lesion is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

59. The method of any of embodiments 1-58, wherein the conjugate provides an effect independent of the number or activity of systemic regulatory T cells.

60. The method of any of embodiments 1-58, wherein the method results in a substantial increase in the number or frequency of intratumoral cytotoxic T effector cells, natural killer (NK) cells, other immune effector cells, or any combination thereof.

61. The method of any of embodiments 1-58, wherein the method results in in a substantial increase in the activity or function of intratumoral cytotoxic T effector cells, natural killer (NK) cells, other immune effector cells, or any combination thereof.

62. The method of any of embodiments 1-58, wherein the method results in a substantial decrease in the number or frequency and/or activity or function of an intratumoral suppressor cell.

63. The method of embodiment 62, wherein the intratumoral suppressor cell is selected from the group consisting of regulatory T cells, type II natural killer T cells, M2 macrophages, tumor associated fibroblast, myeloid-derived suppressor cell, or any combination thereof.

64. A method of treating a tumor or a lesion that is non-responsive to or resistant to a prior immune checkpoint inhibitor therapy, the method comprising:

(a) identifying a tumor or a lesion in a subject that is non-responsive to or resistant to treatment with a prior immune checkpoint inhibitor;

(b) administering to the subject a conjugate comprising a phthalocyanine dye linked to a targeting molecule that binds to CTLA-4;

(c) after administering the conjugate, illuminating the tumor or the lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and

(d) administering a first immune checkpoint inhibitor, wherein the tumor or the lesion exhibits sensitivity to the first immune checkpoint inhibitor.

65. The method of embodiment 64, wherein sensitivity to the first immune checkpoint inhibitor comprises a reduction in volume, dimensions or mass of the tumor or the lesion, a less than 20% increase in volume or dimensions of the tumor or the lesion, or a reduction in the number of tumor cells.

66. The method of embodiment 64, wherein sensitivity to the first immune checkpoint inhibitor comprises a reduction in tumor cell metastasis, an increase in tumor cell killing, an increase in systemic immune response, an increase in new T cell priming, an increase in diversity of CD8⁺ T cells or any combinations thereof.

67. The method of embodiment 66, wherein sensitivity to the first immune checkpoint inhibitor comprises an increase in systemic immune response, and the systemic immune response is measured by one or more of a cytotoxic T lymphocyte (CTL) activity assay, an intratumoral T cell exhaustion assay, an intratumoral effector T cell expansion assay, a T cell receptor diversity assay, an activated CD8⁺ T cell assay, a circulating regulatory T cell (Treg) assay, an intratumoral Treg assay, or a CD8⁺ Tcell:Treg assay.

68. The method of any of embodiments 64-67, wherein the tumor or the lesion that is non-responsive or resistant is identified by a high mutational burden or a tumor immune score.

69. The method of any of embodiments 64-67, wherein the tumor or the lesion that is non-responsive or resistant is identified by status of expression of a PD-1 or a PD-L1 biomarker.

70. The method of any of embodiments 64-69, wherein the tumor or the lesion that is non-responsive or resistant is identified by a liquid biopsy or a tissue biopsy.

71. The method of any of embodiments 64-70, wherein the treatment with the prior immune checkpoint inhibitor comprises treatment with a PD-1 inhibitor, a PD-L1 inhibitor or a CTLA-4 inhibitor.

72. The method of any of embodiments 64-71, wherein the treatment with the prior immune checkpoint inhibitor comprises treatment with an anti-PD-1 antibody or antigen-binding fragment thereof.

73. The method of embodiment 72, wherein the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

74. A method of provoking a systemic immune response, the method comprising:

(a) administering to a subject a conjugate comprising a phthalocyanine dye linked to a targeting molecule that binds to CTLA-4;

(b) after administering the conjugate, illuminating at the site of a first tumor or a first lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and

(c) administering a first immune checkpoint inhibitor, wherein following steps (a), (b), and (c), the subject exhibits at least one systemic immune responsive feature in a location distal to the illuminated site.

75. The method of embodiment 74, wherein the at least one systemic immune responsive feature is selected from the group consisting of an increase in CD8⁺ T cell infiltration, an increase in CD8⁺ T cell activation, an increase in the CD8⁺:Treg ratio, an increase in natural killer cell infiltration, an increase in natural killer cell activation, an increase in dendritic cell infiltration, an increase in dendritic cell activation, an increase in new T cell priming, an increase in T cell diversity, and any combination thereof. 76. The method of embodiment 74, wherein the at least one systemic immune responsive feature comprises an increase in one or more of a proinflammatory molecule, a proinflammatory cytokine, or an immune cell activation marker.

77. The method of any of embodiments 74-76, wherein the at least one systemic immune responsive feature is assessed from a blood sample obtained from the subject.

78. The method of any of embodiments 74-77, wherein the location distal to the illuminated site is a second tumor or a second lesion that is not illuminated.

79. A method of provoking a local immune response comprising:

(a) administering to a subject a conjugate comprising a phthalocyanine dye linked to a targeting molecule that binds to CTLA-4;

(b) after administering the conjugate, illuminating the tumor or the lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and

(c) administering a first immune checkpoint inhibitor, wherein following steps (a), (b), and (c), the subject exhibits at least one local immune responsive feature, and wherein the at least one local immune responsive feature is synergistic as compared to administering only the first immune checkpoint inhibitor or as compared to treatment only with the conjugate and the illuminating step.

80. The method of embodiment 79, wherein the at least one local immune responsive feature is selected from the group consisting of intratumoral Treg depletion, an increase in intratumoral CD8 T cell infiltration, an increase in intratumoral CD8 T cell activation, an increase in the intratumoral CD8⁺:Treg ratio, an increase in intratumoral natural killer cell infiltration, an increase in intratumoral natural killer cell activation, a decrease in myeloid suppressive cells, a Type I interferon response, and any combination thereof.

81. The method of embodiment 79, wherein the at least one local immune responsive feature comprises an increase in an anti-immune cell type or an immune activation marker in the tumor or tumor microenvironment.

82. The method of any of embodiments 64-81, wherein the targeting molecule comprises an anti-CTLA-4 antibody or an antigen binding fragment thereof.

83. The method of embodiment 82, wherein the anti-CTLA-4 antibody is selected from the group consisting of ipilimumab (YERVOY), tremelimumab, AGEN1181, AGEN1884, ADU-1064, BCD-145, CBT-509, and BCD-217.

84. The method of any of embodiments 64-83, wherein the first immune checkpoint inhibitor comprises an anti-PD-1 antibody or antigen-binding fragment thereof.

85. The method of embodiment 84, wherein the first immune checkpoint inhibitor is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011), and antigen-binding fragments thereof.

86. The method of any of embodiments 64-85, wherein the first immune checkpoint inhibitor is administered concurrently with the administering the conjugate.

87. The method of any of embodiments 64-85, wherein the first immune checkpoint inhibitor is administered within 24 hours of administering the conjugate.

88. The method of any of embodiments 64-85, wherein the first immune checkpoint inhibitor is administered prior to administering the conjugate.

89. The method of embodiment 88, wherein the first immune checkpoint inhibitor is administered between about 1-3 weeks prior to administering the conjugate.

90. The method of embodiment 88 or embodiment 89, wherein the first immune checkpoint inhibitor is administered 1, 2, 3, 4, 5 times, or more than 5 times prior to administering the conjugate.

91. The method of any of embodiments 64-90, further comprising administering the first immune checkpoint inhibitor subsequent to administering the conjugate.

92. The method of embodiment 91, wherein the first immune checkpoint inhibitor is administered 1, 2, 3, 4, 5 times, or more than 5 times subsequent to administering the conjugate.

93. The method of embodiment 91 or embodiment 92, wherein the first immune checkpoint inhibitor is administered between about 1 day and about 4 weeks after administering the conjugate.

94. The method of any of embodiments 64-73 and 82-93, wherein the subject exhibits progressive disease or a stable disease following treatment with a prior immune checkpoint inhibitor.

95. The method of any of embodiments 64-73 and 82-93, wherein the tumor or the lesion that is non-responsive to or resistant to a prior immune checkpoint inhibitor therapy comprises a tumor or a lesion that exhibits a lack of reduction in volume, dimensions or mass of the tumor or the lesion, more than 20% increase in volume or dimensions of the tumor or the lesion, or an increase in the number of tumor cells, or a metastases.

96. The method of any of embodiments 64-95, wherein the subject comprises a second tumor or lesion that is not illuminated, and wherein the second tumor or lesion exhibits sensitivity to administering the first immune checkpoint inhibitor.

97. The method of any of embodiments 64-95, wherein the subject comprises metastatic tumor cells and wherein the metastatic tumor cells exhibit sensitivity to administering the first immune checkpoint inhibitor.

98. The method of any of embodiments 64-97, wherein the subject does not experience a substantial reduction in systemic Treg cells.

99. The method of any of embodiments 64-98, wherein the subject exhibits a response at a site distal to the illuminated tumor or lesion, wherein the response is selected from the group consisting of an increase in CD8⁺ T cell infiltration, an increase in CD8⁺ T cell activation, an increase in the intratumoral CD8⁺:Treg ratio, an increase in intratumoral natural killer cell infiltration, an increase in intratumoral natural killer cell activation, an increase in dendritic cell infiltration, an increase in dendritic cell activation, an increase in new T cell priming, an increase in T cell diversity, increase in one or more of a proinflammatory molecule, a proinflammatory cytokine, an immune cell activation marker, and any combination thereof.

100. The method of any of embodiments 64-99, wherein the method results in a substantial decrease in the number, the frequency, the activity and/or the function of an intratumoral suppressor cell.

101. The method of embodiment 100, wherein the intratumoral suppressor cell is selected from the group consisting of regulatory T cells, type II natural killer T cells, M2 macrophages, tumor associated fibroblast, myeloid-derived suppressor cell, and any combination thereof.

102. The method of any of embodiments 64-101, wherein the method results in a substantial increase in the number or the frequency of intratumoral cytotoxic T effector cells, natural killer (NK) cells, other immune effector cells, or any combination thereof.

103. The method of any of embodiments 64-102, wherein the method results in in a substantial increase in the activity or the function of intratumoral cytotoxic T effector cells, natural killer (NK) cells, other immune effector cells, or any combination thereof.

104. The method of any of embodiments 64-103, wherein necrosis of the tumor or the lesion occurs following the illuminating step.

105. The method of any of embodiments 64-104, wherein the phthalocyanine dye is a Si-phthalocyanine dye.

106. The method of embodiment 105, wherein the Si-phthalocyanine dye is IR700.

107. The method of any of embodiments 64-106, wherein the illuminating step is carried out between 30 minutes and 96 hours after administering the conjugate.

108. The method of any of embodiments 64-107, wherein the illuminating step is carried out 24 hours±4 hours after administering the conjugate.

109. The method of any of embodiments 64-108, wherein the illuminating step is carried out at a wavelength of 690±40 nm.

110. The method of any of embodiments 64-109, wherein the illuminating step is carried out at a dose of or about of 50 J/cm² or 100 J/cm of fiber length.

111. The method of any of embodiments 64-110, wherein the administration of the conjugate is repeated one or more times, optionally wherein after each repeated administration of the conjugate, the illuminating step is repeated.

112. The method of any of embodiments 64-111, further comprising administering an additional therapeutic agent or anti-cancer treatment.

113. The method of any of embodiments 64-112, wherein the tumor or the lesion is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

### IX. EXAMPLES

The following examples are included for illustrative purposes only and are not intended to limit the scope of the invention.

### Example 1: Generation of IRDye 700-Conjugated Anti-CTLA-4 Antibody

This example describes a method for preparing a conjugate containing IRDye 700DX (IR700) linked to anti-CTLA-4 antibody 9H10, thus producing 9H10-IRDye 700DX (9H10-IR700).

Antibody 9H10, a Syrian Hamster IgG monoclonal antibody (mAb) directed against mouse CTLA-4, was incubated (1 mg, 6.8 nmol) with IRDye 700DX NHS Ester (IR700; LI-COR Bioscience, Lincoln, Nebr.) (66.8 μg, 34.2 nmol, 5 mmol/L in DMSO) in 0.1 mol/L Na₂HPO₄ (pH 8.5) at room temperature for 30 to 120 min. The mixture was purified using a Sephadex G50 column (PD-10; GE Healthcare, Piscataway, N.J.). The protein concentration was determined with Coomassie Plus protein assay kit (Pierce Biotechnology, Rockford, Ill.) by measuring the absorption at 595 nm with a UV-Vis system (8453 Value System; Agilent Technologies, Palo Alto, Calif.). The concentration of IR700 was measured by absorption with the UV-Vis system to confirm the number of fluorophore molecules conjugated to each 9H10 molecule. The number of IR700 per 9H10 was about 3.

The purity of the 9H10-IR700 conjugate was confirmed by analytical size-exclusion HPLC (SE-HPLC) and sodium dodecyl sulfate polyacrylamide gel electrophoresis (SDS-PAGE). SE-HPLC was performed using a Beckman System Gold (Fullerton, Calif.) equipped with model 126 solvent delivery module, a model 168 UV detector, and a JASCO fluorescence detector (excitation 689 nm and emission at 700 nm) controlled by 32 Karat software. SE chromatography was performed on a TSK gel G2000SW×1 (Tosoh Bioscience LLC, Montgomeryville, Pa.) eluted for 45 minutes using phosphate buffered saline (PBS) at 0.5 mL/min. SDS-PAGE was performed with a 4% to 20% gradient polyacrylamide gel (Invitrogen, Carlsbad, Calif.). After separating the proteins, fluorescence intensity was analyzed with a Fujifilm FLA-5100 fluorescence scanner (Valhalla, N.Y.) with an internal laser of 670 nm for excitation and 705 nm long pass filter for emission. The fluorescence intensity of each band was analyzed with Multigage software (Fujifilm). The gels were then stained with Colloidal Blue Staining Kit (Invitrogen), and digitally scanned. The protein concentration in each band was analyzed with ImageJ software. The 9H10-IR700 preparation demonstrated strong association and contained no detectable mAb aggregates as determined by HPLC and SDS-PAGE.

To determine the in vitro binding characteristics of IR700 conjugates, ¹²⁵I labeling of the conjugates using the Indo-Gen procedure was performed. Minimal loss of mAb with IR700 conjugation was confirmed. Immunoreactivity assay was performed as described previously. Briefly, after trypsinization, 2×10⁶ of tumor cells were resuspended in PBS containing 1% bovine serum albumin (BSA). ¹²⁵I-9H10-IR700 (1 mCi, 0.2 μg) was added and incubated for 1 h on ice. Cells were washed, pelleted, the supernatant decanted, and counted in a 2470 Wizard gamma-counter (Perkin Elmer, Shelton, Conn.). Nonspecific binding to the cells was examined under conditions of antibody excess (200 μg of non-labeled 9H10).

### Example 2: Anti-CTLA-4-IR700 Photoimmunotherapy (PIT) Inhibits the Growth of Tumors with Reduced Immunoresponsiveness

This example describes the inhibitory effect of anti-CTLA-4-IR700 photoimmunotherapy (PIT) on the growth of tumors with reduced immunoresponsiveness.

BALB/c mice at age of 6-8 weeks were inoculated with 1×10⁶ CT26-EphA2 clone c4D10 murine colon carcinoma cells per mouse subcutaneously on the right hind flank. When allograft tumors grew to a size about 250-350 mm³ (11 days after tumor cell inoculation), mice were administered with saline (100 μL), “naked” unconjugated anti-CTLA-4 antibody 9H10 (100 μg), or 9H10-IR700 (anti-CTLA-4-IR700) conjugate that was generated substantially as described in Example 1 above (100 μg). Animals receiving “naked” unconjugated anti-CTLA-4 were administered additional doses (100 μg) of the antibody 15 and 19 days after tumor cell inoculation. Twenty-four hours after administration of anti-CTLA-4-IR700, tumors in the PIT group were illuminated at 690 nm and a dosage of 150 J/cm². The tumor growth was observed over 21 days, and tumor volume was calculated using a formula: tumor volume=(width×length)×height/2.

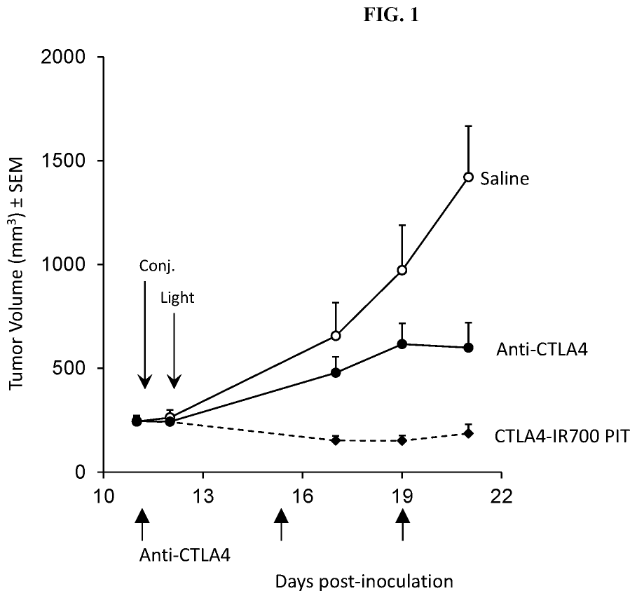

When the tumors grew to a size about 250-350 mm³ they developed an immunosuppression phenotype: e.g., the number of intratumoral cytotoxic CD8⁺ T cells decreased and the number of intratumoral regulatory T cells (immune suppressor cells) increased (data not shown). As shown in FIG. 1, when mice were treated with multiple administrations of the anti-CTLA-4 antibody (anti-CTLA4), the growth of tumors was substantially inhibited in comparison to the saline control group (closed circles vs. open circles). In mice treated with a single cycle of anti-CTLA-4-IR700 PIT (CTLA4-IR700 PIT), the growth of tumors was further inhibited (FIG. 1; closed diamonds).

### Example 3: Anti-CTLA-4 PIT Induces Anti-Cancer Response in Distal Tumors

This example describes the inhibitory effect of anti-CTLA-4 PIT on the growth of distal tumors that are not directly illuminated.

BALB/c mice were inoculated with 1×10⁶ CT26-EphA2 clone c4D10 murine colon carcinoma cells, or 1×10⁶ MCA205 murine fibrosarcoma cells, per mouse subcutaneously on both the right and left hind flanks. When allograft tumors on both sides grew to volumes of about 250-350 mm³ for CT26-EphA2 cells, or about 150 mm³ for MCA205 cells, mice were administered saline (100 μL) or anti-CTLA-4 (9H10)-IR700 conjugate (100 μg). Twenty-four hours after administration of the conjugate, tumors in the right flank in the anti-CTLA-4 PIT group were illuminated at 690 nm at a dosage of 150 J/cm² for CT26-EphA2 tumors or 150 J/cm² for MCA205 tumors, while tumors in the left flank were shielded from illumination. The growth of the illuminated tumor (target tumor) and the non-illuminated tumor (distal tumor) was observed time (19-20 days), and tumor volume was calculated using the formula: tumor volume=(width×length)×height/2.

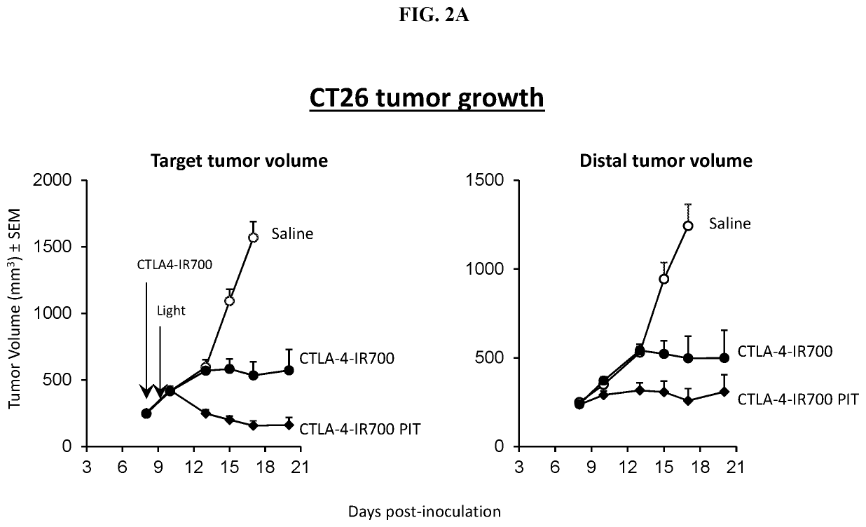

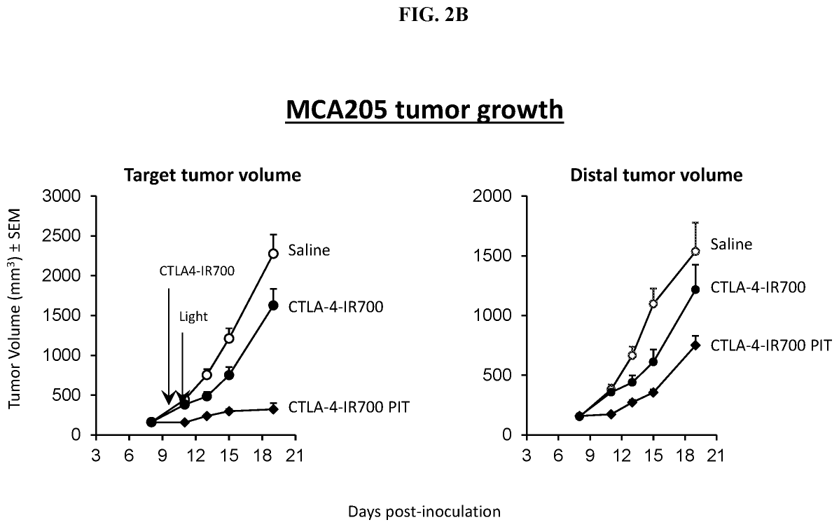

As shown in FIGS. 2A and 2B, target tumors (left panels) and distal tumors (right panels) treated with anti-CTLA-4 PIT exhibited tumor growth inhibition compared to saline-treated or anti-CTLA-4-IR700 conjugate-treated (no PIT) tumors for both tumor cell types. Mice administered the anti-CTLA-4-IR700 conjugate alone (without illumination) also exhibited reduced target tumor (FIGS. 2A and 2B; left panels) and distal tumor (FIGS. 2A and 2B; right panels) growth compared to saline controls, but the conjugate alone was less effective in inhibiting target (FIGS. 2A and 2B; left panels) and distal tumor (FIGS. 2A and 2B; right panels) growth in comparison to anti-CTLA-4-PIT in both tumor cell types. These data support the finding that anti-CTLA-4 PIT is able to induce a local and systemic immune response and exhibit an abscopal effect, e.g., inhibition of distal (non-illuminated) tumor growth, in comparison to treatment with the anti-CTLA-4-IR700 conjugate alone.

### Example 4: Resistance of “Cold” Tumors to Anti-CTLA-4 and Anti-PD-1 Therapies

This example describes the resistance of “cold” tumors, i.e. tumors with diminished immunoresponsiveness, to anti-CTLA-4 antibody 9H10 monotherapy or the combination of 9H10 and anti-PD-1 antibody RMP1-14 therapies.

To produce a murine tumor model with diminished immunoresponsiveness, we used 4T1 murine mammary carcinoma cell allograft tumors. It has been shown that in 4T1 tumors, the numbers and/or activities of intratumoral cytotoxic cells (e.g., CD8+T effector cells) are substantially reduced or absent, thus making this type of tumor “cold” (Mosely et al., (2017) Cancer Immunol Res. 5(1):29-41).

BALB/c mice at age of 6-8 weeks were inoculated with 1×10⁶ 4 T1 cells per mouse subcutaneously on the right hind flank. When the allograft tumors reached an approximate average volume of 150 mm³ (7 days after tumor cell inoculation), mice were administered saline (100 μL), saline plus anti-PD-1 antibody clone RMP1-14 (100 μg), 9H10-IR700 (anti-CTLA-4-IR700) conjugate that was generated substantially as described in Example 1 above (100 μg), or the combination of 9H10-IR700 (anti-CTLA-4-IR700) conjugate and RMP1-14 (100 μg each). The anti-CTLA-4-IR700 conjugate was administered at day 7 and RMP1-14 at days 7, 9, 11, and 14. The tumor growth was observed over 26 days, and tumor volume was calculated using a formula: tumor volume=(width×length)×height/2.

The results showed that the “cold” 4T1 tumors were resistant to any of the treatments administered to the mice. In comparison to the control group (saline), administration of the anti-CTLA-4-IR700 conjugate alone, or in combination with anti-PD-1 antibody, only partially reduced the growth of tumors (FIG. 3).

### Example 5: Resistance of Cold Tumors to Anti-CTLA-4 PIT

This example demonstrates the resistance of “cold” tumors, i.e. tumors with diminished immunoresponsiveness, to anti-CTLA-4 PIT.

BALB/c mice at age of 6-8 weeks were inoculated with 1×10⁶ 4 T1 cells per mouse subcutaneously on the right hind flank. When allograft tumors grew to a volume of about 150 mm³ (6 days after tumor cell inoculation), mice were administered saline (100 μL) or anti-CTLA-4 antibody 9H10-IR700 conjugate (100 μg). Twenty-four hours after administration of the conjugate, tumors in the anti-CTLA-4 PIT group were illuminated at 690 nm at a dosage of 150 J/cm². Survival and tumor growth were observed over 26 days, and the tumor volume was calculated using a formula: tumor volume=(width×length)×height/2.

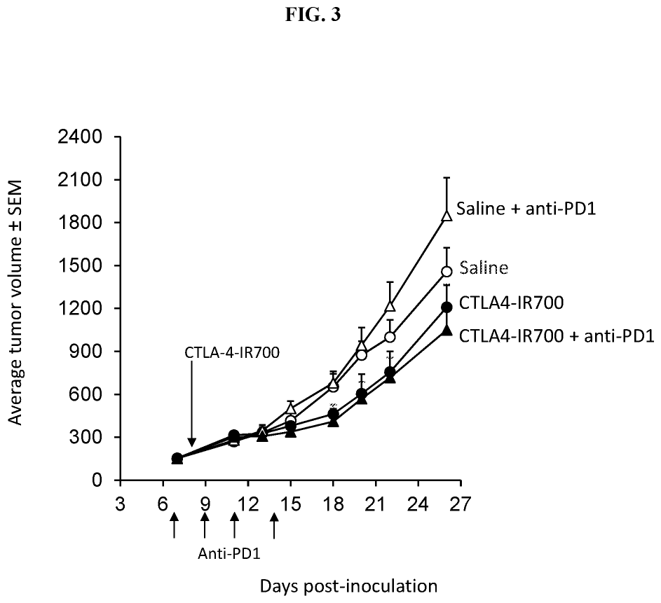

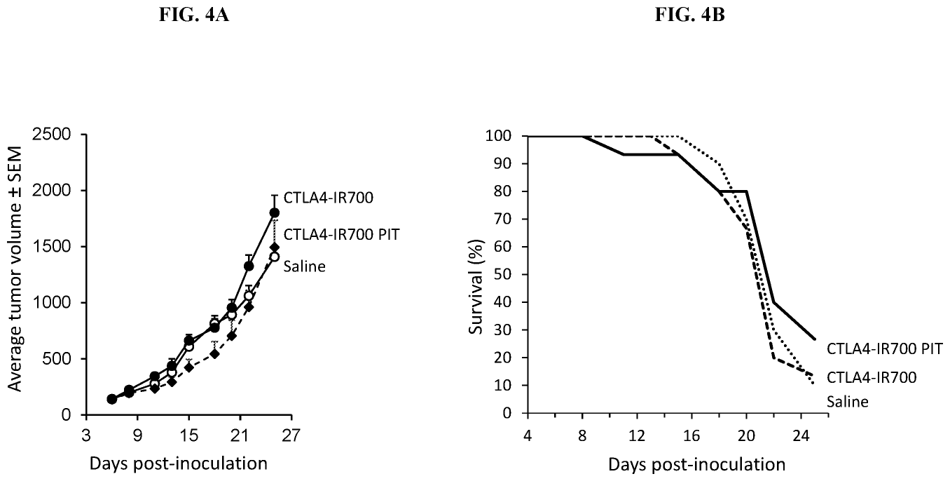

In comparison to the control group (saline), neither the anti-CTLA-4-IR700 conjugate (without PIT) nor anti-CTLA-4 PIT substantially reduced the growth of tumors (FIG. 4A). The survival of animals treated with anti-CTLA-4 PIT, however, was slightly increased compared to the conjugate alone or control (saline) (FIG. 4B).

### Example 6: Anti-CTLA-4 PIT Sensitizes Cold Tumors to Anti-PD-1 Antibody Treatment

This example describes that anti-CTLA-4 PIT can sensitize “cold” tumors, i.e. tumors with diminished immunoresponsiveness, to immune checkpoint inhibitor anti-PD-1 treatment.

BALB/c mice at age of 6-8 weeks were inoculated with 1×10⁶ 4 T1 cells per mouse subcutaneously on the right hind flank. When allograft tumors grew to a volume of about 150 mm³ (6 days after tumor cell inoculation), mice were administered saline (100 μL), anti-CTLA-4 (9H10)-IR700 conjugate (100 μg), or a combination of anti-CTLA-4 (9H10)-IR700 conjugate and anti-PD-1 antibody RMP1-14 (100 μg each). The anti-CTLA-4-IR700 conjugate was administered at Day 6 and RMP1-14 was administered at Days 6, 8, 10, and 13. Twenty-four hours after administration of the conjugate, tumors in the anti-CTLA-4 PIT group were illuminated at 690 nm at a dosage of 100 J/cm². The tumor growth was observed over 20 days, and the tumor volume was calculated using a formula: tumor volume=(width×length)×height/2.

Anti-CTLA-4 PIT in combination with anti-PD-1 substantially inhibited the growth of the “cold” tumors, though anti-CTLA-4 PIT alone did not show a substantial inhibitory effect on the growth of the “cold” tumors (FIG. 5). These data support the finding that anti-CTLA-4 PIT sensitizes cold tumors to anti-PD-1 treatment.

### Example 7: Anti-CTLA-4 PIT Sensitizes Distal Cold Tumors to Anti-PD-1 Antibody Treatment

This example describes the inhibitory effect of anti-CTLA-4 PIT in combination with anti-PD-1 antibody on the growth of distal tumors that are not directly illuminated.

BALB/c mice at age of 6-8 weeks were inoculated with 1×10⁶ 4 T1 cells per mouse subcutaneously on both the right and left hind flanks. When allograft tumors on both sides grew to volumes of about 150 mm³ (6 days after tumor cell inoculation), mice were administered saline (100 μL), anti-CTLA-4 (9H10)-IR700 conjugate (100 μg), or a combination of anti-CTLA-4-IR700 conjugate and anti-PD-1 antibody RMP1-14 (100 μg each). The anti-CTLA-4-IR700 conjugate was administered at Day 6 and RMP1-14 was administered at Days 6, 8, 10, and 13. Twenty-four hours after administration of the conjugate, tumors in the right flank in the anti-CTLA-4 PIT group were illuminated at 690 nm at a dosage of 100 J/cm², while tumors in the left flank were shielded from illumination. The tumor growth of the non-illuminated, distal tumor was observed over 20 days, and tumor volume was calculated using the formula: tumor volume=(width×length)×height/2.

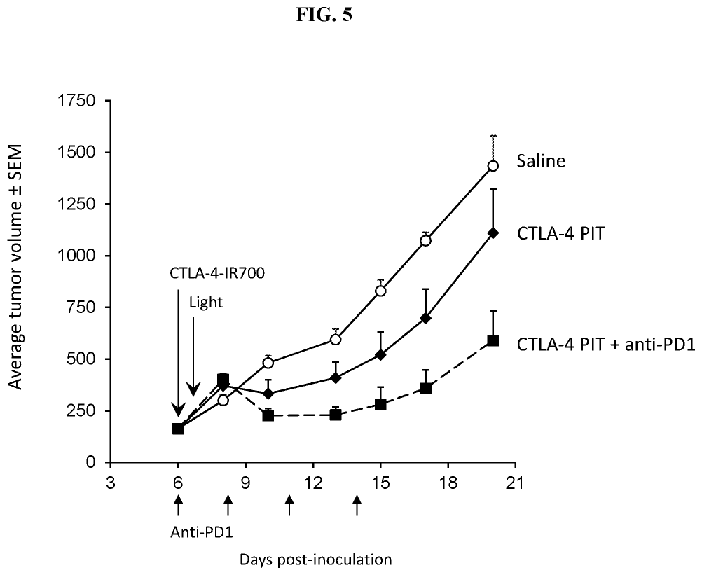

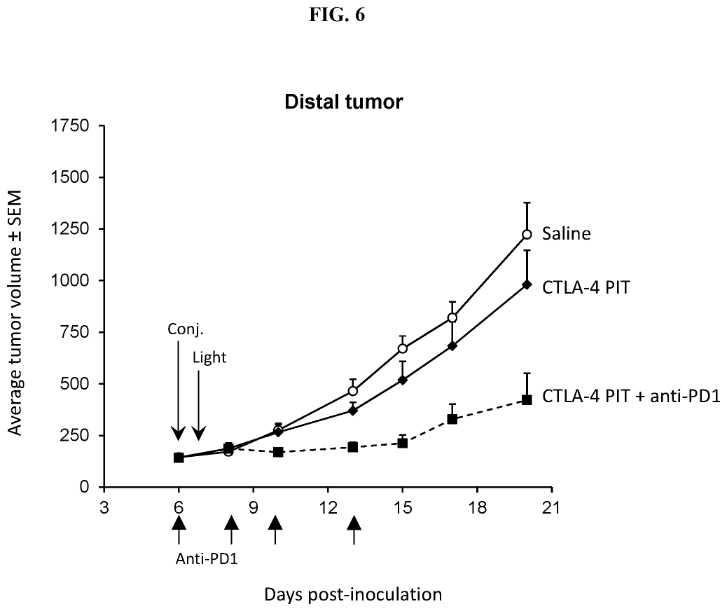

The results showed that the growth of non-illuminated, distal 4T1 tumors was substantially inhibited by the combination of anti-CTLA-4 PIT and anti-PD-1, while anti-CTLA-4 PIT alone did not inhibit distal tumor growth (FIG. 6). These data support the finding that the combination of anti-CTLA-4 PIT and anti-PD-1 immune checkpoint inhibitor is able to induce a systemic immune response and exhibit an abscopal effect, e.g., growth inhibition of a distal tumor that was not directly illuminated.

### Example 8: The Effect of Anti-CTLA-4 on Systemic Regulatory T Cells

This example describes that administration of anti-CTLA-4-IR700 conjugate (no PIT) does not affect the population of regulatory T cells (Tregs).

The percentage of FoxP3⁺ regulatory T cells (FoxP3 Treg) among CD3⁺CD4⁺ cells was determined from the spleen of the animals treated with anti-CTLA-4 clone 9H10 or anti-CD25 clone PC61 (to serve as a positive control for systemic Treg reduction). As shown in FIG. 7, the administration of anti-CTLA-4 clone 9H10 does not reduce systemic regulatory T cells, indicating that the anticancer activity of anti-CTLA-4 PIT in target and distal tumors does not require reduction of systemic regulatory T cells.

### Example 9: Anti-CTLA-4 PIT Effect on Intratumoral Regulatory T Cells

This example describes the depletion of regulatory T cells (Tregs) in vivo in response to anti-CTLA-4-IR700 PIT.

BALB/c mice were inoculated with 1×10⁶ 4 T1-EpCAM tumor cells subcutaneously on the right hind flank. Once tumors reached an average volume of approximately 150 mm³, mice were treated with saline, anti-CTLA-4-IR700 conjugate alone (CTLA-4-IR700) or anti-CTLA-4-IR700 conjugate with illumination (anti-CTLA-4-IR700 PIT; CTLA-4 PIT). Twenty-four hours after administration of the conjugate, tumors in the mice of the illuminated (PIT) group were exposed to 690 nm light at 100 J/cm². Two hours and 7 days post illumination, tumors were excised from all groups and processed into single cell suspensions. Suspended cells were then stained the T_{reg} markers CD3, CD4, CD45, and FoxP3. The stained cells were analyzed using flow cytometry, and the percentage of CD3⁺CD4⁺FoxP3⁺ cells out of CD45⁺ cells was determined.

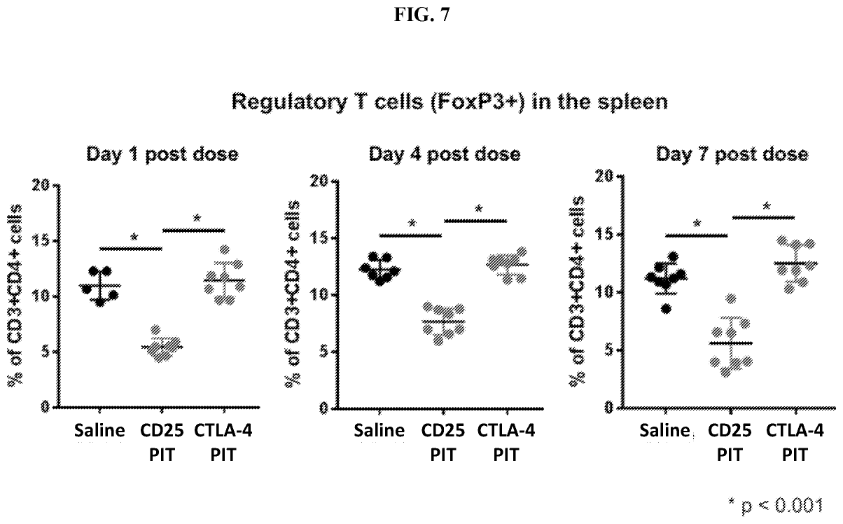

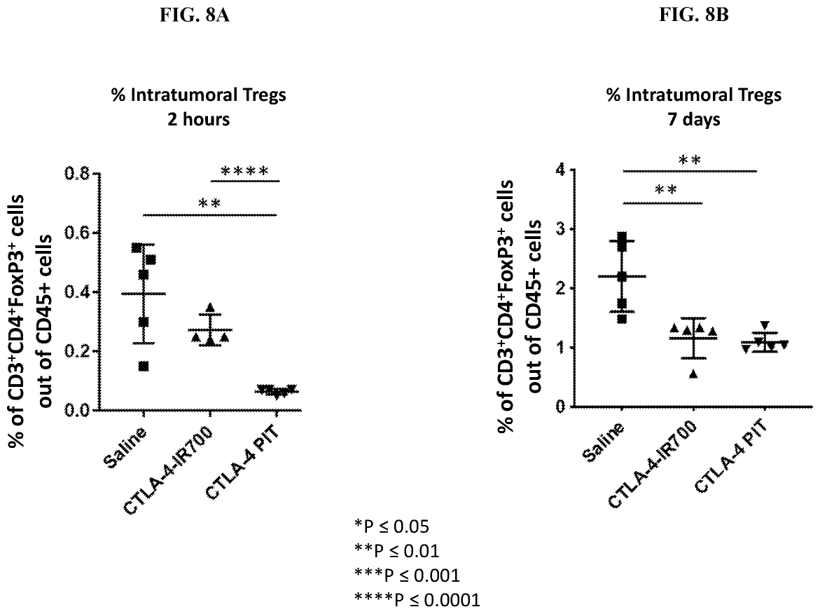

At 2 hours post treatment, tumor-bearing mice treated with anti-CTLA-4-IR700 PIT exhibited significantly decreased percentages of intratumoral CD3⁺CD4⁺FoxP3⁺ T cells in comparison to those treated with saline or anti-CTLA-4-IR700 conjugate alone (P≤0.01 and P≤0.0001, respectively), indicating an immediate Treg reduction in the tumor after anti-CTLA-4 PIT (FIG. 8A). After 7 days, tumors treated with anti-CTLA-4-IR700 PIT continued to contain a decreased percentage of intratumoral CD3⁺CD4⁺FoxP3⁺ T cells compared to tumors of control (saline-treated) animals (FIG. 8B; P<0.01). Tumors from animals treated with conjugate alone also contained a decreased percentage of intratumoral CD3⁺CD4⁺FoxP3⁺ T cells compared to tumors of control (saline-treated) animals after 7 days (P≤0.01) and similar percentages as CTLA-4 PIT treated tumors (FIG. 8B). These results demonstrated that an anti-CTLA-4 PIT leads to rapid and sustained depletion of intratumoral regulatory T cells (T_{regs}).

### Example 10: CTLA4 PIT Effect on Intratumoral CD8:Treg Ratio

This example describes the effect of the anti-CTLA-4-IR700 PIT, on the ratio of intratumoral CD8⁺ T cells to regulatory T cells (T_{regs}) in vivo, which is a predictive marker of clinical response to treatment.

BALB/c mice were inoculated with 1×10⁶ 4 T1-EpCAM tumor cells subcutaneously on the right hind flank. Once tumors reached an approximate average volume of 150 mm³, mice were treated with saline, anti-CTLA-4 (9H10)-IR700 conjugate (100 μg) alone (CTLA-4-IR700), or anti-CTLA-4-IR700 conjugate (100 μg) with illumination (anti-CTLA-4-IR700 PIT; CTLA-4 PIT). Twenty-four hours after administration of the conjugate, tumors in the mice of the illumination (PIT) group were illuminated at 690 nm at 100 J/cm². At two hours or seven days post illumination, tumors were excised from all groups and processed into single cell suspensions. Suspended cells were then stained for cell markers including CD3, CD45, CD8, CD4, and FoxP3. Isotype controls were also used for staining. The stained cells were analyzed using flow cytometry, and the ratio of intratumoral CD8⁺ T cells to T_{regs} was determined.

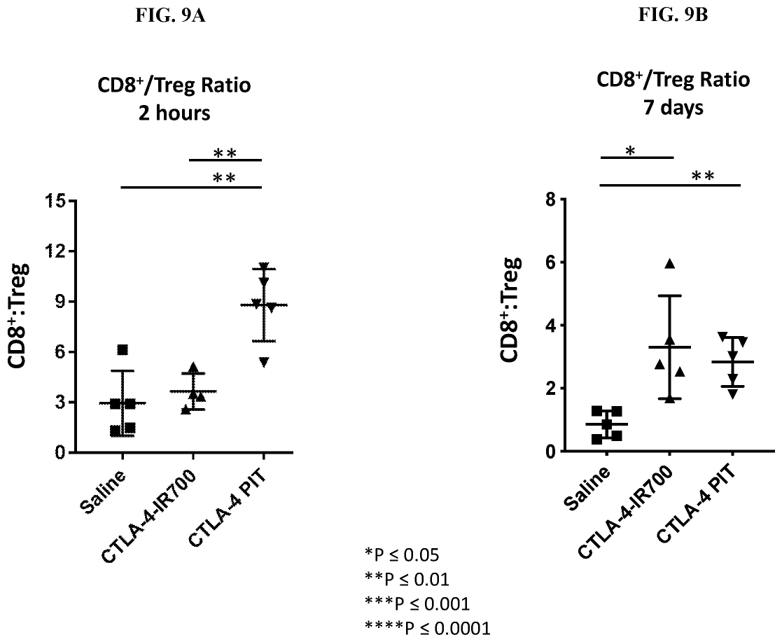

As shown in FIG. 9A, at 2 hours post illumination, tumors of mice treated with anti-CTLA-4-IR700 PIT had an increased intratumoral CD8⁺:T_{reg} ratio compared to mice that received saline (P≤0.01) or anti-CTLA-4-IR700 conjugate alone (without illumination) (P≤0.01). The increased CD8⁺:T_{reg} ratio in tumors of animal receiving PIT was sustained through seven days post illumination (FIG. 9B; P≤0.01). Administration of anti-CTLA-4 conjugate alone resulted in an increased CD8⁺:T_{reg} ratio, compared to mice that received saline by 7 days post illumination (FIG. 9B; P≤0.05). These results indicate a single treatment of anti-CTLA-4 PIT results in a rapid and durable increase in CD8⁺:T_{reg} ratio inside the treated tumor.

### Example 11: Anti-CTLA-4-IR700 PIT Results in Rapid Increase in Activated CD8⁺ T Cells

This example describes the effect of anti-CTLA-4-IR700 PIT on intratumoral CD8⁺ T cell activation in vivo.

BALB/c mice were inoculated with 4T1-EpCAM tumor cells. Once tumors reached an approximate average volume of 150 mm³, mice were treated with saline, anti-CTLA-4 (9H10)-IR700 conjugate (CTLA-4-IR700) alone (100 μg), or anti-CTLA-4-IR700 conjugate (100 μg) with illumination (anti-CTLA-4-IR700 PIT; CTLA-4 PIT). Twenty-four hours after administration of the conjugate, tumors in the mice of the illumination (PIT) group were illuminated at 690 nm at 100 J/cm². Two hours post illumination, tumors were excised from all groups and processed into single cell suspensions. Suspended cells were then stained for cell markers for cell type identification and analyzed by flow cytometry. The percent CD25⁺ cells out of CD8 T cells was determined for each condition.

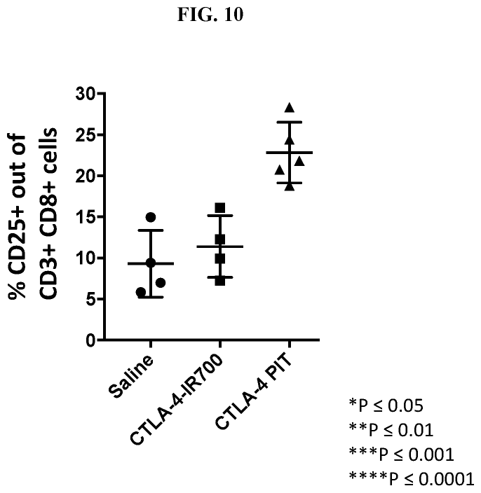

As shown in FIG. 10, in mice treated with anti-CTLA-4-IR700 PIT (triangles), the number of activated CD8⁺ T cells (CD3⁺CD8⁺CD25⁺) was significantly increased (P≤0.01) compared to mice that received saline (circles) or anti-CTLA-4-IR700 conjugate alone (squares). These results indicate that anti-CTLA-4 PIT results in rapid increased activated CD8⁺ T cells in the illuminated tumor.

### Example 12: Anti-CTLA-4-IR700 PIT Results in Sustained Increased Activated CD8⁺ T Cells

This example describes the effect of anti-CTLA-4-IR700 PIT on sustained intratumoral CD8⁺ T cell activation in vivo.

BALB/c mice were inoculated with 4T1-EpCAM tumor cells. Once tumors reached an approximate average volume of 150 mm³, mice were treated with saline, anti-CTLA-4 (9H10)-IR700 conjugate (CTLA-4-IR700) alone (100 μg), or anti-CTLA-4-IR700 conjugate (100 μg) with illumination (anti-CTLA-4-IR700 PIT; CTLA-4 PIT). Twenty-four hours after administration of the conjugate, tumors in the mice of the illumination (PIT) group were illuminated at 690 nm at 100 J/cm². Seven days post illumination, tumors were excised from all groups and processed into single cell suspensions. Suspended cells were then stained for cell markers including CD3, CD69, CD45, CD8, and Ki67. Isotype controls were also used for staining. The stained cells were analyzed using flow cytometry.

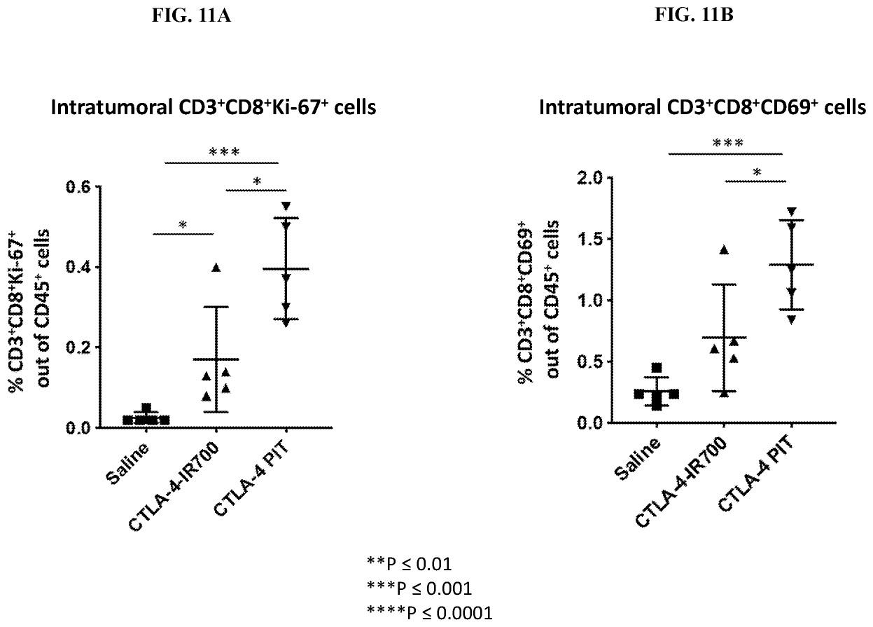

As shown in FIGS. 11A and 11B in mice treated with anti-CTLA-4-IR700 PIT (inverse triangles), the percentage of activated CD8⁺ T cells (CD3⁺CD8⁺Ki67⁺, FIG. 11A; and CD3⁺CD8⁺CD69⁺, FIG. 11B) was significantly increased (P<0.001) compared to mice that received saline (squares) or anti-CTLA-4-IR700 conjugate alone (triangles). These results indicate that anti-CTLA-4 PIT results in sustained increased activated CD8⁺ T cells in the illuminated tumor.

### Example 13: Anti-CTLA-4-IR700 PIT Results in Increased Activated Natural Killer Cells

This example describes the effect of anti-CTLA-4-IR700 PIT on sustained intratumoral natural killer (NK) cell activation in vivo.

BALB/c mice were inoculated with 4T1-EpCAM tumor cells. Once tumors reached an approximate average volume of 150 mm³, mice were treated with saline, anti-CTLA-4 (9H10)-IR700 conjugate alone (100 μg), or anti-CTLA-4-IR700 conjugate (100 μg) with illumination (anti-CTLA-4-IR700 PIT). Twenty-four hours after administration of the conjugate, tumors in the mice of the illumination (PIT) group were illuminated at 690 nm at 100 J/cm². Seven days post illumination, tumors were excised from all groups and processed into single cell suspensions. Suspended cells were then stained for cell markers including CD3, CD69, CD45, CD49b, and Ki67. Isotype controls were also used for staining. The stained cells were analyzed using flow cytometry.

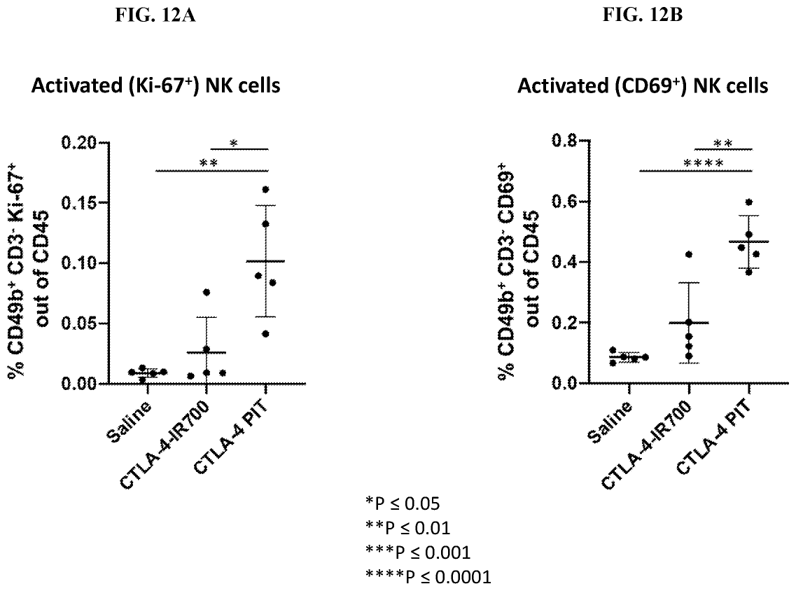

As shown in FIGS. 12A and 12B in mice treated with anti-CTLA-4-IR700 PIT, the proportion of activated NK cells (CD49b⁺CD3⁻Ki67⁺, FIG. 12A; and CD49b⁺CD3⁻CD69⁺, FIG. 12B), shown as a percentage of CD45⁺ cells, was significantly increased compared to mice that received saline (P≤0.01 and P≤0.0001, respectively) or anti-CTLA-4-IR700 conjugate alone (P≤0.05 and P≤0.01, respectively). These results demonstrate that anti-CTLA-4 PIT results in increased activated NK cells in the illuminated tumor.

### Example 14: Anti-CTLA-4-IR700 PIT Enhances Expansion of Tumor Antigen-Specific Cytotoxic Lymphocytes in the Periphery

This example describes the stimulatory effect of anti-CTLA-4-IR700 PIT on systemic immunity in vivo.

A. Cytotoxic T Lymphocyte (CTL) Assay

CTL assay was designed to evaluate the tumor-specific cytotoxic activity of splenocytes from mice inoculated with CT26-EphA2 clone c4D10 tumors. Cytotoxicity was evaluated using the CytoTox™ 96 Non-Radioactive Cytotoxicity Assay (Promega; Cat. #G1780). The kit measured the levels of lactate dehydrogenase (LDH) in the well, which is released from cells upon cell death. The spleens were harvested from the tumor-bearing mice that were treated with anti-CTLA-4 (9H10)-IR700 conjugate alone (100 μg), or anti-CTLA-4-IR700 (CTLA-4 IR700) conjugate (100 μg) with illumination (anti-CTLA-4-IR700 PIT; CTLA-4 PIT), or from control tumor-bearing mice administered saline. Single-cell suspensions were prepared by mechanical dissociation of harvested spleens over a 70-μm pore size cell strainer. The resulting flow-through was collected, and red blood cells were lysed. The suspended splenocytes were primed with the CT26 antigen AH1 peptide for four days in vitro. Afterwards, a cytotoxic assay was performed by co-incubating the splenocytes (effector cells) and CT26-ephA2 clone c4D10 target cells at several effector cell to target cell ratios (E:T ratios) for four hours. Subsequently, the splenocytes were removed, and the LDH levels released from the target cells were measured. The human pancreatic cancer cell line BxPC3 cells were used as unrelated control target cells.

B. Results

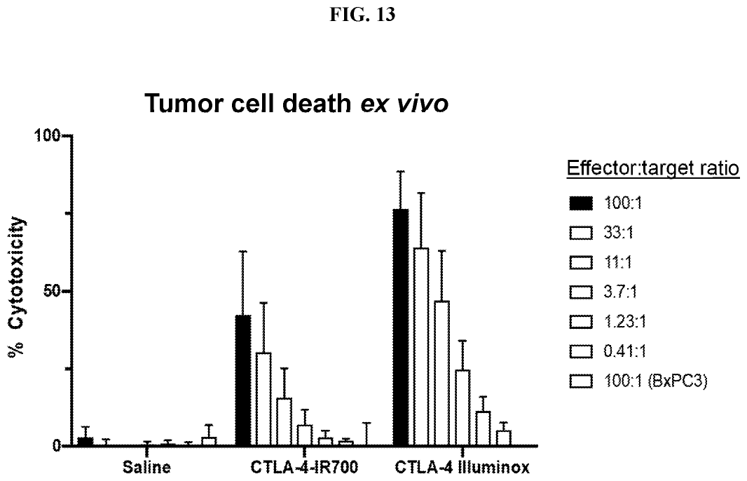

In mice that were treated with conjugate alone or anti-CTLA-4-IR700 PIT, the splenocytes exhibited tumor-specific immune response against the target tumor cells ex vivo (FIG. 13). For splenocytes derived from mice that were treated with anti-CTLA-4-IR700 PIT, the results showed a clear E:T ratio-dependent cytotoxic effect on the target tumor cells, capable of killing more than 75% of the target cells at an E:T ratio of 100:1, and about 60% at a ratio of 33:1 (FIG. 13). For splenocytes derived from mice treated with anti-CTLA-4-IR700 conjugate alone (CTLA-4-IR700; without PIT), the results showed a clear E:T ratio-dependent cytotoxic effect on the target tumor cells, capable of killing about 40% of the target cells at a ratio of 100:1, and about 30% at a ratio of 33:1. For splenocytes derived from control, saline-treated, mice, the results showed a minimal cytotoxic effect on the target tumor cells at any E:T ratio (FIG. 13). Moreover, there was essentially no cytotoxic effect against BxPC3 cells, an unrelated type of tumor cells serving as a control for target tumor cells at an E:T ratio of 100:1. These results clearly showed that treatment with anti-CTLA-4-IR700 PIT resulted in an increase in tumor-specific cytotoxic T cell activity in the spleen, and the increase in systemic immunoactivity was substantially greater than the increase in tumor-specific cytotoxic T cell activity in mice treated with anti-CTLA-4-IR700 conjugate without light treatment. These results indicate that the light-activation of the conjugate within the tumor contributes additional systemic immunoactivity over the functionality provided by solely the anti-CTLA4 antibody component.

### Example 15: Rejection of the Growth of Re-Challenged Tumors in Mice with Complete Response after Anti-CTLA-4-IR700 PIT

This example describes the rejection of growth of tumors newly inoculated into the mice that had achieved complete response after initial treatment with an exemplary anti-CTLA-4-IR700 PIT.

BALB/c mice at age of 6-8 weeks were inoculated with 1×10⁶ CT26-EphA2 clone c4D10 cells/mouse subcutaneously on the right and left hind flank simultaneously. When allograft tumors grew to a size of about 250 mm³, mice were administered anti-CTLA-4-IR700 conjugate (100 μg). Twenty-four hours after administration of anti-CTLA-4-IR700, tumors were illuminated at 690 nm at 100 J/cm². The mice that achieved complete response (with disappearance of tumors from both right and left hind flanks) (CR mice; n=7) were re-challenged, and naïve mice (n=10) were inoculated, with CT26-EphA2 cells on the left hind flank 72 days after the initial tumor cell inoculation (i.e., 63 days post anti-CTLA-4 PIT treatment). The growth of the tumors from the newly inoculated cells were observed for up to 44 days, and tumor volumes were calculated using the formula: tumor volume=(width×length)×height/2.

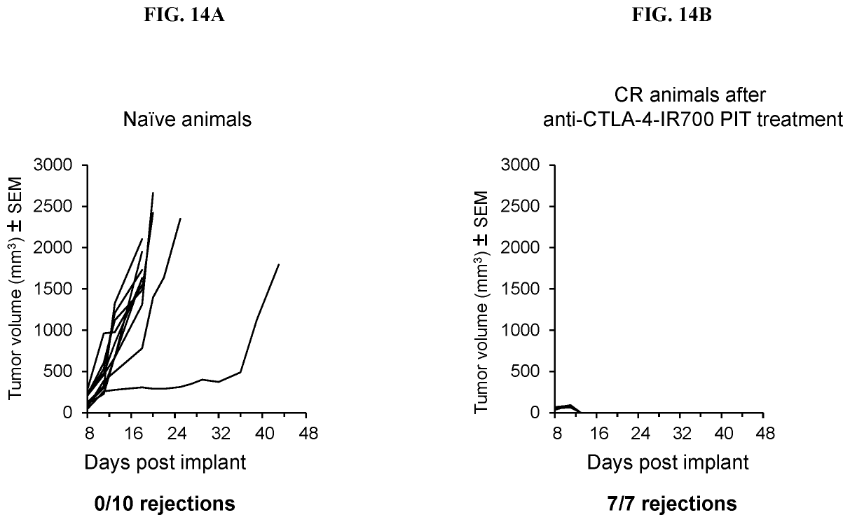

All naïve mice (10/10), not previously exposed to any treatment, developed tumors that exhibited continuous growth (FIG. 14A). In contrast, all anti-CTLA-4 PIT-treated animals (7/7) completely rejected the second inoculation of tumor cells, and no tumor growth was observed beyond 10 days post-inoculation, and any initial growth observed returned to baseline by about 13 days (FIG. 14B). These results indicate anti-CTLA-4 PIT treatment enhances systemic antitumor immunity in animals.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of treating a tumor or a lesion that is non-responsive to or resistant to a prior immune checkpoint inhibitor therapy, the method comprising:
(a) identifying a tumor or a lesion in a subject that is non-responsive to or resistant to treatment with a prior immune checkpoint inhibitor;
(b) administering to the subject a conjugate comprising a phthalocyanine dye linked to a targeting molecule that binds to CTLA-4;
(c) after administering the conjugate, illuminating the tumor or the lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and
(d) administering a first immune checkpoint inhibitor,
wherein the tumor or the lesion exhibits sensitivity to the first immune checkpoint inhibitor.

2. The method of claim 1, wherein sensitivity to the first immune checkpoint inhibitor comprises a reduction in volume, dimensions or mass of the tumor or the lesion, a less than 20% increase in volume or dimensions of the tumor or the lesion, or a reduction in the number of tumor cells.

3. The method of claim 1, wherein sensitivity to the first immune checkpoint inhibitor comprises a reduction in tumor cell metastasis, an increase in tumor cell killing, an increase in systemic immune response, an increase in new T cell priming, an increase in diversity of CD8⁺ T cells or any combinations thereof.

4. The method of claim 3, wherein sensitivity to the first immune checkpoint inhibitor comprises an increase in systemic immune response, and the systemic immune response is measured by one or more of a cytotoxic T lymphocyte (CTL) activity assay, an intratumoral T cell exhaustion assay, an intratumoral effector T cell expansion assay, a T cell receptor diversity assay, an activated CD8⁺ T cell assay, a circulating regulatory T cell (Treg) assay, an intratumoral Treg assay, or a CD8⁺ Tcell:Treg assay.

5. The method of any of claims 1-4, wherein the tumor or the lesion that is non-responsive or resistant is identified by a high mutational burden or a tumor immune score.

6. The method of any of claims 1-4, wherein the tumor or the lesion that is non-responsive or resistant is identified by status of expression of a PD-1 or a PD-L1 biomarker.

7. The method of any of claims 1-6, wherein the tumor or the lesion that is non-responsive or resistant is identified by a liquid biopsy or a tissue biopsy.

8. The method of any of claims 1-7, wherein the treatment with the prior immune checkpoint inhibitor comprises treatment with a PD-1 inhibitor, a PD-L1 inhibitor or a CTLA-4 inhibitor.

9. The method of any of claims 1-8, wherein the treatment with the prior immune checkpoint inhibitor comprises treatment with an anti-PD-1 antibody or antigen-binding fragment thereof.

10. The method of claim 9, wherein the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

11. A method of provoking a systemic immune response, the method comprising:
(a) administering to a subject a conjugate comprising a phthalocyanine dye linked to a targeting molecule that binds to CTLA-4;
(b) after administering the conjugate, illuminating at the site of a first tumor or a first lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and
(c) administering a first immune checkpoint inhibitor,
wherein following steps (a), (b), and (c), the subject exhibits at least one systemic immune responsive feature in a location distal to the illuminated site.

12. The method of claim 11, wherein the at least one systemic immune responsive feature is selected from the group consisting of an increase in CD8⁺ T cell infiltration, an increase in CD8⁺ T cell activation, an increase in the CD8⁺:Treg ratio, an increase in natural killer cell infiltration, an increase in natural killer cell activation, an increase in dendritic cell infiltration, an increase in dendritic cell activation, an increase in new T cell priming, an increase in T cell diversity, and any combination thereof.

13. The method of claim 11, wherein the at least one systemic immune responsive feature comprises an increase in one or more of a proinflammatory molecule, a proinflammatory cytokine, or an immune cell activation marker.

14. The method of any of claims 11-13, wherein the at least one systemic immune responsive feature is assessed from a blood sample obtained from the subject.

15. The method of any of claims 11-14, wherein the location distal to the illuminated site is a second tumor or a second lesion that is not illuminated.

16. A method of provoking a local immune response comprising:
(a) administering to a subject a conjugate comprising a phthalocyanine dye linked to a targeting molecule that binds to CTLA-4;
(b) after administering the conjugate, illuminating the tumor or the lesion at a wavelength of at or about 600 nm to at or about 850 nm and at a dose of from at or about 25 J/cm² to at or about 400 J/cm² or from at or about 2 J/cm fiber length to at or about 500 J/cm fiber length; and
(c) administering a first immune checkpoint inhibitor,
wherein following steps (a), (b), and (c), the subject exhibits at least one local immune responsive feature, and wherein the at least one local immune responsive feature is synergistic as compared to administering only the first immune checkpoint inhibitor or as compared to treatment only with the conjugate and the illuminating step.

17. The method of claim 16, wherein the at least one local immune responsive feature is selected from the group consisting of intratumoral Treg depletion, an increase in intratumoral CD8 T cell infiltration, an increase in intratumoral CD8 T cell activation, an increase in the intratumoral CD8⁺:Treg ratio, an increase in intratumoral natural killer cell infiltration, an increase in intratumoral natural killer cell activation, a decrease in myeloid suppressive cells, a Type I interferon response, and any combination thereof.

18. The method of claim 16, wherein the at least one local immune responsive feature comprises an increase in an anti-immune cell type or an immune activation marker in the tumor or tumor microenvironment.

19. The method of any of claims 1-18, wherein the targeting molecule comprises an anti-CTLA-4 antibody or an antigen binding fragment thereof.

20. The method of claim 19, wherein the anti-CTLA-4 antibody is selected from the group consisting of ipilimumab (YERVOY), tremelimumab, AGEN1181, AGEN1884, ADU-1064, BCD-145, CBT-509, and BCD-217.

21. The method of any of claims 1-20, wherein the first immune checkpoint inhibitor comprises an anti-PD-1 antibody or antigen-binding fragment thereof.

22. The method of claim 21, wherein the first immune checkpoint inhibitor is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011), and antigen-binding fragments thereof.

23. The method of any of claims 1-22, wherein the first immune checkpoint inhibitor is administered concurrently with the administering the conjugate.

24. The method of any of claims 1-22, wherein the first immune checkpoint inhibitor is administered within 24 hours of administering the conjugate.

25. The method of any of claims 1-22, wherein the first immune checkpoint inhibitor is administered prior to administering the conjugate.

26. The method of claim 25, wherein the first immune checkpoint inhibitor is administered between about 1-3 weeks prior to administering the conjugate.

27. The method of claim 25 or claim 26, wherein the first immune checkpoint inhibitor is administered 1, 2, 3, 4, 5 times, or more than 5 times prior to administering the conjugate.

28. The method of any of claims 1-27, further comprising administering the first immune checkpoint inhibitor subsequent to administering the conjugate.

29. The method of claim 28, wherein the first immune checkpoint inhibitor is administered 1, 2, 3, 4, 5 times, or more than 5 times subsequent to administering the conjugate.

30. The method of claim 28 or claim 29, wherein the first immune checkpoint inhibitor is administered between about 1 day and about 4 weeks after administering the conjugate.

31. The method of any of claims 1-10 and 19-30, wherein the subject exhibits progressive disease or a stable disease following treatment with a prior immune checkpoint inhibitor.

32. The method of any of claims 1-10 and 19-30, wherein the tumor or the lesion that is non-responsive to or resistant to a prior immune checkpoint inhibitor therapy comprises a tumor or a lesion that exhibits a lack of reduction in volume, dimensions or mass of the tumor or the lesion, more than 20% increase in volume or dimensions of the tumor or the lesion, or an increase in the number of tumor cells, or a metastases.

33. The method of any of claims 1-32, wherein the subject comprises a second tumor or lesion that is not illuminated, and wherein the second tumor or lesion exhibits sensitivity to administering the first immune checkpoint inhibitor.

34. The method of any of claims 1-32, wherein the subject comprises metastatic tumor cells and wherein the metastatic tumor cells exhibit sensitivity to administering the first immune checkpoint inhibitor.

35. The method of any of claims 1-34, wherein the subject does not experience a substantial reduction in systemic Treg cells.

36. The method of any of claims 1-35, wherein the subject exhibits a response at a site distal to the illuminated tumor or lesion, wherein the response is selected from the group consisting of an increase in CD8⁺ T cell infiltration, an increase in CD8⁺ T cell activation, an increase in the intratumoral CD8⁺:Treg ratio, an increase in intratumoral natural killer cell infiltration, an increase in intratumoral natural killer cell activation, an increase in dendritic cell infiltration, an increase in dendritic cell activation, an increase in new T cell priming, an increase in T cell diversity, increase in one or more of a proinflammatory molecule, a proinflammatory cytokine, an immune cell activation marker, and any combination thereof.

37. The method of any of claims 1-36, wherein the method results in a substantial decrease in the number, the frequency, the activity and/or the function of an intratumoral suppressor cell.

38. The method of claim 37, wherein the intratumoral suppressor cell is selected from the group consisting of regulatory T cells, type II natural killer T cells, M2 macrophages, tumor associated fibroblast, myeloid-derived suppressor cell, and any combination thereof.

39. The method of any of claims 1-38, wherein the method results in a substantial increase in the number or the frequency of intratumoral cytotoxic T effector cells, natural killer (NK) cells, other immune effector cells, or any combination thereof.

40. The method of any of claims 1-39, wherein the method results in in a substantial increase in the activity or the function of intratumoral cytotoxic T effector cells, natural killer (NK) cells, other immune effector cells, or any combination thereof.

41. The method of any of claims 1-40, wherein necrosis of the tumor or the lesion occurs following the illuminating step.

42. The method of any of claims 1-41, wherein the phthalocyanine dye is a Si-phthalocyanine dye.

43. The method of claim 42, wherein the Si-phthalocyanine dye is IR700.

44. The method of any of claims 1-43, wherein the illuminating step is carried out between 30 minutes and 96 hours after administering the conjugate.

45. The method of any of claims 1-44, wherein the illuminating step is carried out 24 hours±4 hours after administering the conjugate.

46. The method of any of claims 1-45, wherein the illuminating step is carried out at a wavelength of 690±40 nm.

47. The method of any of claims 1-46, wherein the illuminating step is carried out at a dose of or about of 50 J/cm² or 100 J/cm of fiber length.

48. The method of any of claims 1-47, wherein the administration of the conjugate is repeated one or more times, optionally wherein after each repeated administration of the conjugate, the illuminating step is repeated.

49. The method of any of claims 1-48, further comprising administering an additional therapeutic agent or anti-cancer treatment.

50. The method of any of claims 1-49, wherein the tumor or the lesion is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.
